(19) United States
(12) Patent Application Publication
(10) Pub. No.: US 2022/0402914 A1
(43) Pub. Date: Dec. 22, 2022

(54) Tryptoline-Based Benzothiazoles and their use as Antibiotics and Antibiotic Resistance-Modifying Agents

(71) Applicant: The Regents of the University of Colorado, a body corporate, Denver, CO (US)

(72) Inventors: Xiang Wang, Superior, CO (US); Xinfeng Wang, Boulder, CO (US)

(21) Appl. No.: 17/639,016

(22) PCT Filed: Sep. 3, 2020
(86) PCT No.: PCT/US2020/049163

§ 371 Date: Feb. 28, 2022

Related U.S. Application Data
(60) Provisional application No. 62/895,380, filed on Sep. 3, 2019.

(51) Int. Cl. C07D 471/04

(52) U.S. Cl. CPC C07D 471/04

(57) ABSTRACT

The present inventions relates to tryptoline-based benzothiazole compounds and their use as both novel resistance modifying agents, and antibiotics.

This International PCT Application claims the benefit of and priority to U.S. Provisional Application No. 62/895,380, filed Sep. 3, 2019. The entire specification and figures of the above-referenced application is hereby incorporated, in its entirety by reference.

### STATEMENT OF FEDERALLY SPONSORED RESEARCH

This invention was made with government support under grant number AI121581 awarded by the National Institutes of Health. The government has certain rights in the invention.

### TECHNICAL FIELD

The present inventions relates to tryptoline-based benzothiazole compounds and their use as both novel resistance modifying agents, and antibiotics. In particular, the tryptoline-based benzothiazole compounds of the invention have antibacterial activity and/or are capable of re-sensitizing methicillin-resistant S. aureus to a variety of p-lactam antibiotics. The present invention also relates to a method for producing and using the same.

### BACKGROUND

Bacterial antibiotic resistance is a world-wide health concern. Among the growing incidence of antibiotic resistant infectious bacteria, methicillin-resistant Staphylococcus aureus (MRSA) is the most frequently identified resistant pathogen in US hospitals and is associated with exceptionally high rates of morbidity and mortality due to hospital acquired infections (HAIs). In fact, a recent report released by the Centers for diseases control and prevention (CDC) estimates that nearly 119,000 noninvasive MRSA infections directly caused more than 20,000 deaths in the 2017 alone. Furthermore, MRSA infections account for more deaths in the United States than HIV/AIDS and tuberculosis combined. These issues illustrate the necessity for continuous discovery of new antibiotics classes with novel structures and mechanisms of action. Although efforts have been in place to further new antibiotic discovery, only a few antibiotics with novel modes of action have been brought to the clinic in the last 50 years. This is largely because the rate of antibiotic resistance development has surpassed the rate at which new antibiotics are being discovered and developed. Resistance-modifying agents (RMAs) offer a promising alterative strategy to combat bacterial resistance. An RMA is a treatment alternative which does not kill or inhibit the growth of bacteria on its own but enhances the antibiotic activity of already established antibacterial drugs. A notable advantage of the RMAs is that they can extend the market lifespan of known antibiotics that have already been optimized for large-scale production with well-studied toxicity profiles.

Previously, the present inventors demonstrated that compounds containing the tricyclic indoline and chlorobenzene fragment are capable of acting as RMAs in combination with p-lactam antibiotics against MRSA (Of1, FIG. 1). The present inventors showed that structures with bridged tetracyclic indolenine also sensitize a variety of MRSA strains to β-lactams (2, FIG. 1). Furthermore, an aza-tricyclic indoline (3, FIG. 1) was developed to optimize the physiochemical properties of Of1 while maintaining the RMA activity. Since 3 still possesses the RMA properties of 1 while providing a site for facile modification, the present inventors used this compound as a core scaffold compound to test the effects of functionalization with different chemical moieties on anti-MRSA activities and mammalian cell toxicity.

Benzimidazole, benzoxazole, and benzothiazoles are bioactive heterocyclic compounds found in many natural products and pharmaceutical agents. These moieties represent ideal sources of core scaffolds and capping fragments for the design and synthesis of targeted molecules. As described below, the present inventors examine whether functionalization of a tricyclic-indoline core with any of the aforementioned motifs might enhance RMA activity or decrease observed cytotoxicity. Again, as detailed below, the present inventors report the discovery of tryptoline-based benzothiazoles (4, FIG. 1) as novel RMAs and antibiotics though a rigorous structure-activity relationship (SAR) studies.

### SUMMARY OF THE INVENTION

One aspects of the invention provide a resistance-modifying agent (“RMA”). As noted above, RMAs may target non-essential, resistance-conferring genes and restore antibiotic sensitivity of a bacteria. A notable advantage of RMAs is that they are capable of extending the market lifespan of known antibiotics that have already been optimized for large-scale production with well-studied toxicity profiles.

On aspect of the present invention includes tryptoline-based benzothiazoles as a novel class of RMAs, that may, in one preferred embodiment, selectively re-sensitizes methicillin-resistant S. aureus to β-lactam antibiotics, such as oxacillin, amoxicillin/clavulanic acid, meropenem and cefazolin. Tryptoline-based benzothiazole compounds of the invention can be used in combination with antibiotics, such as β-lactam antibiotics to treat antibiotic resistant bacterial infections such as MRSA. Moreover, some of the Tryptoline-based benzothiazole compounds of the invention are effective antibiotics in and of themselves.

In one preferred aspect, a variety of tryptoline-based benzothiazoles may be synthesized and used to potentiate antibiotic compounds, such as representative β-lactam antibiotics directed to MRSA. In another preferred embodiment, compound 4ad demonstrated strong RMA activity and low mammalian cytotoxicity (MRC=2 μg/mL, MIC >32 μg/mL, with GI50 >100 μg/mL). In yet another preferred embodiment, the present inventors also identified compound 12g as a novel anti-MRSA antibiotic (MRC=1 μg/mL, MIC=2 μg/mL, with GI50 of 14.1 μg/mL).

The present invention includes a new class of RMAs with a novel tryptoline-based benzothiazole scaffold. In one example, a tryptoline-based benzothiazole in this series (4ad) re-sensitizes multiple MRSA strains to cephalosporins at low concentrations (2 μg/mL) and has low mammalian cytotoxicity with a half growth inhibitory concentration (GI₅₀) >100 μg/mL in human cervical carcinoma (HeLa) cells. In addition, the present invention includes a tryptoline-based benzothiazole core scaffold which may further include various different substitutions that provide antibacterial activity against MRSA.

In one particular embodiment, the invention may include a tryptoline-based benzothiazole compound. In one particular embodiment, the tryptoline-based benzothiazole compound of the invention may be an RMA. In another particular embodiment, the tryptoline-based benzothiazole compound of the invention may be an antibiotic.

Another aspect of the invention provides a method for treating bacterial infections, such as a MRSA infection in a subject comprising administering to the subject having a. infection a therapeutically effective amount of an antibiotic, such as a β-lactam and a tryptoline-based benzothiazole compound described herein. In some embodiments, the β-lactam comprises amoxicillin, clavulanic acid, cefazolin, meropenem, or a combination thereof. Some preferred aspects of the invention provide a tryptoline-based benzothiazole compound that is capable of re-sensitizing the susceptibility of methicillin-resistant S. aureus to a β-lactam antibiotic.

Another aspect of the invention provides a method for treating bacterial infections, such as a MRSA infection in a subject comprising administering to the subject having a. infection a therapeutically effective amount of a tryptoline-based benzothiazole compound described herein.

It should be appreciated that combinations of various groups described herein form other preferred embodiments. In this manner, a variety of compounds of Formulas I-V are embodied within the present invention.

Another aspect of the invention provides an antibiotic composition comprising one or more of a compound of the invention described herein. In some embodiments, the antibiotic composition further comprises a β-lactam antibiotic. Exemplary β-lactam antibiotics include s-lactam comprises amoxicillin, clavulanic acid, cefazolin, meropenem, and a combination thereof. Yet in other embodiments, the antibiotic composition further comprises a P-lactamase inhibitor or other resistance-modifying agent or a combination thereof.

Compounds of the invention are useful in treating bacterial infection in a subject. In some embodiments, compounds of the invention are used to treat drug resistant strain bacterial infection. Yet in other embodiments, the compound of the invention is used to treat MRSA infection.

While some of the specific substituents for Compounds of the invention are disclosed herein, it should be noted that combinations of various groups described herein form other embodiments. In this manner, a variety of compounds are embodied within the present invention.

Additional aspects of the invention may be evidenced from the specification, claims and figures provided below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. tricyclic indoline and tryptoline as core structures of RMAs.

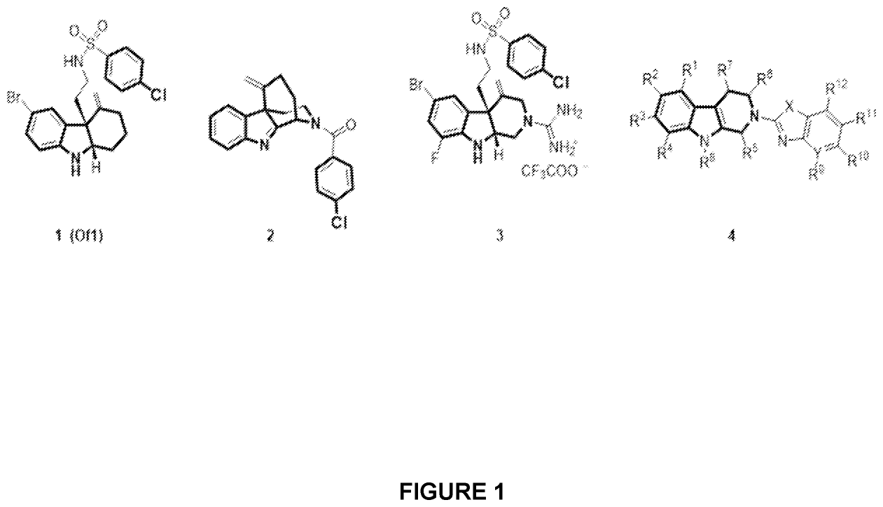

FIG. 2. SAR studies summary of tryptoline-based benzothiazoles.

FIG. 3. Scheme 1: General method for the synthesis of tryptoline-based benzothiazoles, benzimidazoles and benzoxazoles. Reagents and conditions: (a) K₂CO₃, DMF, 12 h, 90° C.; (b) DCM, 2 h, rt; (c) Pd(PPh₃)₄, MnO₂, CH₃CN, O₂, 80° C., 8 h; (d) BPO, Na₂HPO₄, DMF, 12 h, rt; (e)N-methyl benzimidazole, (TMP)ZnCl.LiCl, Cu(OAc)₂, THF, 12 h, rt.

FIG. 4. Scheme 2: Synthesis of 4aa-4ae. Reagents and conditions: (a) BBr₃, DCM, 12 h, −78° C.—rt; (b) 1)N-(2-hydroxyethyl)phthalimide, PPh₃, DIAD, THF, 15 h, reflux; 2) hydrazine hydrate, EtOH, 3 h, reflux; (c) ammonium hydroxide solution, Cu₂O, NMP, 48 h, 80° C.; (d) 1) Boc-Gly-OH, DMAP, EDCI, DCM, 2 h, rt; 2) HCl, 1,4-dioxane, 4 h, rt.

FIG. 5. Scheme 3: Synthesis of 4ah-4aj. Reagents and conditions: (a) ammonium hydroxide solution, Cu₂O, NMP, 48 h, 80° C.; (b) 1) Na, (HCHO)ₙ, MeOH, 2 h, reflux; 2) NaBH₄, MeOH, 2 h, 0° C. to reflux; (c) 1)N-Boc-2-aminoacetaldehyde, AcOH, NaHB(OAc)₃, ClCH₂CH₂Cl, 16 h, rt; 2) HCl, 1,4-dioxane, 4 h, rt.

FIG. 6. Scheme 4: Synthesis of 4l-4n, 4o and 4q. Reagents and conditions: (a) DMF, TEA, 110° C., 12 h; (b) NaH, EtI, DMF, overnight, rt; (c) Fe, AcOH, 24 h, rt.

FIG. 7. Scheme 5: ScSynthesis of 8-11 and 12a-12g. Reagents and conditions: (a) LiOH, MCOH/H₂O, 12 h, rt; (b) 1) (COCl)₂, DMF, DCM, 30 min; 2) NHMe₂, TEA, DCM, 3 h, rt; (c) LiAlH₄, THF, 12 h, 0° C. to rt; (d)MOMCl, TEA, DCM, rt; (e) 1) PPh₃, I₂, imidazole, DCM, 1 h, rt; 2) HSCH₂CH₂NHBoc, NaOH, t-BuOH, 120° C., 3 h; 3) HCl, 1,4-dioxane, rt; (f) 1) PPh₃, I₂, imidazole, DCM, 1 h, rt; 2) NH₃H₂O, t-BuOH, 120° C., 3 h; (g) 1) PPh₃, I₂, imidazole, DCM, 1 h, rt; 2) HNMe₂, t-BuOH, 120° C., 2 h; (h) 1) PPh₃, I₂, imidazole, DCM, 1 h, rt; 2) H₂NCH₂CH₂NHBoc, NaOH, t-BuOH, 120° C., 3 h; 3) HCl, 1,4-dioxane, rt; (i) CH₃I, MeOH, rt, 12 h; (j) Ethyl formimidate hydrochloride, DIEA, THF, −55° C., 1 h; (k) 1) Bis-Boc-pyrazolocarboxamidine, DIEA, DCM, 2 h, rt; 2) HCl, 1,4-dioxane, rt.

### DETAILED DESCRIPTION OF INVENTION

In one preferred aspect, a variety of tryptoline-based benzothiazoles may be synthesized and used to potentiate antibiotic compounds, such as representative β-lactam antibiotics directed to MRSA. In another preferred embodiment, compound 4ad demonstrated strong RMA activity and low mammalian cytotoxicity (MRC=2 μg/mL, MIC >32 μg/mL, with GI50 >100 μg/mL). In yet another preferred embodiment, the present inventors also identified compound 12g as a novel anti-MRSA antibiotic (MRC=1 μg/mL, MIC=2 μg/mL, with GI50 of 14.1 μg/mL).

The present invention includes a new class of RMAs with a novel tryptoline-based benzothiazole scaffold. In one example, a tryptoline-based benzothiazole in this series (4ad) re-sensitizes multiple MRSA strains to cephalosporins at low concentrations (2 μg/mL) and has low mammalian cytotoxicity with a half growth inhibitory concentration (GI₅₀) >100 μg/mL in human cervical carcinoma (HeLa) cells. In addition, the present invention includes a tryptoline-based benzothiazole core scaffold which may further include various different substitutions that provide antibacterial activity against MRSA.

In one particular embodiment, the invention may include a tryptoline-based benzothiazole compound. In one particular embodiment, the tryptoline-based benzothiazole compound of the invention may be an RMA. In another particular embodiment, the tryptoline-based benzothiazole compound of the invention may be an antibiotic.

In another particular embodiment, the compound of the invention is of the formula (I):

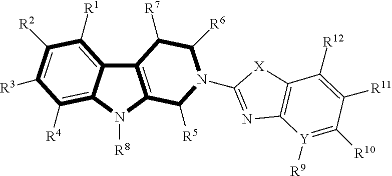

wherein formula I comprises a tryptoline compound coupled with a benzothiazole compound. In one preferred embodiment, the compound of Formula I one may form a core scaffold compound that may be further modified, and wherein such modifications may enhance RMA activity and/or antimicrobial activity, and/or decrease cytotoxicity.

In another particular embodiment, the compound of the invention is of the formula (II):

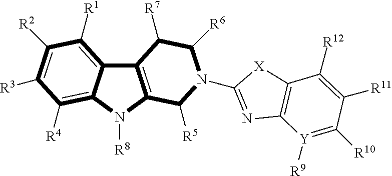

wherein,
X is independently S, O, or NMe;
Y is independently C, or N;
R1 is independently H, or Cl;
R2 is independently Cl, H, F, Br, or OMe;
R3 is independently H, or Cl;
R4 is independently H, or Cl;
R5 is independently H, or CO₂Et;
R6 is independently H, or CO₂Me;
R7 is independently H, CO₂Me, COOH, CONMe₂, CH₂OH, CH₂OCH₂OMe, CH₂SCH₂CH₂NH₃Cl, CH₂NH₂, CH₂NMe₂, CH₂N⁺Me₃I⁻, CH₂NHCH₂CH₂NH₂, CH₂NHCH═NH, (CH₂)₅NH, or CH₂NHC═NHNH₃Cl;
R8 is independently H, or Et;
R9 is independently H, Cl, or CF₃;
R10 is independently H, Cl, or CF₃;
R11 is independently Cl, H, Me, CF₃, OCF₃, NH₂, OMe, OH, OCH₂CH₂NH₂, Br, NHCOCH₂NH₃Cl, NH₂HCl, NHCH₃, and NH(CH₂)₂NH₃Cl; and
R12 is independently H, or Cl.

In another particular embodiment, the compound of the invention is of the formula (III):

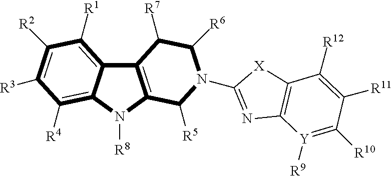

wherein,
X is independently S;
Y is independently C;
R1 is independently H;
R2 is independently Cl, or Br;
R3 is independently H;
R4 is independently H;
R5 is independently H;
R6 is independently H;
R7 is independently H, CO₂Me, COOH, CONMe₂, CH₂OH, CH₂OCH₂OMe, CH₂SCH₂CH₂NH₃Cl, CH₂NH₂, CH₂NMe₂, CH₂N⁺Me₃I⁻, CH₂NHCH₂CH₂NH₂, CH₂NHCH═NH, (CH₂)₅NH, or CH₂NHC═NHNH₃Cl;
R8 is independently H;
   R9 is independently H, and wherein R11 is selected from the group consisting of: Cl, Br, CF₃, OCF₃; or
   R9 is independently Cl, and wherein R11 is selected from the group consisting of: H, Cl, Br, NH₂, or
   R9 is independently CF₃, and wherein R11 is selected from the group consisting of: H, NH₂HCl, NHCH₃,
R10 is independently H, or CF₃; and
R12 is independently H, or Cl;
In another particular embodiment, the compound of the invention is of the formula (IV):

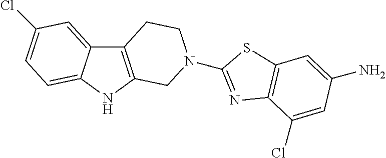 [SMILES: Nc1cc(Cl)c2nc(N3CCc4c([nH]c5ccc(Cl)cc45)C3)sc2c1]

In another particular embodiment, the compound of the invention is of the formula (V):

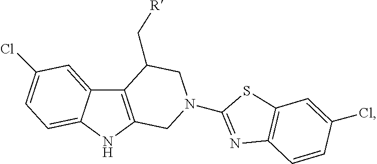

wherein R′ is NHC═NHNH₃Cl, or alternatively presented:

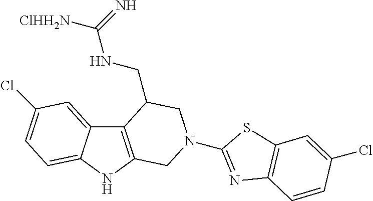

Another aspect of the invention provides an antibiotic composition comprising a compound of the invention that is capable of re-sensitizing the susceptibility of a resistant bacteria, such as methicillin-resistant S. aureus to a β-lactam antibiotic. In some embodiments, the antibiotic composition further includes a β-lactam antibiotic. Suitable β-lactam antibiotics are well known to one skilled in the art, and exemplary β-lactam antibiotics can be found in Merck Index, 15^{th} Ed., Edited by Maryadele J O'Neil, Royal Society of Chemistry, 2013, and Physicians' Desk Reference (i.e., “PDR”) 67^{th}Ed., 2013, all of which are incorporated herein by reference in their entirety. In some embodiments, the antibiotic composition comprises a tryptoline-based benzothiazole compound described herein.

The compounds of the invention can be administered to a patient or a subject to achieve a desired physiological effect. Generally, the subject is an animal, typically a mammal, and preferrably a human. The compound can be administered in a variety of forms adapted to the chosen route of administration, i.e., orally, or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal, and rectal systemic.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Typical compositions or preparations according to the invention are prepared such that an oral dosage unit form contains from about 1 to about 1000 mg of active compound. The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate, and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation. In addition to the common dosage forms set out above, the compounds of the invention may also be administered by controlled release means and/or delivery devices capable of releasing the active ingredient (prenylation inhibitor) at the required rate to maintain constant pharmacological activity for a desirable period of time Such dosage forms provide a supply of a drug to the body during a predetermined period of time and thus maintain drug levels in the therapeutic range for longer periods of time than conventional non-controlled formulations. Examples of controlled release pharmaceutical compositions and delivery devices that may be adapted for the administration of the active ingredients of the present invention are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120.548; 5,073,543; 5,639,476; 5,354,566; and 5,733,566, the disclosures of which are hereby incorporated by reference.

Pharmaceutical compositions for use in the methods of the present invention may be prepared by any of the methods of pharmacy, but all methods include the step of bringing the active ingredient into association with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

The active compound can also be administered parenterally. Solutions of the active compound as a free base or pharmacologically acceptable salt can be prepared in water suitably mixed with a surfactant such as hydroxypropylcellulose Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride Prolonged absorption of the injectable compositions of agents delaying absorption, e.g., aluminum monostearate and gelatin. Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof. The compounds of the invention can be administered to a mammal alone or in combination with pharmaceutically acceptable carriers, as noted above, the proportion of which is determined by the solubility and chemical nature of the compound, chosen route of administration and standard pharmaceutical practice.

The physician can readily determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2x to about 4-, may be required for oral administration.

The term “benzothiazole” is intended to mean a fully aromatic heteroaryl having a five-membered ring fused to a phenyl ring with the five-membered ring containing one nitrogen atom directly attached to the phenyl ring and one sulfur atom directly attached to the phenyl ring

The term “tryptoline” is intended to mean a compounds having the following general formula: 1,2,3,4-Tetrahydro-91H-pyrido[3,4-b]indole, and the following general structure:

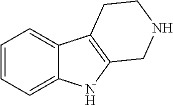

Terms “halide,” “halogen” and “halo” are used interchangeably herein and refer to fluoro, chloro, bromo, or iodo.

The term “alkyl” refers to a saturated linear monovalent hydrocarbon moiety of one to twenty, typically one to fifteen, and often one to ten carbon atoms or a saturated branched monovalent hydrocarbon moiety of three to twenty, typically three to fifteen, and often three to ten carbon atoms Exemplary alkyl group include, but are not limited to, methyl, ethyl, n-propyl, 2-propyl, tert-butyl, pentyl, iso-pentyl, hexyl, and the like.

“Alkylene” refers to a saturated linear divalent hydrocarbon moiety of one to twenty, typically one to fifteen and often one to ten carbon atoms or a branched saturated divalent hydrocarbon moiety of three to twenty, typically three to fifteen and often three to ten carbon atoms. Exemplary alkylene groups include, but are not limited to, methylene, ethylene, propylene, butylene, pentylene, and the like.

“Haloalkyl” refers to an alkyl group as defined herein in which one or more hydrogen atom is replaced by same or different halide atoms Exemplary haloalkyls include, but are not limited to, —CH₂Cl, —CF₃, —CH₂CF₃, —CH₂CCl₃, and the like

“Aryl” refers to a monovalent mono-, bi- or tricyclic aromatic hydrocarbon moiety of 6 to 15 ring atoms such as phenyl, naphthyl, etc. Aryl may be substituted with one or more, typically 1-3, and often 1 or 2 substituents. Exemplary substituents of aryl group include, but are not limited to, those substituents described for heteroaryl.

“Heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring can be substituted with one or more substituents, typically one or more, often one to four, and more often one or two substituents. Suitable substituents include alkyl, haloalkyl, heteroalkyl, heterocyclyl, halo, nitro, cyano, carboxy, acyl, -(alkylene)ₙ-COOR (where n is 0 or 1 and R is hydrogen, alkyl, optionally substituted phenylalkyl, or optionally substituted heteroaralkyl), or -(alkylene)ₙ-CONR^{a}R^{b} (where n is 0 or 1, and R^{a} and R^{b} are, independently of each other, hydrogen, alkyl, cycloalkyl, cycloalkylalkyl, hydroxyalkyl, aryl, or R^{a} and R^{b} together with the nitrogen atom to which they are attached form a heterocyclyl ring). More specifically the term heteroaryl includes, but is not limited to, pyridyl, furanyl, thiophenyl, thiazolyl, isothiazolyl, triazolyl, imidazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like. “Heterocycloalkyl” refers to a non-aromatic mono- or bicyclic moiety of three to twelve ring atoms in which one or more, typically one or two ring atoms are heteroatoms selected from N, O, or S(O)ₙ (where n is an integer from 0 to 2), the remaining ring atoms being C, where one or two C atoms can optionally be a carbonyl group. The heterocycloalkyl ring can be optionally substituted independently with one or more, typically one, two, or three, substituents. When two or more substituents are present in a heterocycloalkyl, each substituent is independently selected.

Exemplary substituents for heterocycloalkyl include, but are not limited to, alkyl, haloalkyl, heteroalkyl, halo, nitro, cyano, optionally substituted phenyl, optionally substituted heteroaryl, optionally substituted phenyalkyl, optionally substituted heteroaralkyl, acyl, -(alkylene)ₙ-COOR (n is 0 or 1 and R is hydrogen, alkyl, optionally substituted phenyl, optionally substituted phenyalkyl, or optionally substituted heteroaralkyl), or -(alkylene)ₙCONR^{a}R^{b} (where n is 0 or 1, and R^{a} and R^{b} are, independently of each other, hydrogen, alkyl, cycloalkyl, cycloalkylalkyl, hydroxyalkyl, aryl, or R and R′ together with the nitrogen atom to which they are attached form a heterocyclyl ring). More specifically the term heterocyclo includes, but is not limited to, tetrahydropyranyl, piperidino, piperazino, morpholino, thiomorpholino, thiomorpholino-1-oxide, thiomorpholino-1,1-dioxide, and the like.

“(Heterocycloalkyl)alkyl” refers to a moiety of the formula —R^{a}R^{b}, where R^{b} is heterocycloalkyl and R^{a} is alkylene as defined herein.

“Alkynyl” means a linear monovalent hydrocarbon moiety of two to ten carbon atoms or a branched monovalent hydrocarbon moiety of three to ten carbon atoms, containing at least one carbon-carbon triple bond, e.g., ethenyl, propenyl, and the like.

“Heteroalkyl” means a branched or unbranched, cyclic or acyclic saturated alkyl moiety containing carbon, hydrogen and one or more heteroatoms in place of a carbon atom, or optionally one or more heteroatom-containing substituents independently selected from =), —OR^{a}, —C(O)R^{a}, —NR^{b}R^{c}, —C(O)NR^{b}R^{c} and —S(O)ₙR^{d} (where n is an integer from 0 to 2). R^{a} is hydrogen, alkyl, haloalkyl, cycloalkyl, cycloalkylalkyl, heterocyclyl, heterocyclylalkyl, aryl, aralkyl, heteroaryl, heteroaralkyl, or acyl. R is hydrogen, alkyl, haloalkyl, cycloalkyl, cycloalkylalkyl, heterocyclyl, heterocyclylalkyl, aryl, aralkyl, heteroaryl, heteroaralkyl, or acyl. R^{c} is hydrogen, alkyl, haloalkyl, cycloalkyl, cycloalkylalkyl, heterocyclyl, heterocyclylalkyl, aryl, aralkyl, acyl, alkyl sulfonyl, carboxamido, or mono- or di-alkylcarbomoyl. Optionally, R^{b} and R^{c} can be combined together with the nitrogen to which each is attached to form a four-, five-, six- or seven-membered heterocyclic ring (e.g., a pyrrolidinyl, piperidinyl or morpholinyl ring) R^{d} is hydrogen (provided that n is 0), alkyl, haloalkyl, cycloalkyl, cycloalkylalkyl, heterocyclyl, heterocyclylalkyl, aryl, aralkyl, heteroaryl, heteroaralkyl, acyl, amino, monsubstituted amino, disubstituted amino, or hydroxyalkyl. Representative examples of heteroalkyls include, but are not limited to, 2-methoxyethyl, benzyloxymethyl, thiophen-2-ylthiomethyl, 2-hydroxyethyl, 2,3-dihydroxypropyl, and guanidine derivative of the formula —C(═NR^{a})—NR^{b}R^{c} where each of R^{a}, R^{b} and R^{c} is independently H, alkyl, cycloalkyl, heterocycloalkyl, (heterocycloalkyl)alkyl, (cycloalkyl)alkyl, and heteroalkyl.

“Acyl” refers to a moiety of the formula —C(O)R′, where R′ is alkyl, haloalkyl, aryl, or aralkyl. “Sulfonyl” refers to a moiety of the formula —S(O)₂R^{y}, where R^{y} is alkyl, haloalkyl, optionally substitute aryl, optionally substituted aralkyl, or (cycloalkyl)alkyl. “Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like, or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like, or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. The terms “pro-drug” and “prodrug” are used interchangeably herein and refer to a pharmacologically substantially inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo when they undergo solvolysis under physiological conditions or undergo enzymatic degradation Prodrug compounds of this invention may be called single, double, triple etc, depending on the number of biotransformation steps required to release the active drug within the organism, and indicating the number of functionalities present in a precursor-type form Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif. 1992). Prodrugs commonly known in the art include acid derivatives that are well known to one skilled in the art, such as, but not limited to, esters prepared by reaction of the parent acids with a suitable alcohol, or amides prepared by reaction of the parent acid compound with an amine, or basic groups reacted to form an acylated base derivative

“Protecting group” refers to a moiety, except alkyl groups, that when attached to a reactive group in a molecule masks, reduces or prevents that reactivity Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative amino or amine protecting groups include, formyl, acyl groups (such as acetyl, trifluoroacetyl, and benzoyl), benzyl, alkoxycarbonyl (such as benzyloxycarbonyl (CBZ), and tert-butoxycarbonyl (Boc)), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), sulfonyl, and the like.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc, of the mammal to be treated. “Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. “subject”

When describing a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. As used herein, the terms “those defined above” and “those defined herein” when referring to a variable incorporates by reference the broad definition of the variable as well as any narrow definitions, if any.

As used herein, the following abbreviations are defined as: MRSA, methicillin-resistant S. aureus; RMA, resistance-modifying agent; MSSA, methicillinsensitive S. aureus; CLSI, Clinical Laboratory Standards Institutes; SAR, structure-activity relationship; MIC, minimal inhibitory concentration; MRC, minimum resensitizing concentration; GI₅₀, half growth inhibitory concentration; HeLa cells, human cervical carcinoma cells; HAIs, hospital acquired infections; CLSI, Clinical & Laboratory Standards Institute; DMSO, dimethyl sulfoxide; TFA, trifluoroacetic acid; Rt, LCMS retention time; DIEA, N,N-Diisopropylethylamine.

The invention is further described with reference to the following non-limiting examples.

### EXAMPLES

### Example 1. Chemistry of Tryptoline-Based Benzothiazoles

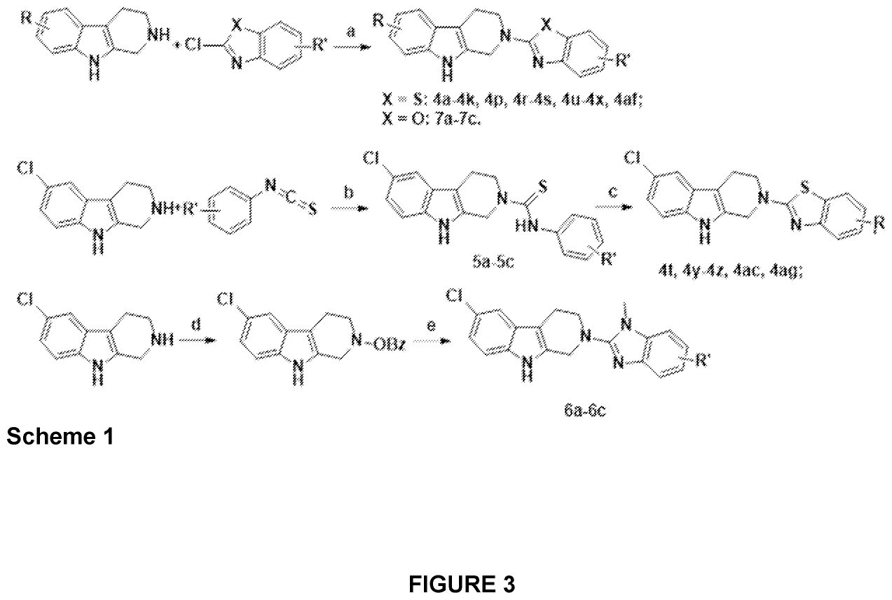

Tryptoline-based benzothiazoles were either prepared by a one-step reaction using 2-chlorobenzothiazoles and tryptoline as reactants under basic conditions (4a-4k, 4p, 4r-4s, 4u-4x, 4af, Scheme 1), or by a two-step method, which included the synthesis of thiourea (5a-5c, Scheme 1) and palladium catalyzed cyclization of thiourea (4t, 4y-4z, 4ac and 4ag, Scheme 1). The synthesis of tryptoline-based benzoxazoles also followed this method (7a-7c, Scheme 1). For the synthesis of tryptoline-based benzimidazoles, the present inventors used copper-catalyzed amination of benzimidazole. The tryptoline was transformed into O-acylhydroxylamine, and then reacted with N-methyl benzimidazole using C—H zincation/copper-catalyzed electrophilic amination (6a-6c, Scheme 1; see FIG. 3).

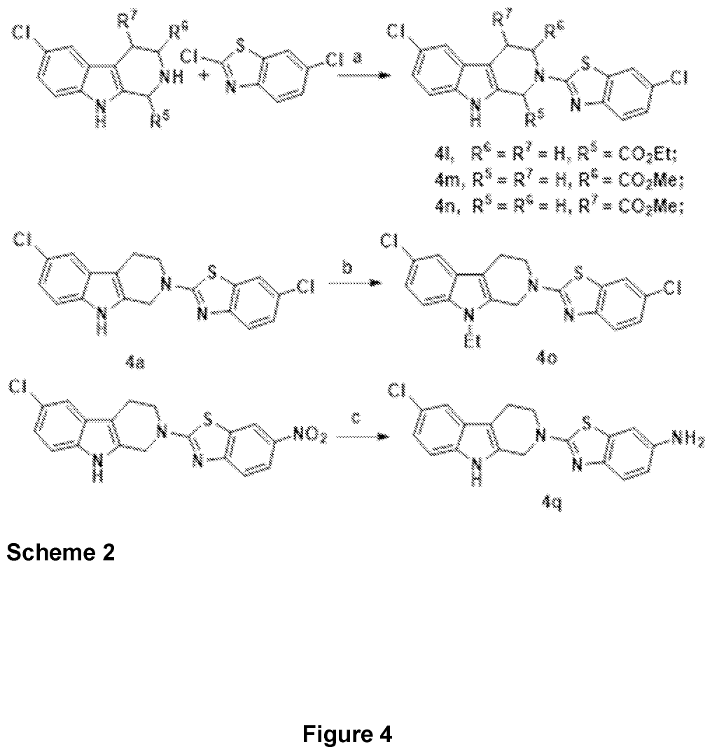

The tryptoline cores with ester group at different positions (R⁵, R⁶ and R⁷) reacted with 2, 6-dichlorobenzo[d]thiazole affording 4l-4n (Scheme 2). Replacement of indole nitrogen proton of 4a with ethyl group gave compound 4o (Scheme 2), and compound 4q was prepared from reduction of its nitro precursor (Scheme 2; see FIG. 4).

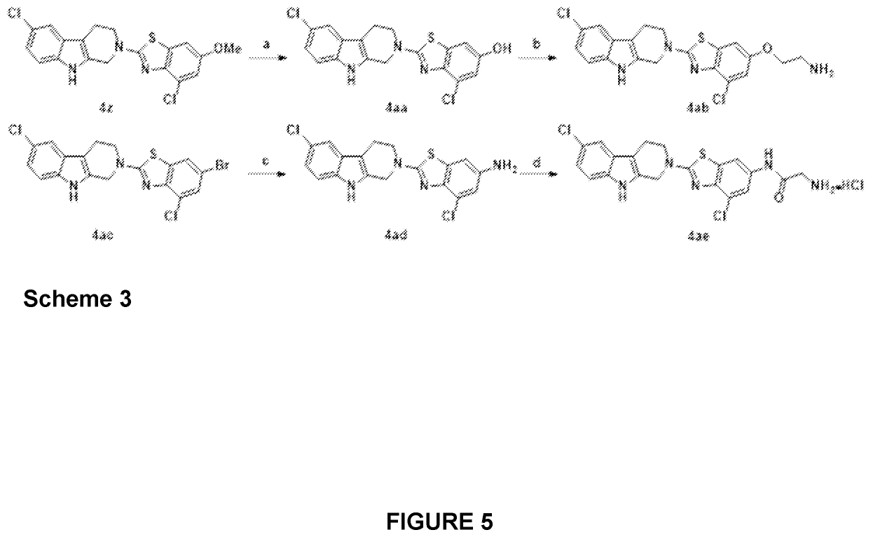

De-protection of methoxy group of 4z with BBr₃ afforded 4aa, which was further functionalized into amine 4ab (Scheme 3). By using copper catalyzed amination, 4ac was transformed into 4ad, which was coupled with amino acid Boc-Gly-OH giving compound 4ae after the removal of N-Boc group (Scheme 3; see FIG. 5).

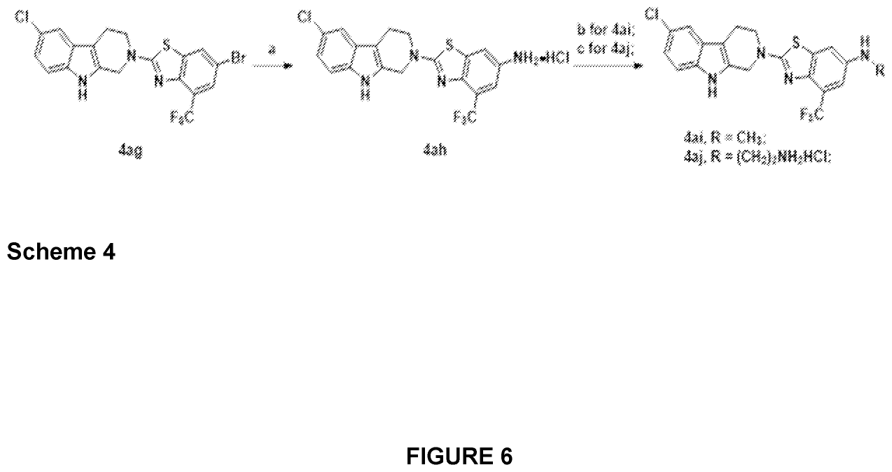

Compound 4ah was also prepared by copper catalyzed amination of compound 4ag (Scheme 4; see FIG. 6). Further reductive amination of 4ah gave 4ai and 4aj. (Scheme 4; see FIG. 6).

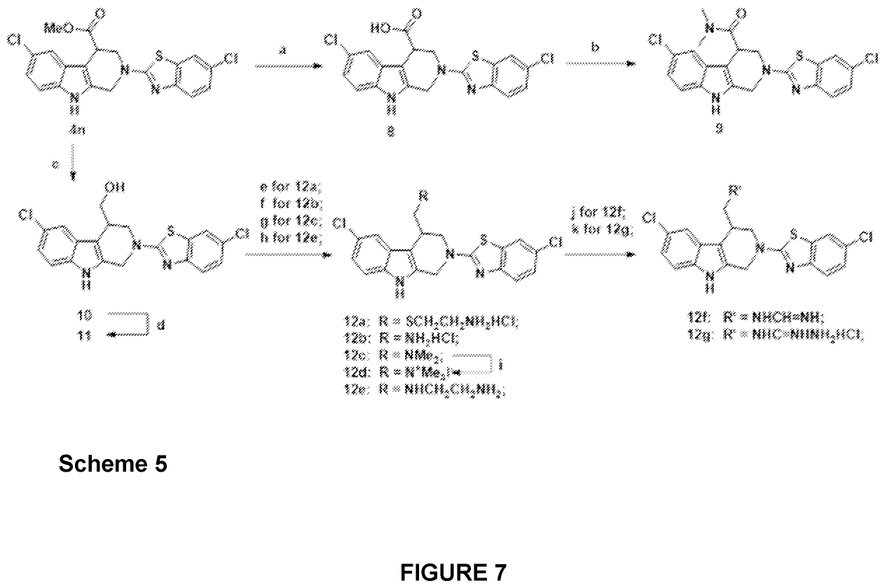

Various functional groups, such as amide, alcohol, amine and thioether were introduced to R⁷ position to probe the SAR and to optimize the physiochemical properties. All these chiral series prepared here were racemic mixture. As show in Scheme 5, hydrolysis of the ester 4n afforded free acid 8, and then amide coupling with dimethylamine afforded amide 9. Reduction of the ester 4n with LiAlH₄ provided the alcohol 10, and further reaction with MOM chloride afforded 11. Compound 12a-12e were prepared via the corresponding iodide intermediate. Further functionalization of the primary amine afforded compound 12d, 12f and 12g (Scheme 5; see FIG. 7).

### Example 2. Structure Activity Relationship (SAR) Study

RMA activity of the analogues was tested by assessing their abilities to sensitize MRSA to the antibiotic cefazolin (a first-generation cephalosporin). The well characterized strain MRSA ATCC BAA-44 was tested as previously described. The minimum re-sensitizing concentration (MRC) was defined as the concentration of analogues at which no overnight growth was observed in the presence of the CLSI breakpoint for antibiotic sensitivity (8 μg/mL for cefazolin). The minimal inhibitory concentration (MIC), or the lowest concentration at which S. aureus is considered susceptible to an antibacterial, was determined by the standard broth microdilution method detailed in the CLSI handbook. RMA activity was compared by the ratio of MIC/MRC. The half growth inhibitory concentration (GI₅₀) of each analogue against HeLa cells was determined as previously described. Compounds that displayed improved RMA activity relative were then tested for toxicity against the growth of human cervical adenocarcinoma (HeLa) cells by incubating different concentrations of each compound with cells for 24 h and assessing viability at each concentration using the Cell Titer Glo mammalian viability assay (Promega). The luminescence of each sample was recorded in an Envision Multilabel Plate Reader (Perkin Elmer). Results of MICs and MRCs were confirmed by testing in triplicate. The GI₅₀ assay was performed in duplicate. Cefazolin and Cefuroxime were used as antibiotic controls and inhibited the growth of MRSA BAA-44 on average at 128 μg/mL or 256 μg/mL for both Cefazolin and Cefuroxime.

### Example 3. Initial Screening of Tryptoline-Based Structure for the RMA Activity

For the initial screening, the present inventors chose chloro-substituted tryptoline as a core structure based on previous studies, and found that tryptoline with chloro-substituted benzothiazole motif 4a showed good RMA activity (32 folds, entry 1, Table 1), while benzothiazole without Cl-substitution 4b (entry 2, Table 1) or with methyl substitution 4c (entry 3, Table 1) gave no RMA activity. When the benzothiazole motif was replaced by [1,3]thiazolo[4,5-b]pyridine 4d or [1,3]thiazolo[4,5-b]pyridine with CF₃ substitution 4e, the RMA activity was abolished (entries 4 and 5, Table 1). Interestingly, compounds with a thiourea motif (5a-5c, entries 6-8, Table 1), similar structure to benzothiazole, showed low RMA activity (2-fold), while giving good antibacterial activity instead by themselves (MIC=2 μg/mL). Surprisingly, compounds with benzimidazole motif (6a-6c, entries 9-11, Table 1) or with benzoxazole motif (7a-7c, entries 12-14, Table 1) had no RMA activity. The SAR studies of tryptoline-based structure revealed that benzothiazole core motif has the most optimal RMA activity and the thiourea motif has good antibacterial activity and moderate RMA activity.

### Example 4. Optimization of Tryptoline Motif for RMA Activity

In this embodiment, the SAR study continued with compound 4a (entry 1, Table 1). First, the present inventors kept chloro-substituted benzothiazole motif and explored the SAR with various substituted tryptolines. RMA activity was lost when the R² chloride was moved to a different position on the substituted tryptoline (R¹, R³ and R⁴, 4f-4h, entries 2-4, Table 2). Other substitutions on the R² position also led to a decrease or abolition of RMA activity (F, Br and OMe, 4i-4k, entries 5-7, Table 2). Furthermore, additional substitutions on the R⁵, R⁶ or R⁸ position (CO₂Et for 4l, CO₂Me for 4m, Et for 4o, entries 8, 9 and 11, Table 2) with Cl on R² position maintained also abolished the RMA activity. However, analog with R⁷—CO₂Me has similar RMA activity to the parent compound 4a (32 folds, 4n, entry 10, Table 2) indicating that modifications on this position might be tolerated. These SAR studies show that the nature of substituents and their substitution position on benzene ring of tryptoline both play crucial roles in retaining RMA activity, and chloride at R² position is essential for good RMA activity. As used herein the term essential in this context means most preferred, or most effective for the intended activity of the compound. Finally, we discovered that substitutions on indole nitrogen or R⁵ and R-piperidine substitutions led to loss of the RMA activity, while the R⁷-piperidine substitution was tolerated for RMA activity.

### Example 5. Optimization of Benzothiazole Motif for RMA Activity

After optimizing the tryptoline core, the present inventors next attempted to improve the RMA activity and lower toxicity by modifying benzothiazole motif. First, the present inventors explored the chloride replacements on the R¹¹ of benzothiazole motif. While bromide analog 4p retained the RMA activity, the corresponding NH₂— and CO₂Et-substituted analogs 4q-4r had a complete loss of RMA activity. Interestingly, the CF₃-substituted analog 4s had a 4 fold increase of RMA activity relative to 4a (entry 5, Table 3), however, the GI₅₀ (HeLa) decreased from 13.2 μg/mL to 4.6 μg/mL. The OCF₃— analog 4t lost the RMA activity completely, while possessing great antibacterial activity by itself (MIC=1 ug/mL). Moving the chloride from R¹¹ to different positions led to various degrees of RMA activities: R⁹ analog 4w maintained similar RMA activity; R¹⁰ analog 4v showed no RMA activity; R¹² analog 4u displayed reduced RMA activity and increased MIC (entries 7-9, Table 3). R⁹—CF₃ analog 4af gave similar RMA activity to R⁹—Cl analog 4w with 3 fold worse GI₅₀ (entry 16, Table 3). Next, the present inventors prepared a series of compounds with either Cl or CF₃ on R⁹ while varying R¹¹ substitutions (Cl: 4x-4ae, entries 10-17; CF₃: 4af-4aj, entries 18-22, Table 3). The R¹¹ substituents covered a wide range, including halide, hydroxyl, ether, amine and amide. While three analogs (4ad, 4af, 4ai) displayed good RMA activities, and several (4x, 4y, 4ae, 4aj) showed excellent MICs. Compound 4ad with Cl on R⁹ and NH₂ on R¹¹ stands out due to its great RMA activity and low mammalian cytotoxicity (GI₅₀>100 μg/mL, the highest concentration tested, entry 16, Table 3).

### Example 6. Further Optimization of Tryptoline Motif on R7 for the RMA Activity

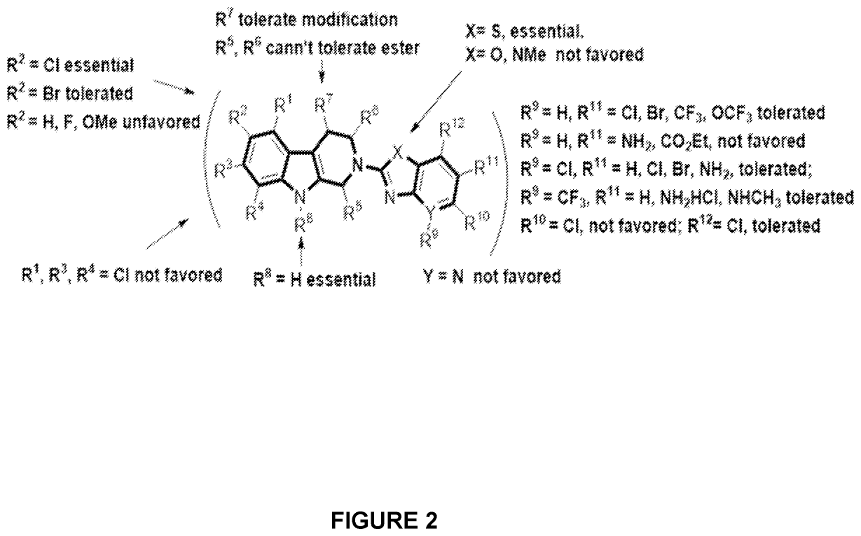

During the preliminary SAR studies of tryptoline motif core, we found that R⁷ position can tolerate the methyl ester substitution without compromising the RMA activity (4n, entry 1, Table 4). Further SAR exploration at this position was then performed. The introduction of carboxylic acid and amide significantly decreased or abolished the RMA activity (8 and 9, entries 2-3, Table 4). Surprisingly, conversion of the ester 4n into alcohol 10 and ether 11 led to antibacterial activity with good MIC (MIC=2.0 μg/mL, entry 4-5, Table 4). Additional analogs incorporating basic amines, charged quarternary ammonium salt, amidine and guarnidine etc. (12a-12g) resulted in good to excellent MICs by themselves, therefore masking their potential RMA activities. Among them, quarternary ammonium salt analog 12d and guarnidine analog 12g showed relatively low level of mammalian cytotoxicity (GI₅₀ of 17.8 and 14.1 ug/mL respectively). While these R⁷-substituted analogs showed diminished RMA activities, their potent antibacterial activities combined with improved physicochemical properties offer promise for the discovery of novel antibacterial agents. On the basis of the findings and analysis described above, a summary of the SAR of tryptoline-based benzothiazoles in this study is illustrated in FIG. 2.

We next explored the scope of RMA activity of 4ad in different MRSA strains, including MRSA BAA-1683, MRSA BAA-1764, MRSA NR-46411, NRS-384 and NRS-100. These strains were selected because of their diverse geographical origins, genetic background, and resistance profile. We also tested RMA activity in combination with another commercial antibiotic, cefuroxime, which is a second-generation cephalosporin. MRCs in the presence of cefuroxime were determined using the same method as those for cefazolin. Our most potent compound 4ad potentiates both antibiotic cefazolin and cefuroxime in the five MRSA strains tested here (Table 5).

Furthermore, we explored the scope of the antibacterial activity of 12g in a panel of MRSA and MSSA strains including BAA-44 (MIC=1 μg/mL), MRSA-252 (MIC=2 μg/mL), B. subtilis NR-607 (MIC=2 μg/mL), E. faecium HM-460 (MIC=4 μg/mL), E. faecium 28977 (MIC=2 μg/mL) and MSSA (MIC=0.5 μg/mL). Analogue 12g displayed good antibacterial activities (MIC range from 0.5 μg/mL to 4 μg/mL) for all six MRSA and MSSA strains tested here, despite the resistance profiles.

### Example 7. Materials and Experimental Design

All reagents were obtained commercially and used without further purification unless otherwise noted. MRSA strain ATCC BAA-44 was a gift from the laboratory of Daniel Feldheim. Strains BAA-1683(MRSA), BAA-1764(MRSA), NR-46411(MRSA), NRS-100(MRSA), MRSA-252, B.subtilis NR-607, E. faecium HM-460, E. faecium 28977, MSSA, NRS-384(MRSA) and HeLa cells were purchased from ATCC (http://www.atcc.org). CellTiter-Glo® luminescent cell viability assay kit was purchased from Promega Corp. Thin-layer chromatography (TLC) analysis of reaction mixtures was performed on Dynamicadsorbents silica gel F-254 TLC plates. Flash chromatography was carried out on Zeoprep 60 ECO silica gel. ¹H NMR spectra were recorded with Varian INOVA (400, 500 MHz) and Bruker spectrometers. Mass spectral and analytical data were obtained via the PE SCIEX/ABI API QSTAR Pulsar iHybrid LC/MS/MS (Applied Biosystems) operated by the Central Analytical Laboratory, University of Colorado at Boulder. All compounds were evaluated for purity by using an Agilent 1260 series HPLC system coupled with a 6120 Quadrupole mass spectrometer (column: ZORBAX Narrow Bore SB-C18 RRHT, 2.1×50 mm, 1.8 μm, PN 827700-902) with a minimum purity standard of ≥90%. The system was eluted at 0.35 mL/min with a gradient of water/acetonitrile with 0.1% formic acid: 0-5 min, 5-95% acetonitrile; 5-7 min, 95% acetonitrile; 7-7.25 min, 95-5% acetonitrile; 7.25-12 min, 5% acetonitrile.

### Example 8. General Procedure of the Synthesis of Tryptoline-Based Benzothiazoles

Method A: To a round bottom flask was added tryptoline (21 mg, 0.100 mmol), 2-chlorobenzothiazole (31 mg, 0.150 mmol), K₂CO₃ (70 mg, 0.500 mmol) and DMF (2.0 mL), and the reaction mixture was stirred for 12 h at 110° C. After the reaction was then cooled to room temperature, ethyl acetate (10 mL) and H₂O (10 mL) were added into the mixture, and the aqueous layer was extracted with 2×10 mL ethyl acetate. The organic layer then was combined and washed with brine and dried over Na₂SO₄. The solvents were removed under vacuum, and the residue was purified by flash chromatography on silica gel using ethyl acetate/hexane as eluent.

Method B: Synthesis of thiourea: To a round bottom flask was added tryptoline (206 mg, 1.00 mmol), isothiocyanate (1.10 mmol) and DCM (10 mL), and the reaction mixture was stirred at room temperature for 2 h. The solvents were removed under vacuum and the residue was purified by silica gel flash chromatography using ethyl acetate/hexane (1:2, v/v) as eluent. Pd-catalyzed cyclization of thiourea: To a flame-dried 150×20 mm reaction tube was added thiourea (0.500 mmol), activated manganese dioxide (0.0043 g, 0.050 mmol), and tetrakistriphenylphosphine palladium(0) (17.3 mg, 0.015 mmol). Anhydrous acetonitrile (6 mL) was then added. The reaction system was then heated to reflux (80° C.) with vigorous stirring under an oxygen atmosphere for 6 h. The reaction mixture was then cooled to room temperature and filtered to remove solid manganese dioxide. The solvents were removed under vacuum and the residue was then purified via silica gel column chromatography using ethyl acetate/hexane (1:3, v/v) as eluent.

In another particular embodiment, the invention may include the compound: 6-Chloro-2-(6-chloro-1,3-benzothiazol-2-yl)-2,3,4,9-tetrahydro-1H-pyrido[3,4-b]indole (4a) prepared by method A. Pale yellow solid was obtained with a yield of 38%. ¹H NMR (300 MHz, DMSO-d₆) δ 11.15 (s, 1H), 7.91 (q, J=2.5 Hz, 1H), 7.57-7.41 (m, 2H), 7.41-7.21 (m, 2H), 7.06 (dt, J=8.7, 2.4 Hz, 1H), 4.87 (s, 2H), 3.92 (d, J=5.6 Hz, 2H), 2.86 (d, J=6.0 Hz, 2H). ¹³C NMR (101 MHz, DMSO-d₆) δ 168.73, 151.24, 134.47, 132.22, 131.86, 127.55, 126.23, 125.04, 123.32, 120.96, 120.86, 119.55, 117.06, 112.63, 106.70, 46.99, 45.62, 20.28. LC-MS, Rt=7.483 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 2-(1,3-Benzothiazol-2-yl)-6-chloro-2,3,4,9-tetrahydro-1H-pyrido[3,4-b]indole (4b) was prepared by method A. Pale yellow solid was obtained with a yield of 48%. ¹H NMR (400 MHz, Chloroform-d) S 7.97 (s, 1H), 7.63-7.59 (m, 1H), 7.56 (d, J=7.9 Hz, 1H), 7.45 (d, J=2.0 Hz, 1H), 7.30 (td, J=8.3, 7.8, 1.3 Hz, 1H), 7.25-7.24 (m, (s, 1H), 7.14-7.04 (m, 1H), 4.93 (s, 2H), 3.94 (t, J=5.7 Hz, 2H), 2.95 (t, J=5.8 Hz, 2H). LC-MS, Rt=7.397 min, [M+H]⁺=340.0. HRMS (ESI) m/z calcd. for C₁₈H₁₅ClN₃S [M+H]⁺=340.0670, found 340.0671.

In another particular embodiment, the invention may include the compound: 6-Chloro-2-(6-methyl-1,3-benzothiazol-2-yl)-2,3,4,9-tetrahydro-1H-pyrido[3,4-b]indole (4c) prepared by method A. Yellow solid was obtained with a yield of 45%. ¹H NMR (400 MHz, DMSO-d₆) δ 11.14 (s, 1H), 7.51 (d, J=1.7 Hz, 1H), 7.42 (d, J=2.0 Hz, 1H), 7.32 (dd, J=8.4, 3.8 Hz, 2H), 7.03 (ddd, J=13.0, 8.4, 1.9 Hz, 2H), 4.80 (s, 2H), 3.84 (t, J=5.7 Hz, 2H), 2.80 (t, J=5.7 Hz, 2H), 2.28 (s, 3H). ¹³C NMR (101 MHz, DMSO-d₆) δ 167.57, 150.20, 134.47, 132.51, 130.55, 130.37, 127.61, 127.11, 123.30, 121.13, 121.05, 120.82, 118.37, 118.30, 117.08, 117.00, 112.65, 106.71, 46.94, 45.57, 20.84, 20.78, 20.29. LC-MS, Rt=7.881 min, [M+H]+=354.0. HRMS (ESI) m/z calcd. for C₁₉H₁₇ClN₃S [M+H]⁺=354.0827, found 354.0840.

In another particular embodiment, the invention may include the compound: 6-Chloro-2-([1,3]thiazolo[4,5-b]pyridin-2-yl)-2,3,4,9-tetrahydro-1H-pyrido[3,4-b]indole (4d) prepared by method A. Yellow solid was obtained with a yield of 29%. ¹H NMR (500 MHz, DMSO-d₆) δ 11.22 (s, 1H), 8.32 (dd, J=4.8, 1.7 Hz, 1H), 8.22 (dd, J=7.8, 1.7 Hz, 1H), 7.50 (d, J=2.1 Hz, 1H), 7.37 (d, J=8.6 Hz, 1H), 7.11-6.83 (m, 2H), 4.94 (s, 2H), 3.99 (t, J=4.4 Hz, 2H), 2.90 (t, J=5.8 Hz, 2H). LC-MS, Rt=5.552 min, [M+H]⁺=341.0. HRMS (ESI) m/z calcd. for C₁₇H₁₄ClN₄S [M+H]⁺=341.0623, found 341.0658.

In another particular embodiment, the invention may include the compound: 6-Chloro-2-[6-(trifluoromethyl)[1,3]thiazolo[4,5-b]pyridin-2-yl]-2,3,4,9-tetrahydro-1H-pyrido[3,4-b]indole (4e) prepared by method A. Pale yellow solid was obtained with a yield of 39%. ¹H NMR (500 MHz, Chloroform-d) δ 8.73-8.61 (m, 1H), 8.12 (d, J=2.2 Hz, 1H), 7.49-7.43 (m, 1H), 7.47-7.33 (m, 1H), 7.21 (ddd, J=12.9, 8.7, 2.0 Hz, 1H), 5.14 (d, J=24.3 Hz, 2H), 4.04 (d, J=9.8 Hz, 2H), 2.98 (dddd, J=12.4, 6.4, 4.2, 1.9 Hz, 2H). LC-MS, Rt=6.626 min, [M+H]⁺=408.0. HRMS (ESI) m/z calcd. for C₁₈H₁₃ClF₃N₄S [M+H]⁺=409.0496, found 409.0448.

In another particular embodiment, the invention may include the compound: 6-Chloro-N-(4-chlorophenyl)-1,3,4,9-tetrahydro-2H-pyrido[3,4-b]indole-2-carbothioamide (5a) prepared by method B. Pale yellow solid was obtained with a yield of 63%. ¹H NMR (400 MHz, Chloroform-d) δ 8.05 (s, 1H), 7.40 (d, J=2.0 Hz, 1H), 7.36 (s, 1H), 7.27 (d, J=8.6 Hz, 2H), 7.20 (d, J=8.5 Hz, 1H), 7.15 (d, J=8.7 Hz, 2H), 7.10 (dd, J=8.6, 2.0 Hz, 1H), 5.05 (s, 2H), 4.12-4.02 (m, 2H), 2.82 (t, J=5.9 Hz, 2H). LC-MS, Rt=7.246 min, [M+H]⁺=376.0. HRMS (ESI) m/z calcd. for C₁₈H₁₆Cl₂N₃S [M+H]⁺=376.0437, found 376.0462.

In another particular embodiment, the invention may include the compound: 6-Chloro-N-[4-(trifluoromethoxy)phenyl]-1,3,4,9-tetrahydro-2H-pyrido[3,4-b]indole-2-carbothioamide (5b) prepared by method B. Pale yellow solid was obtained with a yield of 59%. ¹H NMR (400 MHz, DMSO-d₆) δ 11.17 (s, 1H), 9.64 (s, 1H), 7.49 (d, J=2.1 Hz, 1H), 7.45 (s, 1H), 7.43 (s, 1H), 7.34 (d, J=8.6 Hz, 1H), 7.34-7.29 (m, 1H), 7.32-7.27 (m, 1H), 7.05 (dd, J=8.6, 2.1 Hz, 1H), 5.18 (s, 2H), 4.22 (t, J=5.6 Hz, 2H), 2.85 (t, J=5.5 Hz, 2H). ¹³C NMR (101 MHz, DMSO-d₆) δ 181.91, 144.74, 144.72, 140.29, 134.59, 133.08, 127.58, 126.96, 123.30, 121.45, 120.81, 120.74, 118.91, 117.08, 112.60, 109.60, 107.16, 47.19, 46.55, 40.15, 39.94, 39.73, 39.52, 39.31, 39.10, 38.89, 20.83. LC-MS, Rt=6.354 min, [M+H]⁺=426.0. HRMS (ESI) m/z calcd. for C₁₉H₁₆ClF₃N₃OS [M+H]⁺=426.0650, found 426.0690.

In another particular embodiment, the invention may include the compound: 6-Chloro-N-[3-(trifluoromethyl)phenyl]-1,3,4,9-tetrahydro-2H-pyrido[3,4-b]indole-2-carbothioamide (5c) prepared by method B. Pale yellow solid was obtained with a yield of 60%. ¹H NMR (400 MHz, Chloroform-d) δ 7.86 (s, 1H), 7.59 (d, J=8.4 Hz, 2H), 7.44 (dd, J=4.2, 2.0 Hz, 1H), 7.36-7.27 (m, 3H), 7.25-7.24 (m, 3H), 7.16-7.09 (m, 1H), 5.10 (m, 2H), 4.21-4.03 (m, 2H), 2.89 (t, J=6.0 Hz, 21H). LC-MS, Rt=7.520 min, [M+H]⁺=410.0. HRMS (ESI) m/z calcd. for C₁₉H₁₆ClF₃N₃S [M+H]⁺=410.0701, found 410.0694.

### Example 9. Specific Procedures for the Synthesis of Select Tryptoline-Based Benzimidazole Compositions

To a round bottom flask was added tryptoline (206 mg, 1.00 mmol), BPO (242 mg, 1.00 mmol), Na₂HPO₄ (215 mg, 1.50 mmol) and DMF (10 mL), and the reaction mixture was stirred for 12 h at room temperature. Ethyl acetate (50 mL) and H₂O (50 mL) were added into the mixture, and the aqueous layer was extract with 2×50 mL ethyl acetate. The organic layer then was combined and washed with saturated NaHCO₃solution and then with brine and dried over Na₂SO₄. The solvents were removed under vacuum, and the residue was purified by silica gel flash chromatography using ethyl acetate/hexane (1:2, v/v) as eluent. O-acylhydroxylamine was obtained as pale white solid with a yield of 26%. To a 10 mL tube charged with N-methylbenzimidazole (27 mg, 0.200 mmol, 1.0 equiv) was added THF (1 mL) followed by dropwise addition of (TMP)ZnCl.LiCl solution (0.200 mmol, 1.0 equiv) under N₂. The resulting mixture was stirred vigorously at room temperature for 1 h. Then a mixture of Cu(OAc)₂ (3.70 mg, 0.020 mmol, 0.10 equiv) and O-acylhydroxylamine (0.240 mmol, 1.2 equiv) in THF (1 mL) was added dropwise to the heteroarylzinc mixture under N₂. Upon complete consumption of the N-methylbenzimidazole, the reaction was quenched by dropwise addition of a saturated NH₄Cl solution (1 mL). The reaction mixture was subsequently basified with saturated Na₂CO₃ solution (5 mL) and extracted with ethyl acetate (3×5 mL). The combined organic layers were washed with brine (5 mL), dried over Na₂SO₄, and filtered. The filtrate was concentrated under reduced pressure. The crude reaction mixture was purified by silica gel flash-column chromatography.

In another particular embodiment, the invention may include the compound: 6-Chloro-2-(1-methyl-1H-benzimidazol-2-yl)-2,3,4,9-tetrahydro-1H-pyrido[3,4-b]indole (6a) prepared by the general procedure. Pale yellow solid was obtained with a yield of 39%. ¹H NMR (400 MHz, Chloroform-d) δ 8.79 (s, 1H), 7.60-7.55 (m, 1H), 7.45 (d, J=2.1 Hz, 1H), 7.24 (m, 1H), 7.23-7.16 (m, 3H), 7.07 (dd, J=8.6, 2.1 Hz, 3H), 4.67 (s, 2H), 3.70 (s, 3H), 3.60 (t, J=5.6 Hz, 2H), 2.98 (t, J=5.6 Hz, 2H). LC-MS, Rt=4.719 min, [M+H]⁺=353.1. HRMS (ESI) m/z calcd. for C₁₉H₁₇ClN₄ [M+H]⁺=337.1215, found 337.1198.

In another particular embodiment, the invention may include the compound: 6-Chloro-2-(6-chloro-1-methyl-1H-benzimidazol-2-yl)-2,3,4,9-tetrahydro-1H-pyrido[3,4-b]indole (6b) prepared by the general procedure. Pale yellow solid was obtained with a yield of 38%. ¹H NMR (500 MHz, Chloroform-d) δ 8.11 (s, 1H), 7.57 (d, J=1.8 Hz, 1H), 7.50 (dd, J=8.4, 2.1 Hz, 2H), 7.28 (s, 13H), 7.22-7.05 (m, 1H), 4.65 (s, 2H), 3.71 (s, 3H), 3.64 (t, J=5.7 Hz, 2H), 3.01 (t, J=5.8 Hz, 2H). LC-MS, Rt=5.919 min, [M+H]⁺=371.0. HRMS (ESI) m/z calcd. for C₁₉H₁₇Cl₂N₄ [M+H]⁺=371.0825, found 371.0862.

In another particular embodiment, the invention may include the compound: 6-Chloro-2-[1-methyl-6-(trifluoromethyl)-1H-benzimidazol-2-yl]-2,3,4,9-tetrahydro-1H-pyrido[3,4-b]indole (6c) prepared by the general procedure. Pale yellow solid was obtained with a yield of 36%. ¹H NMR (400 MHz, Chloroform-d) δ 9.17 (d, J=12.5 Hz, 1H), 7.83-7.74 (m, 1H), 7.51-7.37 (m, 3H), 7.32-7.24 (m, 1H), 7.12 (ddd, J=8.7, 1.5, 0.6 Hz, 1H), 7.04 (ddd, J=8.6, 2.0, 1.3 Hz, 1H), 4.77-4.57 (m, 2H), 3.73 (d, J=3.9 Hz, 3H), 3.65 (dt, J=8.4, 5.6 Hz, 2H), 3.02-2.90 (m, 2H). LC-MS, Rt=6.748 min, [M+H]⁺=405.1. HRMS (ESI) m/z calcd. for C₂₀H₁₇ClF₃N₄ [M+H]⁺=405.1089, found 405.1087.

In another particular embodiment, the invention may include the compound: 2-(1,3-Benzoxazol-2-yl)-6-chloro-2,3,4,9-tetrahydro-1H-pyrido[3,4-b]indole (7a) prepared following method A by using 2-chlorobenzoxazole. Pale yellow solid was obtained with a yield of 38%. ¹H NMR (400 MHz, Chloroform-d) δ 8.12 (s, 1H), 7.45 (d, J=2.0 Hz, 1H), 7.36 (dt, J=7.8, 0.7 Hz, 1H), 7.30 (d, J=1.0 Hz, 1H), 7.24 (m, 1H), 7.17 (td, J=7.7, 1.1 Hz, 1H), 7.11 (dd, J=8.6, 2.1 Hz, 1H), 7.04 (td, J=7.7, 1.3 Hz, 1H), 4.91 (s, 2H), 4.06 (t, J=5.7 Hz, 2H), 2.93 (dd, J=6.7, 5.1 Hz, 2H). LC-MS, Rt=7.746 min, [M+H]⁺=358.0. HRMS (ESI) m/z calcd. for C₁₈H₁₅ClN₃₀ [M+H]⁺=324.0899, found 324.0901.

In another particular embodiment, the invention may include the compound: 6-Chloro-2-(6-chloro-1,3-benzoxazol-2-yl)-2,3,4,9-tetrahydro-1H-pyrido[3,4-b]indole (7b) prepared following method A by using 2,6-dichloro-benzoxazole. Pale yellow solid was obtained with a yield of 39%. ¹H NMR (400 MHz, Methanol-d₄) δ 7.40 (dd, J=2.1, 0.6 Hz, 1H), 7.38-7.31 (m, 1H), 7.29 (ddd, J=7.8, 1.3, 0.6 Hz, 1H), 7.25 (d, J=0.6 Hz, 1H), 7.17 (td, J=7.7, 1.2 Hz, 1H), 7.10-6.98 (m, 2H), 4.88 (s, 1H), 4.05 (t, J=5.8 Hz, 2H), 2.90 (t, J=5.7 Hz, 2H). LC-MS, Rt=7.284 min, [M+H]⁺=324.1. HRMS (ESI) m/z calcd. for C₁₈H₁₄Cl₂N₃₀ [M+H]⁺=358.0509, found 358.0535.

In another particular embodiment, the invention may include the compound: 6-Chloro-2-[6-(trifluoromethyl)-1,3-benzoxazol-2-yl]-2,3,4,9-tetrahydro-1H-pyrido[3,4-b]indole (7c) prepared following method A by using 2-chloro-6-(trifluoromethyl)-benzoxazole. Pale yellow solid was obtained with a yield of 35%. ¹H NMR (500 MHz, Chloroform-d) δ 8.11 (s, 1H), 7.49-7.40 (m, 2H), 7.25 (d, J=8.6 Hz, 1H), 7.16-7.06 (m, 2H), 4.94 (s, 2H), 4.09 (t, J=5.8 Hz, 2H), 2.91 (t, J=5.8 Hz, 2H). LC-MS, Rt=7.928 min, [M+H]⁺=392.0. HRMS (ESI) m/z calcd. for C₁₉H₁₄ClF₃N₃₀ [M+H]⁺=392.0773, found 392.0739.

In another particular embodiment, the invention may include the compound: 5-Chloro-2-(6-chloro-1,3-benzothiazol-2-yl)-2,3,4,9-tetrahydro-1H-pyrido[3,4-b]indole (4M) prepared by method A. Yellow solid was obtained with a yield of 46%. ¹H NMR (500 MHz, Chloroform-d) δ 9.29 (s, 1H), 7.61 (d, J=2.1 Hz, 1H), 7.47 (d, J=8.6 Hz, 1H), 7.41 (d, J=2.1 Hz, 1H), 7.26 (dd, J=8.5, 2.0 Hz, 1H), 7.07 (s, 1H), 7.05 (s, 1H), 4.95 (s, 2H), 3.99-3.90 (m, 2H), 3.37 (td, J =5.6, 4.7, 2.8 Hz, 2H).). LC-MS, Rt=7.013 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 7-Chloro-2-(6-chloro-1,3-benzothiazol-2-yl)-2,3,4,9-tetrahydro-1H-pyrido[3,4-b]indole (4g) prepared by method A. Yellow solid was obtained with a yield of 48%. ¹H NMR (400 MHz, Chloroform-d) δ 7.94 (s, 1H), 7.57 (d, J=2.2 Hz, 1H), 7.44 (d, J=8.6 Hz, 1H), 7.38 (d, J=8.4 Hz, 1H), 7.32 (dd, J=1.9, 0.6 Hz, 1H), 7.24 (m, 1H), 7.08 (dd, J=8.4, 1.8 Hz, 1H), 4.89 (s, 2H), 3.93 (t, J=5.7 Hz, 2H), 2.95 (t, J=5.7 Hz, 2H). LC-MS, Rt=8.132 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 8-Chloro-2-(6-chloro-1,3-benzothiazol-2-yl)-2,3,4,9-tetrahydro-1H-pyrido[3,4-b]indole (4h) prepared by method A. Yellow solid was obtained with a yield of 68%. ¹H NMR (500 MHz, Chloroform-d) δ 8.17 (s, 1H), 7.61 (d, J=2.1 Hz, 1H), 7.49 (d, J=8.6 Hz, 1H), 7.42 (d, J=8.0 Hz, 1H), 7.31-7.25 (m, 1H), 7.20 (dd, J=7.7, 1.0 Hz, 1H), 7.08 (t, J=7.8 Hz, 1H), 4.96 (s, 2H), 3.99 (t, J=5.7 Hz, 2H), 3.14-2.90 (m, 2H). LC-MS, Rt=7.006 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 2-(6-Chloro-1,3-benzothiazol-2-yl)-6-fluoro-2,3,4,9-tetrahydro-1H-pyrido[3,4-b]indole (4i) prepared by method A. Yellow solid was obtained with a yield of 48%. ¹H NMR (400 MHz, Chloroform-d) δ 7.93 (s, 1H), 7.58 (d, J=2.2 Hz, 1H), 7.44 (d, J=8.5 Hz, 1H), 7.32-7.19 (m, 3H), 7.12 (dd, J=9.3, 2.5 Hz, 1H), 6.91 (td, J=9.1, 2.5 Hz, 1H), 4.91 (s, 2H), 3.93 (t, J=5.7 Hz, 2H), 2.94 (t, J=5.7 Hz, 2H). LC-MS, Rt=7.768 min, [M+H]⁺=358.0. HRMS (ESI) m/z calcd. for C₁₈H₁₄ClFN₃S [M+H]⁺=358.0576, found 358.0535.

In another particular embodiment, the invention may include the compound: 6-Bromo-2-(6-chloro-1,3-benzothiazol-2-yl)-2,3,4,9-tetrahydro-1H-pyrido[3,4-b]indole (4j) prepared by method A. Yellow solid was obtained with a yield of 38%. ¹H NMR (400 MHz, Chloroform-d) δ 7.99 (s, 1H), 7.62-7.60 (m, 1H), 7.58 (d, J=2.2 Hz, 1H), 7.44 (d, J=8.6 Hz, 1H), 7.27-7.22 (m, 2H), 7.19 (d, J=8.6 Hz, 1H), 4.91 (s, 2H), 3.92 (t, J=5.7 Hz, 2H), 2.94 (ddd, J=5.9, 4.1, 1.7 Hz, 2H). LC-MS, Rt=8.217 min, [M+H]⁺=417.0. HRMS (ESI) m/z calcd. for C₁₈H₁₄BrClN₃S [M+H]⁺=417.9775, found 417.9819.

In another particular embodiment, the invention may include the compound: 2-(6-Chloro-1,3-benzothiazol-2-yl)-6-methoxy-2,3,4,9-tetrahydro-1H-pyrido[3,4-b]indole (4k) prepared by method A. Yellow solid was obtained with a yield of 42%. ¹H NMR (400 MHz, Chloroform-d) δ 7.79 (s, 1H), 7.57 (d, J=2.1 Hz, 1H), 7.44 (d, J=8.6 Hz, 1H), 7.24 (s, 2H), 6.93 (d, J=2.5 Hz, 1H), 6.82 (dd, J=8.8, 2.5 Hz, 1H), 4.87 (s, 2H), 3.93 (t, J=5.7 Hz, 2H), 3.84 (s, 3H), 2.95 (t, J=5.7 Hz, 2H). LC-MS, Rt=7.552 min, [M+H]⁺=370.1. HRMS (ESI) m/z calcd. for C₁₉H₁₇ClN₃OS [M+H]⁺=370.0776, found 370.0780.

In another particular embodiment, the invention may include the compound: Ethyl 6-chloro-2-(6-chloro-1,3-benzothiazol-2-yl)-2,3,4,9-tetrahydro-1H-pyrido[3,4-b]indole-1-carboxylate (4l) prepared by method A with ethyl ester modified tryptoline (6-chloro-2,3,4,9-tetrahydro-1H-Pyrido[3,4-b]indole-1-carboxylic acid ethyl ester). Pale yellow solid was obtained with a yield of 21%. ¹H NMR (400 MHz, Chloroform-d) δ 8.93 (s, 1H), 7.88 (d, J=8.7 Hz, 1H), 7.80 (d, J=2.1 Hz, 1H), 7.47 (dd, J=2.0, 1.0 Hz, 1H), 7.39 (ddd, J=8.8, 2.1, 0.9 Hz, 1H), 7.29 (d, J=8.6 Hz, 1H), 7.13 (ddd, J=8.6, 2.1, 0.9 Hz, 1H), 4.31 (qdd, J=7.1, 4.4, 0.9 Hz, 2H), 3.48 (s, 1H), 3.25 (dtt, J=23.8, 12.4, 5.6 Hz, 2H), 2.90-2.68 (m, 2H), 1.29 (td, J=7.1, 0.9 Hz, 3H). LC-MS, Rt=8.200 min, [M+H]⁺=446.0. HRMS (ESI) m/z calcd. for C₂₁HisC₁₂N₃O₂S [M+H]⁺=446.0492, found 446.0450.

In another particular embodiment, the invention may include the compound: Methyl 6-chloro-2-(6-chloro-1,3-benzothiazol-2-yl)-2,3,4,9-tetrahydro-1H-pyrido[3,4-b]indole-3-carboxylate (4m) prepared by method A with methyl ester modified tryptoline (6-chloro-2,3,4,9-tetrahydro-1H-Pyrido[3,4-b]indole-3-carboxylic acid ethyl ester). Pale yellow solid was obtained with a yield of 20%. ¹H NMR (400 MHz, Chloroform-d) δ 8.11 (d, J=8.8 Hz, 1H), 7.83 (d, J=8.6 Hz, 1H), 7.81 (d, J=2.1 Hz, 1H), 7.47 (dd, J=4.4, 2.1 Hz, 2H), 7.28 (dd, J=8.8, 2.1 Hz, 1H), 4.59 (d, J=17.1 Hz, 1H), 4.51-4.40 (m, 1H), 4.16-4.05 (m, 1H), 3.88 (d, J=4.8 Hz, 1H), 3.81 (s, 3H), 3.13 (dd, J=15.8, 4.7 Hz, 1H), 2.98-2.87 (m, 1H).LC-MS, Rt=7.860 min, [M+H]⁺=432.0. HRMS (ESI) m/z calcd. for C₂₀H₁₆C₁₂N₃O₂S [M+H]⁺=432.0335, found 432.0355.

In another particular embodiment, the invention may include the compound: Methyl 6-chloro-2-(6-chlorobenzo[d]thiazol-2-yl)-2,3,4,9-tetrahydro-1H-pyrido[3,4-b]indole-4-carboxylate (4n) prepared by method A with methyl ester modified tryptoline (6-chloro-2,3,4,9-tetrahydro-1H-Pyrido[3,4-b]indole-4-carboxylic acid methyl ester). Pale yellow solid was obtained with a yield of 82%. ¹H NMR (400 MHz, DMSO-d₆) δ 11.35 (s, 1H), 7.95 (t, J=1.7 Hz, 1H), 7.55-7.45 (m, 2H), 7.40 (d, J=8.6 Hz, 1H), 7.32 (dd, J=8.6, 2.2 Hz, 1H), 7.10 (dd, J=8.6, 2.1 Hz, 1H), 5.07 (d, J=16.6 Hz, 1H), 4.68 (dd, J=16.6, 1.5 Hz, 1H), 4.38 (dd, J=13.3, 3.1 Hz, 1H), 4.21-4.13 (m, 1H), 3.90 (dd, J=13.4, 4.5 Hz, 1H), 3.61 (s, 3H). ¹³C NMR (101 MHz, DMSO-d₆) δ 172.12, 168.74, 151.03, 134.57, 133.11, 131.96, 127.43, 127.40, 126.23, 125.18, 123.64, 121.13, 119.67, 117.81, 112.81, 109.58, 104.51, 52.00, 49.08, 45.16, 37.73. LC-MS, Rt=5.619 min, [M+H]⁺=432.0. HRMS (ESI) m/z calcd. for C₂₀H₁₆C₁₂N₃O₂S [M+H]⁺=432.0335, found 432.0355.

In another particular embodiment, the invention may include the compound: 6-Chloro-2-(6-chloro-1,3-benzothiazol-2-yl)-9-ethyl-2,3,4,9-tetrahydro-1H-pyrido[3,4-b]indole (4o) To a stirred solution of 4a (37 mg, 0.100 mmol) in dry DMF (6.0 mL), NaH (50.0 mg, 60% suspension in mineral oil, 0.300 mmol) was added portionwise under nitrogen atmosphere at 0° C. The reaction mixture was then warmed to room temperature and stirred for 30 min. After cooling to 0° C., EtI (47 mg, 0.300 mmol) was added dropwise to the reaction mixture. The reaction mixture was warmed to room temperature and stirred overnight. Water was added and the aqueous layer was extracted with ether. The combined organic layers were washed with brine, dried over anhydrous Na₂SO₄ and concentrated under reduced pressure. The residue was purified by column chromatography on silica gel (n-hexane/ethyl acetate=7/2) to give the title compound with a yield of 60%. ¹H NMR (500 MHz, Chloroform-d) δ 7.61 (d, J=2.1 Hz, 1H), 7.53-7.42 (m, 2H), 7.29 (d, J=7.9 Hz, 1H), 7.25 (d, J=8.6 Hz, 1H), 7.17 (dd, J=8.8, 2.0 Hz, 1H), 4.94 (s, 2H), 4.14 (q, J=7.3 Hz, 2H), 3.92 (t, J=5.7 Hz, 2H), 2.97 (t, J=5.8 Hz, 2H), 1.41 (t, J=7.2 Hz, 3H). ¹³C NMR (101 MHz, Chloroform-d) δ 168.88, 151.32, 134.60, 131.82, 131.59, 127.61, 126.74, 126.70, 126.67, 126.63, 125.11, 121.79, 120.60, 120.50, 119.90, 119.81, 117.91, 117.82, 107.28, 47.97, 44.91, 38.46, 21.11, 15.62. LC-MS, Rt=7.660 min, [M+H]⁺=402.1. HRMS (ESI) m/z calcd. for C₂₀H₁₈Cl₂N₃S [M+H]⁺=402.0593, found 402.0575.

In another particular embodiment, the invention may include the compound: 2-(6-Bromo-1,3-benzothiazol-2-yl)-6-chloro-2,3,4,9-tetrahydro-1H-pyrido[3,4-b]indole (4p) prepared by method A using 6-bromo-2-chloro-benzothiazole as reactant. Yellow solid was obtained with a yield of 44%. ¹H NMR (400 MHz, DMSO-d₆) δ 11.17 (s, 1H), 8.01 (d, J=1.2 Hz, 1H), 7.46 (d, J=2.0 Hz, 1H), 7.40 (d, J=1.2 Hz, 2H), 7.37 (d, J=8.6 Hz, 1H), 7.06 (dd, J=8.6, 2.1 Hz, 1H), 4.86 (s, 2H), 3.91 (t, J=5.7 Hz, 2H), 2.85 (t, J=5.7 Hz, 2H). ¹³C NMR (101 MHz, DMSO-d₆) δ 168.67, 151.55, 134.48, 132.35, 132.19, 128.93, 127.55, 123.66, 123.34, 120.87, 120.02, 117.06, 112.67, 112.63, 106.69, 47.00, 45.61, 20.29. LC-MS, Rt=7.529 min, [M+H]⁺=417.0. HRMS (ESI) m/z calcd. for C₁₈H₁₄BrClN₃S [M+H]⁺=417.9753, found 417.9729.

In another particular embodiment, the invention may include the compound: 2-(6-Chloro-1,3,4,9-tetrahydro-2H-pyrido[3,4-b]indol-2-yl)-1,3-benzothiazol-6-amine (4q) prepared by reduction of nitro precursor, which prepared by method A using 2-chloro-6-nitro-benzothiazole as reactant. Nitro precursor: yellow solid was obtained with a yield of 42%. ¹H NMR (400 MHz, DMSO-d₆) δ 11.19 (s, 1H), 8.86 (d, J=2.5 Hz, 1H), 8.63 (d, J=2.4 Hz, 1H), 7.57 (d, J=8.8 Hz, 1H), 7.49 (d, J=2.1 Hz, 1H), 7.38 (d, J=8.6 Hz, 1H), 7.07 (dd, J=8.6, 2.1 Hz, 1H), 4.97 (s, 2H), 4.04 (m, 2H), 2.97-2.86 (m, 2H).LC-MS, Rt=7.592 min, [M+H]⁺=385.0. To a solution of the nitro precursor (38.5 mg, 1.00 mmol) in AcOH (10 mL) at 0-10° C. was added Fe powder (280.0 mg, 5.00 mmol). The reaction mixture was stirred at room temperature for 24 h, was then concentrated under vacuum. The residue was diluted with H₂O and basified with saturated NaHCO₃solution. The mixture was extracted with EtOAc (2×50 mL), dried over Na₂SO₄, and concentrated. The residue was purified by flash chromatography on silica gel using ethyl acetate/hexane (2:1, v/v) as eluent. Pale yellow solid was obtained with a yield of 75%. ¹H NMR (400 MHz, Chloroform-d) δ 8.04 (s, 1H), 7.45 (d, J=2.0 Hz, 1H), 7.38 (d, J=8.5 Hz, 1H), 7.23 (d, J=8.5 Hz, 1H), 7.11 (dd, J=8.6, 2.0 Hz, 1H), 6.98 (d, J=2.4 Hz, 1H), 6.69 (dd, J=8.5, 2.4 Hz, 1H), 4.88 (s, 2H), 3.89 (t, J=5.7 Hz, 2H), 2.98-2.90 (m, 2H). LC-MS, Rt=5.273 min, [M+H]⁺=355.1. HRMS (ESI) m/z calcd. for C₁₈H₁₆ClN₄S [M+H]⁺=355.0779, found 355.0784.

In another particular embodiment, the invention may include the compound: Ethyl 2-(6-chloro-1,3,4,9-tetrahydro-2H-pyrido[3,4-b]indol-2-yl)-1,3-benzothiazole-6-carboxylate (4r) prepared by method A using 2-chloro-6-Benzothiazolecarboxylic acid ethyl ester as reactant. Yellow solid was obtained with a yield of 42%. ¹H NMR (400 MHz, DMSO-d₆) δ 11.15 (s, 1H), 8.41 (d, J=1.7 Hz, 1H), 7.85 (ddd, J=8.5, 1.9, 0.9 Hz, 1H), 7.50 (d, J=8.4 Hz, 1H), 7.46 (d, J=2.0 Hz, 1H), 7.34 (d, J=8.6 Hz, 1H), 7.03 (ddd, J=8.6, 2.2, 1.0 Hz, 1H), 4.89 (s, 2H), 4.27 (q, J=7.1 Hz, 2H), 4.01-3.90 (m, 2H), 2.86 (t, J=5.8 Hz, 2H), 1.40-1.15 (m, 3H). ¹³C NMR (101 MHz, DMSO-d₆) δ 170.83, 165.54, 156.25, 134.50, 132.05, 130.48, 127.53, 123.34, 123.01, 122.40, 120.91, 118.01, 117.09, 112.67, 106.72, 60.51, 39.94, 39.73, 39.52, 39.31, 39.10, 20.31, 14.30. LC-MS, Rt=6.810 min, [M+H]⁺=412.0. HRMS (ESI) m/z calcd. for C₂₁H₁₉ClN₃O₂S [M+H]⁺=412.0882, found 412.0869.

In another particular embodiment, the invention may include the compound: 6-Chloro-2-[6-(trifluoromethyl)-1,3-benzothiazol-2-yl]-2,3,4,9-tetrahydro-1H-pyrido[3,4-b]indole (4s) prepared by method A using 2-chloro-6-(trifluoromethyl)-benzothiazole as reactant. Pale yellow solid was obtained with a yield of 44%. ¹H NMR (400 MHz, Chloroform-d) δ 8.15 (s, 1H), 7.90-7.83 (m, 1H), 7.61-7.49 (m, 1H), 7.44 (d, J=2.0 Hz, 1H), 7.21 (dd, J=8.6, 0.6 Hz, 1H), 7.11 (dd, J=8.6, 2.0 Hz, 1H), 4.94 (t, J=1.6 Hz, 2H), 3.96 (t, J=5.7 Hz, 2H), 2.95 (tt, J=5.6, 1.5 Hz, 2H). LC-MS, Rt=8.021 min, [M+H]⁺=408.1. HRMS (ESI) m/z calcd. for C₁H₁₄ClF₃N₃S [M+H]⁺=408.0544, found 408.0568.

In another particular embodiment, the invention may include the compound: 6-Chloro-2-[6-(trifluoromethoxy)-1,3-benzothiazol-2-yl]-2,3,4,9-tetrahydro-1H-pyrido[3,4-b]indole (4t) prepared by method A using 2-chloro-6-(trifluoromethoxy)-benzothiazole as reactant. Pale yellow solid was obtained with a yield of 84%. ¹H NMR (400 MHz, Chloroform-d) δ 8.06 (s, 1H), 7.54-7.46 (m, 2H), 7.44 (d, J=2.0 Hz, 1H), 7.23 (d, J=10.8 Hz, 1H), 7.20-7.12 (m, 1H), 7.11 (dd, J=8.6, 2.1 Hz, 1H), 4.91 (s, 2H), 3.93 (t, J=5.7 Hz, 2H), 2.94 (t, J=5.7 Hz, 2H). LC-MS, Rt=7.031 min, [M+H]⁺=424.0. HRMS (ESI) m/z calcd. for C₁₉H₁₄ClF₃N₃OS [M+H]⁺=424.0493, found 424.0534.

In another particular embodiment, the invention may include the compound: 6-Chloro-2-(7-chloro-1,3-benzothiazol-2-yl)-2,3,4,9-tetrahydro-1H-pyrido[3,4-b]indole (4u) prepared by method A using 2,7-dichloro-benzothiazole as reactant. Pale yellow solid was obtained with a yield of 42%. ¹H NMR (400 MHz, DMSO-d₆) δ 11.10 (s, 1H), 7.38 (d, J=2.0 Hz, 1H), 7.36 (d, J=8.0 Hz, 1H), 7.29 (d, J=8.6 Hz, 1H), 7.22 (t, J=8.0 Hz, 1H), 7.10-7.02 (m, 1H), 6.98 (dd, J=8.5, 2.1 Hz, 1H), 4.80 (s, 2H), 3.85 (t, J=5.7 Hz, 2H), 2.79 (t, J=5.7 Hz, 2H). ¹³C NMR (101 MHz, DMSO-d₆) δ 167.72, 153.31, 134.51, 132.08, 129.85, 127.54, 127.49, 124.81, 123.37, 120.89, 117.23, 117.11, 117.03, 112.67, 106.69, 47.16, 45.61, 20.30. LC-MS, Rt=8.288 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 6-Chloro-2-(5-chloro-1,3-benzothiazol-2-yl)-2,3,4,9-tetrahydro-1H-pyrido[3,4-b]indole (4v) prepared by method A using 2,5-dichloro-benzothiazole as reactant. Pale yellow solid was obtained with a yield of 44%. ¹H NMR (400 MHz, DMSO-d₆) δ 11.12 (s, 1H), 7.74 (d, J=8.4 Hz, 1H), 7.44 (dd, J=17.0, 2.0 Hz, 2H), 7.31 (d, J=8.6 Hz, 1H), 7.04 (dd, J=8.4, 2.1 Hz, 1H), 7.00 (dd, J=8.6, 2.1 Hz, 1H), 4.82 (s, 2H), 3.87 (t, J=5.8 Hz, 2H), 2.81 (t, J=5.7 Hz, 2H). ¹³C NMR (101 MHz, DMSO-d₆) δ 169.67, 153.59, 134.50, 132.17, 130.75, 129.02, 127.56, 123.36, 122.59, 122.53, 121.04, 120.90, 118.06, 117.99, 117.12, 117.04, 112.68, 109.60, 106.71, 47.02, 45.62, 20.29. LC-MS, Rt=8.002 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 6-Chloro-2-(4-chloro-1,3-benzothiazol-2-yl)-2,3,4,9-tetrahydro-1H-pyrido[3,4-b]indole (4w) prepared by method A using 2,4-dichloro-benzothiazole as reactant. Pale yellow solid was obtained with a yield of 38%. ¹H NMR (400 MHz, Chloroform-d) δ 7.97 (s, 1H), 7.50 (dd, J=7.9, 1.1 Hz, 1H), 7.41 (d, J=1.9 Hz, 1H), 7.31 (dd, J=7.9, 1.1 Hz, 1H), 7.21 (dd, J=8.6, 0.6 Hz, 1H), 7.10 (dd, J=8.6, 2.0 Hz, 1H), 7.00 (t, J=7.9 Hz, 1H), 4.95 (s, 2H), 3.92 (t, J=5.7 Hz, 1H), 2.91 (t, J=5.7 Hz, 2H). ¹³C NMR (75 MHz, CDCl₃) δ 168.88, 149.65, 134.66, 131.85, 131.09, 127.96, 126.55, 125.58, 123.57, 122.34, 122.13, 119.47, 117.75, 112.05, 108.38, 47.97, 45.51, 20.95. LC-MS, Rt=7.969 min, [M+H]⁺=374.0. HRMS (ESI) m/z calcd. for C₁₈H₁₄Cl₂N₃S [M+H]⁺=374.0280, found 374.0262.

In another particular embodiment, the invention may include the compound: 6-Chloro-2-(4,6-dichloro-1,3-benzothiazol-2-yl)-2,3,4,9-tetrahydro-1H-pyrido[3,4-b]indole (4x) prepared by method A using 2,4,6-trichloro-benzothiazole as reactant. Pale yellow solid was obtained with a yield of 48%. ¹H NMR (500 MHz, Chloroform-d) δ 8.10 (s, 1H), 7.48 (s, 1H), 7.41 (s, 1H), 7.33 (s, 1H), 7.22 (d, J=8.6 Hz, 1H), 7.13 (d, J=8.7 Hz, 1H), 4.90 (s, 2H), 3.89 (d, J=5.9 Hz, 2H), 2.90 (t, J=5.9 Hz, 2H). LC-MS, Rt=7.425 min, [M+H]⁺=408.0. HRMS (ESI) m/z calcd. for C₁₈H₁₃Cl₃N₃S [M+H]⁺=407.9891, found 407.9863.

In another particular embodiment, the invention may include the compound: 6-Chloro-2-(4,5,6-trichloro-1,3-benzothiazol-2-yl)-2,3,4,9-tetrahydro-1H-pyrido[3,4-b]indole (4y) prepared by method B using 1,2,3-trichloro-4-isothiocyanato-benzene as reactant. Pale yellow solid was obtained with a yield of 70%. ¹H NMR (500 MHz, Chloroform-d) δ 8.00 (s, 1H), 7.62 (s, 1H), 7.46 (d, J=2.0 Hz, 1H), 7.30-7.24 (m, 1H), 7.15 (dd, J=8.6, 2.0 Hz, 1H), 4.99 (s, 2H), 3.95 (t, J=5.7 Hz, 2H), 2.97 (t, J=5.7 Hz, 2H). LC-MS, Rt=7.711 min, [M+H]⁺=443.9. HRMS (ESI) m/z calcd. for C₁₈H₁₂Cl₄N₃S [M+H]⁺=441.9501, found 441.9526.

In another particular embodiment, the invention may include the compound: 6-Chloro-2-(4-chloro-6-methoxy-1,3-benzothiazol-2-yl)-2,3,4,9-tetrahydro-1H-pyrido[3,4-b]indole (4z) prepared by method B using 2-chloro-1-isothiocyanato-4-methoxy-benzene as reactant. Pale yellow solid was obtained with a yield of 89%. ¹H NMR (400 MHz, Chloroform-d) δ 7.96 (s, 1H), 7.42 (d, J=2.1 Hz, 1H), 7.22 (d, J=8.6 Hz, 1H), 7.10 (dd, J=8.6, 2.0 Hz, 1H), 7.06 (d, J=2.4 Hz, 1H), 6.94 (d, J=2.4 Hz, 1H), 4.92 (s, 2H), 3.89 (t, J=5.7 Hz, 21H), 3.80 (s, 3H), 2.91 (t, J=5.7 Hz, 2H). LC-MS, Rt=8.702 min, [M+H]⁺=404.0. HRMS (ESI) m/z calcd. for C₁₉H₁₆Cl₂N₃OS [M+H]⁺=404.0386, found 404.0377.

In another particular embodiment, the invention may include the compound: 4-Chloro-2-(6-chloro-1,3,4,9-tetrahydro-2H-pyrido[3,4-b]indol-2-yl)-1,3-benzothiazol-6-ol (4aa) prepared by using BBr₃ for the de-protection of 4z according to the literature. A solution of BBr₃ (3.0 mL, 3.00 mmol, 1M in CH₂Cl₂) was added to a solution of compound 4z (201.0 mg, 0.5 mmol) in anhydrous CH₂Cl₂ (4.0 mL) under N₂ at −78° C. and stirred at this temperature for 1 h before warming up to room temperature and stirred for another 12 h. The reaction mixture was quenched with methanol. The solvent was removed under vacuum and the residue was extracted with CH₂Cl₂ after basified with saturated NaHCO₃solution. The combined organic layer was washed with brine, dried over Na₂SO₄ and concentrated under vacuum. The residue was purified by silica gel column chromatography to give the product with yield of 90%. ¹H NMR (400 MHz, Chloroform-d) δ 7.93 (s, 1H), 7.43 (d, J=2.0 Hz, 1H), 7.23 (d, J=6.6 Hz, 1H), 7.11 (dd, J=8.6, 2.0 Hz, 1H), 7.03 (dd, J=20.2, 2.4 Hz, 1H), 6.91 (dd, J=27.1, 2.4 Hz, 1H), 4.93 (d, J=2.7 Hz, 2H), 3.89 (td, J=5.7, 2.4 Hz, 2H), 3.80 (s, 1H), 2.92 (t, J=5.8 Hz, 2H). LC-MS, Rt=7.926 min, [M+H]⁺=390.0. HRMS (ESI) m/z calcd. for C₁₈H₁₄Cl₂N₃OS [M+H]⁺=390.0230, found 390.0198.

In another particular embodiment, the invention may include the compound: 2-((4-Chloro-2-(6-chloro-1,3,4,9-tetrahydro-2H-pyrido[3,4-b]indol-2-yl)benzo[d]thiazol-6-yl)oxy)ethan-1-amine (4ab). A suspension of 4-chloro-2-(6-chloro-1,3,4,9-tetrahydro-2H-pyrido[3,4-b]indol-2-yl)-1,3-benzothiazol-6-ol 4aa (82.5 mg, 0.200 mmol), N-(2-hydroxyethyl)phthalimide (57.5 mg, 0.300 mmol) and triphenylphosphine (78.5 mg, 0.300 mmol) in anhydrous THF (5 mL) was stirred under an argon atmosphere until a clear solution was obtained. Then, the solution of diisopropyl azodicarboxylate (DIAD) in THF (61 mg, 0.300 mmol) were added dropwise and the reaction mixture was refluxed for 15 h. The solvents were evaporated under reduced pressure and the residue was purified by silica gel column chromatography. To a solution of the product obtained from the previous step in absolute ethanol (5 mL) were added 0.2 mL of hydrazine hydrate and 0.25 mL of acetic acid. The reaction mixture was refluxed for 3 h. After cooling down to room temperature, the precipitates were removed by filtration and the filtrate was concentrated under reduced pressure. The residue was resuspended in dichloromethane (50 mL) and extracted with 20% NaOH solution (3×50 mL). The organic layers were dried over Na₂SO₄, concentrated under reduced pressure. The residue was purified by silica gel column chromatography with a yield of 50%. ¹H NMR (500 MHz, Chloroform-d) δ 8.31 (s, 1H), 7.46 (d, J=2.0 Hz, 1H), 7.14 (dd, J=8.5, 2.0 Hz, 1H), 7.10 (d, J=2.4 Hz, 1H), 7.03-6.95 (m, 2H), 4.97 (s, 2H), 4.00 (t, J=5.1 Hz, 2H), 3.93 (t, J=5.7 Hz, 2H), 3.57 (m, 2H), 2.96 (t, J=5.7 Hz, 2H), 2.91 (s, 2H). LC-MS, Rt=6.453 min, [M+H]⁺=433.0. HRMS (ESI) m/z calcd. for C₂₀H₁₉Cl₂N₄OS [M+H]⁺=433.0652, found 433.0703.

In another particular embodiment, the invention may include the compound: 2-(6-Bromo-4-chloro-1,3-benzothiazol-2-yl)-6-chloro-2,3,4,9-tetrahydro-1H-pyrido[3,4-b]indole (4ac) prepared by method B using 4-bromo-2-chloro-1-isothiocyanato-benzene as reactant. Pale yellow solid was obtained with a yield of 95%. ¹H NMR (400 MHz, Acetone-d₆) δ 7.89 (dd, J=1.9, 0.6 Hz, 0H), 7.48 (dd, J=6.7, 2.0 Hz, 1H), 7.39 (d, J=8.6 Hz, 0H), 7.07 (dd, J=8.6, 2.1 Hz, 0H), 4.99 (d, J=1.6 Hz, 1H), 4.03 (t, J=6.7 Hz, 2H), 2.98 (ddd, J=7.3, 3.7, 1.7 Hz, 1H). LC-MS, Rt=7.565 min, [M+H]⁺=453.9. HRMS (ESI) m/z calcd. for C₁₈H₁₃BrCl₂N₃S [M+H]⁺=451.9386, found 451.9359.

In another particular embodiment, the invention may include the compound: 4-Chloro-2-(6-chloro-1,3,4,9-tetrahydro-2H-pyrido[3,4-b]indol-2-yl)-1,3-benzothiazol-6-amine (4ad). 10 mL reaction vessel was charged with Cu₂O (14.3 mg, 0.100 mmol), 2-[6-bromo-4-(trifluoromethyl)-1,3-benzothiazol-2-yl]-6-chloro-2,3,4,9-tetrahydro-1H-pyrido[3,4-b]indole 4ac (906.0 mg, 2.00 mmol), 1.5 mL of N-methyl pyrrolidinone (NMP), 1.5 mL of ammonium hydroxide solution (28% NH₃, 20.0 mmol) and a magnetic stir bar. The vessel was sealed with a Teflon screw cap, and was stirred at 80° C. for 48 h. The reaction mixture was cooled to room temperature, quenched with water, extracted with diethyl ether and dried over Na₂SO₄. The solvents were removed under vacuum and the residue was purified by silica gel flash chromatography. Red solid was obtained as product with a yield of 56%. ¹H NMR (400 MHz, DMSO-d₆) δ 11.08 (s, 1H), 7.38 (d, J=2.1 Hz, 1H), 7.27 (d, J=8.6 Hz, 1H), 6.97 (dd, J=8.6, 2.1 Hz, 1H), 6.81 (d, J=2.1 Hz, 1H), 6.59 (d, J=2.1 Hz, 1H), 5.08 (s, 2H), 4.74 (s, 2H), 3.75 (t, J=5.7 Hz, 2H), 2.76 (t, J=5.7 Hz, 2H). ¹³C NMR (101 MHz, DMSO-d₆) δ 165.12, 144.93, 140.05, 134.68, 133.04, 132.82, 127.87, 123.56, 122.53, 121.07, 117.29, 113.33, 112.85, 106.93, 104.55, 47.30, 45.70, 20.54. LC-MS, Rt=8.066 min, [M+H]⁺=389.0. HRMS (ESI) m/z calcd. for C₁₈H₁₅Cl₂N₄S [M+H]⁺=389.0390, found 389.0378.

In another particular embodiment, the invention may include the compound: 2-amino-N-(4-chloro-2-(6-chloro-1,3,4,9-tetrahydro-2H-pyrido[3,4-b]indol-2-yl)benzo[d]thiazol-6-yl)acetamide hydrochloride (4ae) To a round bottom flask was added 4-chloro-2-(6-chloro-1,3,4,9-tetrahydro-2H-pyrido[3,4-b]indol-2-yl)-1,3-benzothiazol-6-amine 4ad (21.0 mg, 0.050 mmol), Boc-Gly-OH (10.5 mg, 0.06 mmol), DMAP (7.5 mg, 0.060 mmol), EDCI (9.5 mg, 0.060 mmol) and DCM (2 mL), and the reaction mixture was stirred for 2 h. The solvents were removed under vacuum and the residue was purified by silica gel flash chromatography using ethyl acetate/hexane as eluent. To a round bottom flask was added the obtained product (35 mg, 0.050 mmol) and HCl in dioxane (2.0 mL, 4 M), and the reaction mixture was stirred for 4 h. The solvents were removed under vacuum and red solid was obtained as product with a yield of 90%. ¹H NMR (500 MHz, Methanol-d₄) δ 8.11-7.99 (m, 1H), 7.62 (dd, J=17.5, 2.0 Hz, 1H), 7.43 (dd, J=3.6, 2.0 Hz, 1H), 7.30 (dd, J=8.6, 1.7 Hz, 1H), 7.06 (dt, J=8.6, 2.4 Hz, 1H), 4.99 (s, 2H), 4.09 (t, J=5.8 Hz, 2H), 3.88 (s, 2H), 2.99 (t, J=6.1 Hz, 2H). LC-MS, Rt=6.636 min, [M+H]⁺=446.0. HRMS (ESI) m/z calcd. for C₂₀H₁₈Cl₂N₅OS [M+H]⁺=446.0604, found 446.0634.

In another particular embodiment, the invention may include the compound: 6-Chloro-2-[4-(trifluoromethyl)-1,3-benzothiazol-2-yl]-2,3,4,9-tetrahydro-1H-pyrido[3,4-b]indole (4af) prepared by method A using 2-chloro-4-(trifluoromethyl)-benzothiazole as reactant. Pale yellow solid was obtained with a yield of 41%. ¹H NMR (400 MHz, Chloroform-d) δ 7.96 (s, 1H), 7.76 (dd, J=7.9, 1.2 Hz, 1H), 7.59-7.52 (m, 1H), 7.40 (d, J=2.0 Hz, 1H), 7.20 (dd, J=8.5, 0.6 Hz, 1H), 7.14-7.07 (m, 2H), 4.95 (s, 2H), 3.91 (t, J=5.7 Hz, 2H), 2.88 (t, J=5.7 Hz, 2H). LC-MS, Rt=7.142 min, [M+H]⁺=408.0. HRMS (ESI) m/z calcd. for C₁₉H₁₄ClF₃N₃S [M+H]⁺=408.0544, found 408.0568.

In another particular embodiment, the invention may include the compound: 2-[6-Bromo-4-(trifluoromethyl)-1,3-benzothiazol-2-yl]-6-chloro-2,3,4,9-tetrahydro-1H-pyrido[3,4-b]indole (4ag) prepared by method B using 4-bromo-1-isothiocyanato-2-(trifluoromethyl)-benzene as reactant. Pale yellow solid was obtained with a yield of 80%. ¹H NMR (400 MHz, Acetone-d₆) δ 8.21 (s, 1H), 7.69 (s, 1H), 7.47 (s, 1H), 7.39 (dd, J=8.6, 1.6 Hz, 1H), 7.08 (d, J=8.6 Hz, 1H), 5.02 (s, 2H), 4.06 (t, J=5.2 Hz, 2H), 2.99 (tt, J=3.9, 1.9 Hz, 2H). LC-MS, Rt=7.644 min, [M+H]⁺=487.9. HRMS (ESI) m/z calcd. for C₁₉H₁₃BrClF₃N₃S [M+H]⁺=485.9649, found 485.9641.

In another particular embodiment, the invention may include the compound: 2-(6-Chloro-1,3,4,9-tetrahydro-2H-pyrido[3,4-b]indol-2-yl)-4-(trifluoromethyl)-1,3-benzothiazol-6-aminium chloride (4ah). 10 mL reaction vessel was charged with Cu₂O (14.3 mg, 0.100 mmol), 2-[6-bromo-4-(trifluoromethyl)-1,3-benzothiazol-2-yl]-6-chloro-2,3,4,9-tetrahydro-1H-pyrido[3,4-b]indole 4ag (973.4 mg, 2.00 mmol), 1.5 mL of N-methyl pyrrolidinone (NMP), 1.5 mL of ammonium hydroxide solution (28% NH₃, 20.0 mmol) and a magnetic stir bar. The vessel was sealed with a Teflon screw cap, and was stirred at 80° C. for 48 h. Then the reaction mixture was cooled to 25° C., quenched with water, extracted with diethyl ether and dried over anhydrous Na₂SO₄. The solvents were removed under vacuum and the residue was purified by silica gel flash chromatography. Then, to a round bottom flask was added the product obtained from above (50 mg, 0.01 mmol) and HCl in 1,4-dioxane (1 mL, 4 M), and the reaction mixture was stirred for 1 h. The solvents were removed under vacuum and red solid was obtained as product with a yield of 78%. ¹H NMR (400 MHz, Chloroform-d) δ 7.94 (s, 1H), 7.39 (d, J=2.0 Hz, 1H), 7.18 (d, J=8.6 Hz, 1H), 7.13-7.05 (m, 2H), 6.93 (d, J=2.3 Hz, 1H), 4.88 (s, 2H), 3.85 (t, J=5.7 Hz, 2H), 2.86 (t, J=5.8 Hz, 2H).). LC-MS, Rt=8.229 min, [M−Cl]+=423.0. HRMS (ESI) m/z calcd. for C₁₉H₁₅ClF₃N₄S [M−Cl]+=423.0653, found 423.0564.

In another particular embodiment, the invention may include the compound: 2-(6-Chloro-1,3,4,9-tetrahydro-2H-pyrido[3,4-b]indol-2-yl)-N-methyl-4-(trifluoromethyl)-1,3-benzothiazol-6-amine (4ai). Sodium metal (12.5 mg, 0.500 mmol) was added portionwise to anhydrous MeOH (2 mL) at 0° C. After complete consumption of the sodium metal, 2-(6-chloro-1,3,4,9-tetrahydro-2H-pyrido[3,4-b]indol-2-yl)-4-(trifluoromethyl)-1,3-benzothiazol-6-aminium chloride 4ah (25.0 mg, 0.500 mmol) and paraformaldehyde (15 mg, 0.500 mmol) were added at room temperature, and the mixture was stirred for 2 h at reflux to give an imine intermediate. The mixture was then reacted with NaBH₄ (0.500 mmol) at 0° C., and then refluxed for an additional 2 h. The reaction mixture was then cooled to room temperature, and the solvent was removed under reduced pressure. The residue was diluted with CH₂Cl₂ (5 mL), washed with water (3 mL), dried over Na₂SO₄ and concentrated to give a residue that was purified via silica gel column chromatography with a yield of 77%. ¹H NMR (400 MHz, Chloroform-d) δ 7.93 (s, 1H), 7.40 (d, J=2.0 Hz, 1H), 7.19 (d, J=8.5 Hz, 1H), 7.09 (dd, J=8.6, 2.0 Hz, 1H), 6.99 (d, J=2.4 Hz, 1H), 6.86 (d, J=2.4 Hz, 1H), 4.90 (s, 2H), 3.86 (t, J=5.7 Hz, 2H), 3.14-2.62 (m, 5H). LC-MS, Rt=8.081 min, [M+H]⁺=437.1. HRMS (ESI) m/z calcd. for C₂₀H₁₇ClF₃N₄S [M+H]⁺=437.0810, found 437.0849.

In another particular embodiment, the invention may include the compound: N₁-(2-(6-chloro-1,3,4,9-tetrahydro-2H-pyrido[3,4-b]indol-2-yl)-4-(trifluoromethyl)benzo[d]thiazol-6-yl)ethane-1,2-diamine hydrochloride (4aj). To a solution of 2-(6-Chloro-1,3,4,9-tetrahydro-2H-pyrido[3,4-b]indol-2-yl)-4-(trifluoromethyl)-1,3-benzothiazol-6-aminium chloride 4ah (49.0 mg, 0.100 mmol, 1 equiv) in dichloroethane at room temperature were added N-Boc-2-aminoacetaldehyde (19.0 mg, 0.120 mmol), AcOH (6.0 mg, 0.100 mmol) and NaHB(OAc)₃ (42.4 mg, 0.200 mmol). The resulting mixture was stirred for 16 h. The solvent was then removed in vacuum. The residue was redissolved in ethyl acetate, washed with saturated NaHCO₃solution, dried over Na₂SO₄ and concentrated in vacuo. The intermediate was obtained after purification by silica gel flash chromatography with a yield of 60%. To a round bottom flask was added the obtained product from above (25 mg, 0.050 mmol), and HCl in 1,4-dioxane (2 mL, 4 M). The reaction mixture was stirred at room temperature for 4 h. The solvents were then removed under vacuum and red solid was obtained as product with a yield of 90%. ¹H NMR (500 MHz, Methanol-d₄) δ 7.42 (d, J=2.1 Hz, 1H), 7.30 (dd, J=8.5, 6.3 Hz, 2H), 7.14-6.89 (m, 2H), 3.99 (s, 2H), 3.75 (dd, J=6.1, 4.8 Hz, 1H), 3.64-3.57 (m, 1H), 3.47 (d, J=6.6 Hz, 2H), 3.19 (t, J=6.0 Hz, 2H), 2.94 (t, J=5.8 Hz, 2H). LC-MS, Rt=6.706 min, [M−Cl]+=466.0. HRMS (ESI) m/z calcd. for C₂₁H₂₀ClF₃N₅S [M+H]⁺=466.1075, found 466.1111.

In another particular embodiment, the invention may include the compound: 6-Chloro-2-(6-chlorobenzo[d]thiazol-2-yl)-2,3,4,9-tetrahydro-1H-pyrido[3,4-b]indole-4-carboxylic acid (8) The mixture of methyl 6-chloro-2-(6-chlorobenzo[d]thiazol-2-yl)-2,3,4,9-tetrahydro-1H-pyrido[3,4-b]indole-4-carboxylate 4n (20 mg, 0.046 mmol) and lithium hydroxide (20 mg, 0.46 mmol) were dissolved in H₂O and MeOH (1 mL, v:v=1:1), and stirred at room temperature overnight. The reaction was then quenched with 1 N HCl to adjust the pH to around 5, extracted with ethyl estate. The combined organic phase dried over Na₂SO₄, concentrated under reduced pressure to afford the desired acid product (20 mg, 99%). ¹H NMR (500 MHz, DMSO-d₆) δ 12.67 (s, 1H), 11.32 (s, 1H), 7.96 (d, J=2.2 Hz, 1H), 7.53 (d, J=2.1 Hz, 1H), 7.49 (d, J=8.6 Hz, 1H), 7.40 (d, J=8.6 Hz, 1H), 7.32 (dd, J=8.6, 2.3 Hz, 1H), 7.09 (dd, J=8.6, 2.1 Hz, 1H), 5.05 (d, J=16.5 Hz, 1H), 4.74-4.63 (m, 1H), 4.34 (d, J=13.2 Hz, 1H), 4.06 (s, 1H), 3.87 (dd, J=13.2, 4.6 Hz, 1H). LC-MS, Rt=8.571 min, [M+H]⁺=418.0. HRMS (ESI) m/z calcd. for C₁₉H₁₄Cl₂N₃O₂S [M+H]⁺=418.0179, found 418.0175.

In another particular embodiment, the invention may include the compound: 6-Chloro-2-(6-chlorobenzo[d]thiazol-2-yl)-N,N-dimethyl-2,3,4,9-tetrahydro-1H-pyrido[3,4-b]indole-4-carboxamide (9) To the mixture of 6-chloro-2-(6-chlorobenzo[d]thiazol-2-yl)-2,3,4,9-tetrahydro-1H-pyrido[3,4-b]indole-4-carboxylic acid 8 (21 mg, 0.05 mmol) in anhydrous DCM (1 mL) was added oxalyl chloride (8.6 μL, 0.1 mmol) and 3 drops DMF. After the mixture was stirred at room temperature for 2 h, dimethylamine (26 μL, 0.25 mmol) was added, and the mixture was stirred at room temperature for another 2 h. The reaction mixture was diluted with DCM, washed with water and brine, dried over Na₂SO₄ and concentrated. The residue was purified by silica gel flash chromatography (hexane/EA=1:1 to 1:2) to afford the title compound 9 (19 mg, 85%). ¹H NMR (500 MHz, Chloroform-d) δ 9.21 (s, 1H), 7.47 (d, J=2.1 Hz, 1H), 7.39 (d, J=8.6 Hz, 1H), 7.20 (dd, J=8.6, 2.2 Hz, 1H), 7.12 (d, J=1.9 Hz, 1H), 6.91 (dd, J=8.6, 2.0 Hz, 1H), 6.75 (d, J=8.5 Hz, 1H), 4.60 (d, J=16.1 Hz, 1H), 4.41 (dd, J=7.7, 4.9 Hz, 1H), 4.28-4.14 (m, 2H), 3.92 (dd, J=13.3, 7.8 Hz, 1H), 3.40 (s, 3H), 3.15 (s, 3H). LC-MS, Rt=5.222 min, [M+H]⁺=445.0. HRMS (ESI) m/z calcd. for C₂₁H₁₉Cl₂N₄OS [M+H]⁺=445.0646, found 445.0685.

In another particular embodiment, the invention may include the compound: (6-Chloro-2-(6-chlorobenzo[d]thiazol-2-yl)-2,3,4,9-tetrahydro-1H-pyrido[3,4-b]indol-4-yl)methanol (10) To the solution of methyl 6-chloro-2-(6-chlorobenzo[d]thiazol-2-yl)-2,3,4,9-tetrahydro-1H-pyrido[3,4-b]indole-4-carboxylate 4n (43.2 mg, 0.1 mmol) in anhydrous THF (1 mL) was added LiAlH₄ (11.4 mg, 0.3 mmol) in three portions under Argon. The reaction mixture was stirred at room temperature overnight. Then quenched with 2 N NaOH solution and water. The precipitate was filtered, and the solid was washed with MeOH three times. The combined filtrate were concentrated under reduced pressure. The residue was purified by silica gel flash chromatography to afford the alcohol 10 (39.3 mg, 97%). ¹H NMR (500 MHz, Chloroform-d) δ 8.50 (s, 1H), 7.52 (d, J=2.2 Hz, 1H), 7.50 (d, J=2.0 Hz, 1H), 7.40 (d, J=8.6 Hz, 1H), 7.25-7.17 (m, 2H), 7.11 (dd, J=8.6, 2.0 Hz, 1H), 4.97 (d, J=16.1 Hz, 1H), 4.67 (dd, J=16.0, 1.6 Hz, 1H), 4.60-4.47 (m, 1H), 3.97 (dd, J=11.3, 4.9 Hz, 1H), 3.62 (dd, J=13.5, 3.7 Hz, 1H), 3.57 (dd, J=11.3, 9.6 Hz, 1H), 3.35-3.25 (m, 1H). ¹³C NMR (101 MHz, Chloroform-d) δ 169.55, 150.73, 134.70, 131.55, 131.13, 127.60, 126.98, 126.84, 125.87, 122.62, 120.62, 119.67, 117.92, 112.28, 109.28, 63.31, 48.39, 46.55, 35.90. LC-MS, Rt=9.347 min, [M+H]⁺=404.1. HRMS (ESI) m/z calcd. for C₁₉H₁₆Cl₂N₃OS [M+H]⁺=404.0386, found 404.0377.

In another particular embodiment, the invention may include the compound: 6-Chloro-2-(6-chloro-4-((methoxymethoxy)methyl)-1,3,4,9-tetrahydro-2H-pyrido[3,4-b]indol-2-yl)benzo[d]thiazole (11) To the mixture of (6-chloro-2-(6-chlorobenzo[d]thiazol-2-yl)-2,3,4,9-tetrahydro-1H-pyrido[3,4-b]indol-4-yl)methanol 10 (15 mg, 0.0371 mmol) and N,N-Diisopropylethylamine (20 μL, 0.112 mmol) in anhydrous DCM (1 mL) was added MOM chloride (10 μL, 0.132 mmol). The reaction mixture was stirred at room temperature for 2 h. The reaction mixture was then concentrated and purified by silica gel flash chromatography to afford the title product 11 (5.0 mg, 30%). ¹H NMR (500 MHz, Chloroform-d) δ 8.13 (s, 1H), 7.59 (d, J=2.1 Hz, 1H), 7.54 (d, J=2.0 Hz, 1H), 7.45 (d, J=8.6 Hz, 1H), 7.27-7.22 (m, 2H), 7.14 (dd, J=8.6, 2.0 Hz, 1H), 5.24 (d, J=16.3 Hz, 1H), 4.77-4.67 (m, 2H), 4.65 (dd, J=16.3, 1.5 Hz, 1H), 4.23 (dd, J=13.2, 2.6 Hz, 1H), 3.86 (dd, J=9.9, 4.3 Hz, 1H), 3.73 (dd, J=13.2, 3.9 Hz, 1H), 3.55 (t, J=9.9 Hz, 1H), 3.41 (s, 3H). LC-MS, Rt=8.249 min, [M+H]⁺=448.0. HRMS (ESI) m/z calcd. for C₂₁H₂₀Cl₂N₃O₂S [M+H]⁺=448.0643, found 448.0659.

In another particular embodiment, the invention may include the compound: 2-(((6-Chloro-2-(6-chlorobenzo[d]thiazol-2-yl)-2,3,4,9-tetrahydro-1H-pyrido[3,4-b]indol-4-yl)methyl)thio)ethan-1-amine hydrochloride (12a) To the solution of triphenylphosphine (58 mg, 0.22 mmol) in anhydrous DCM (2 mL) under Argon, was added iodine (28 mg, 0.22 mmol). After the mixture was stirred for 10 min, imidazole (26 mg, 0.37 mmol) was added into the mixture and stirred for another 10 min. (6-chloro-2-(6-chlorobenzo[d]thiazol-2-yl)-2,3,4,9-tetrahydro-1H-pyrido[3,4-b]indol-4-yl)methanol 10 (41 mg, 0.1 mmol) in anhydrous DCM (1 mL) was added slowly and stirred at room temperature for 1 h. The reaction mixture was concentrated and purified by silica gel flash chromatography to afford the iodinated intermediate (36 mg, 70%). The mixture of iodinated intermediate (30 mg, 0.058 mmol) and tert-butyl (2-mercaptoethyl)carbamate (0.012 mL, 5.0 equiv) and sodium hydroxide (2.3 mg, 0.058 mmol) in tert-Butyl alcohol (1 mL) was heated to 120° C. for 4 h. The reaction mixture was concentrated and purified by silica gel flash chromatography to afford the N-Boc-12a (26 mg, 78%). ¹H NMR (500 MHz, Chloroform-d) δ 8.74 (s, 1H), 7.56 (d, J=2.2 Hz, 1H), 7.51 (s, 1H), 7.42 (d, J=8.7 Hz, 1H), 7.22 (dd, J=8.6, 2.2 Hz, 1H), 7.18 (d, J=8.6 Hz, 1H), 7.09 (dd, J=8.6, 2.0 Hz, 1H), 5.18 (d, J=16.2 Hz, 1H), 5.05 (s, 1H), 4.59-4.51 (m, 1H), 4.33 (dd, J=13.2, 2.3 Hz, 1H), 3.66 (dd, J=13.3, 3.8 Hz, 1H), 3.34 (dh, J=26.6, 6.5 Hz, 2H), 3.20 (d, J=11.0 Hz, 1H), 3.00 (dd, J=13.3, 3.5 Hz, 1H), 2.75 (t, J=6.8 Hz, 2H), 2.58 (dd, J=13.2, 10.9 Hz, 1H), 1.45 (s, 9H). ³C NMR (101 MHz, Chloroform-d) δ 169.44, 155.99, 151.02, 134.76, 131.81, 131.11, 127.10, 126.93, 126.72, 125.75, 122.52, 120.66, 119.79, 117.65, 112.37, 110.87, 79.78, 77.36, 50.78, 45.47, 39.98, 35.07, 33.32, 33.24, 28.54. LC-MS, Rt=9.058 min, [M+H]⁺=563.1. HRMS (ESI) m/z calcd. for C₂₆H₂₉Cl₂N₄O₂S₂ [M+H]⁺=563.1104, found 563.1185. N-Boc protected 12a was dissolved in HCl in 1,4-dioxane solution (1 mL, 4 M) and stirred at room temperature overnight. The reaction mixture was concentrated to afford the title compound 12a in quantitative yield (18 mg, 99%). ¹H NMR (500 MHz, DMSO-d₆) δ 11.50 (s, 1H), 8.15 (d, J=6.2 Hz, 2H), 7.98 (d, J=2.2 Hz, 1H), 7.63 (d, J=2.1 Hz, 1H), 7.51 (d, J=8.6 Hz, 1H), 7.39 (d, J=8.6 Hz, 1H), 7.33 (dd, J=8.6, 2.3 Hz, 1H), 7.08 (dd, J=8.6, 2.1 Hz, 1H), 5.10 (d, J=16.5 Hz, 1H), 4.75 (d, J=16.5 Hz, 1H), 4.26 (d, J=13.3 Hz, 1H), 3.81 (dd, J=13.3, 3.9 Hz, 1H), 3.76-3.62 (m, 1H), 3.53-3.41 (m, 1H), 3.37-3.29 (m, 1H), 3.11-2.97 (m, 3H), 2.95 (q, J=6.7 Hz, 1H), 2.86 (ddd, J=13.9, 8.0, 6.3 Hz, 1H), 2.59 (dd, J=13.4, 10.3 Hz, 1H). ¹³C NMR (101 MHz, DMSO-d₆) δ 169.12, 149.33, 134.66, 132.12, 130.84, 126.83, 126.60, 125.60, 123.56, 121.35, 121.09, 119.07, 117.52, 112.88, 109.21, 60.22, 45.75, 38.65, 34.66, 32.14, 28.91. LC-MS, Rt=2.847 min, [M-Cl]*=463.0. HRMS (ESI) m/z calcd. for C₂₁H₂₁C₃N₄S₂ [M−Cl]+=463.0580, found 463.0538.

In another particular embodiment, the invention may include the compound: (6-Chloro-2-(6-chlorobenzo[d]thiazol-2-yl)-2,3,4,9-tetrahydro-1H-pyrido[3,4-b]indol-4-yl)methanamine (12b) The mixture of iodinated intermediate (30 mg, 0.058 mmol, prepared in the synthesis of 12a) and aqueous ammonium hydroxyide (0.5 mL) in tert-Butyl alcohol (0.5 mL) in a sealed vial was heated at 120° C. for 2 h. The reaction mixture was concentrated and purified by silica gel flash chromatography to afford the title compound 12b as a white solid (21 mg, 89%). ¹H NMR (500 MHz, Methanol-d₄) δ 7.86 (d, J=3.3 Hz, 1H), 7.59 (dd, J=14.7, 2.2 Hz, 1H), 7.51 (dd, J=5.1, 2.1 Hz, 1H), 7.36 (t, J=8.8 Hz, 1H), 7.25 (dd, J=8.6, 5.1 Hz, 1H), 7.22-7.15 (m, 1H), 7.01 (ddd, J=8.6, 4.6, 2.1 Hz, 1H), 4.90 (d, J=11.8 Hz, 1H), 4.58 (ddd, J=15.9, 13.5, 1.4 Hz, 1H), 4.28 (td, J=13.2, 2.7 Hz, 1H), 3.61 (td, J=13.4, 3.7 Hz, 1H), 3.11 (td, J=8.0, 3.9 Hz, 1H), 2.95 (ddd, J=13.1, 6.8, 4.8 Hz, 1H), 2.71 (ddd, J=13.2, 8.8, 6.5 Hz, 1H).¹³C NMR (101 MHz, Methanol-d₄) δ 171.06, 152.26, 136.50, 132.80, 128.77, 127.69, 127.54, 125.99, 122.62, 121.60, 120.39, 118.38, 113.41, 110.11, 79.45, 50.19, 47.01, 45.17, 37.04. LC-MS, Rt=7.310 min, [M+H]⁺=403.0. HRMS (ESI) m/z calcd. for C₁₉H₁₇Cl₂N₄S [M+H]⁺=403.0540, found 403.0558.

In another particular embodiment, the invention may include the compound: 1-(6-Chloro-2-(6-chlorobenzo[d]thiazol-2-yl)-2,3,4,9-tetrahydro-1H-pyrido[3,4-b]indol-4-yl)-N,N-Dimethylmethanamine (12c) Prepared by the same method as 12a with a yield of 30%. ¹H NMR (500 MHz, Chloroform-d) δ 8.24 (s, 1H), 7.61 (d, J=2.1 Hz, 1H), 7.49 (d, J=2.0 Hz, 1H), 7.45 (d, J=8.6 Hz, 1H), 7.27 (d, J=3.2 Hz, 1H), 7.26-7.22 (m, 2H), 7.14 (dd, J=8.6, 2.0 Hz, 1H), 5.33 (d, J=16.3 Hz, 1H), 4.64 (dd, J=16.3, 1.5 Hz, 1H), 4.26 (dd, J=13.1, 2.3 Hz, 1H), 3.83-3.62 (m, 1H), 3.24 (dt, J=11.5, 2.7 Hz, 1H), 2.57-2.43 (m, 2H), 2.40 (s, 6H). ¹³C NMR (101 MHz, Chloroform-d) δ 169.69, 151.25, 134.76, 132.08, 131.42, 127.57, 126.71, 126.62, 125.68, 122.43, 120.61, 119.67, 117.88, 112.23, 110.68, 61.86, 50.69, 46.23, 45.09, 31.87. LC-MS, Rt=7.395 min, [M+H]⁺=431.0. HRMS (ESI) m/z calcd. for C₂₁H₂₁Cl₂N₄S [M+H]⁺=431.0853, found 431.0835.

In another particular embodiment, the invention may include the compound: 1-(6-Chloro-2-(6-chlorobenzo[d]thiazol-2-yl)-2,3,4,9-tetrahydro-1H-pyrido[3,4-b]indol-4-yl)-N,N,N-trimethylmethanaminium iodide (12d) The mixture of 1-(6-chloro-2-(6-chlorobenzo[d]thiazol-2-yl)-2,3,4,9-tetrahydro-1H-pyrido[3,4-b]indol-4-yl)-N,N-Dimethylmethanamine 12c (5.0 mg, 0.012 mmol) in CH₃I (0.2 mL) and MeOH (0.2 mL) was stirred at room temperature overnight. The reaction mixture was concentrated and dried under high vacuum to afford the title compound 12d (6.6 mg, 99%). ¹H NMR (500 MHz, Methanol-d₄) δ 7.74 (d, J=2.2 Hz, 1H), 7.52 (d, J=2.0 Hz, 1H), 7.49 (d, J=8.6 Hz, 1H), 7.35 (d, J=8.7 Hz, 1H), 7.30 (dd, J=8.6, 2.2 Hz, 1H), 7.12 (dd, J=8.6, 2.0 Hz, 1H), 4.93-4.89 (m, 1H), 4.69-4.64 (m, 1H), 3.91 (d, J=8.7 Hz, 1H), 3.74 (ddd, J=14.0, 3.1, 1.3 Hz, 1H), 3.68-3.61 (m, 1H), 3.53 (dd, J=5.5, 4.1 Hz, 1H), 3.43 (dt, J=14.0, 1.6 Hz, 1H), 3.39 (s, 9H). ¹³C NMR (101 MHz, Methanol-d₄) δ 178.58, 166.61, 159.86, 144.10, 142.16, 140.90, 136.64, 135.94, 134.82, 133.17, 130.63, 128.88, 127.03, 122.32, 117.00, 81.68, 80.04, 75.88, 69.69, 41.62. LC-MS, Rt=6.434 min, [M−I]⁺=445.2. HRMS (ESI) m/z calcd. for C₂₂H₂₃C₁₂N₄S [M+H]⁺=446.1094, found 446.1095.

In another particular embodiment, the invention may include the compound: N₁-((6-chloro-2-(6-chlorobenzo[d]thiazol-2-yl)-2,3,4,9-tetrahydro-1H-pyrido[3,4-b]indol-4-yl)methyl)ethane-1,2-diamine (12e) The iodinated intermediate (15 mg, 0.029 mmol prepared in the synthesis of 12a) and ethylenediamine (0.1 mL, 50.0 equiv) in tert-Butyl alcohol (0.5 mL) in a sealed tube were heated to 120° C. for 1 h. The mixture was then concentrated and purified via silica gel flash chromatography to afford the title compound 12e (10.0 mg, 77%). ¹H NMR (500 MHz, Methanol-d₄) δ 7.68 (t, J=2.4 Hz, 1H), 7.54 (t, J=2.4 Hz, 1H), 7.43 (dd, J=9.4, 3.3 Hz, 1H), 7.34-7.24 (m, 2H), 7.06 (dt, J=8.5, 2.5 Hz, 1H), 5.03 (d, J=15.7 Hz, 1H), 4.63 (d, J=16.2 Hz, 1H), 4.41 (d, J=13.4 Hz, 1H), 3.72-3.63 (m, 1H), 3.27-3.19 (m, 1H), 2.92 (t, J=10.9 Hz, 1H), 2.80 (d, J=13.0 Hz, 3H), 2.73-2.60 (m, 2H). LC-MS, Rt=5.676 min, [M+H]⁺=446.1. HRMS (ESI) m/z calcd. for C₂₁H₂₂Cl₂N₅S [M+H]⁺=446.0962, found 446.1003.

In another particular embodiment, the invention may include the compound: N-((6-chloro-2-(6-chlorobenzo[d]thiazol-2-yl)-2,3,4,9-tetrahydro-1H-pyrido[3,4-b]indol-4-yl)methyl)formimidamide (12f) To the mixture of (6-chloro-2-(6-chlorobenzo[d]thiazol-2-yl)-2,3,4,9-tetrahydro-1H-pyrido[3,4-b]indol-4-yl)methanamine 12b (7.6 mg, 0.019 mmol) and DIEA (16.5 μL, 0.095 mmol) at −55° C. was added ethyl formimidate hydrochloride (3.2 mg, 0.0285 mmol) in one portion. The reaction mixture was stirred at −55° C. for 1 h. After warming up to room temperature, the mixture was concentrated and purified by silica gel flash chromatography (DCM/MeOH=50:1 to 10:1) to afford title compound 12f (4.0 mg, 49%). ¹H NMR (500 MHz, Methanol-d₄) δ 7.94 (s, 1H), 7.84 (s, 1H), 7.70 (t, J=1.8 Hz, 1H), 7.52 (dd, J=11.5, 2.0 Hz, 1H), 7.43 (d, J=8.7 Hz, 1H), 7.31 (d, J=8.6 Hz, 1H), 7.27 (dd, J=8.7, 2.2 Hz, 1H), 7.06 (dd, J=8.6, 2.0 Hz, 1H), 5.04 (d, J=16.1 Hz, 1H), 4.67 (d, J=16.0 Hz, 1H), 3.75 (dd, J=13.7, 3.1 Hz, 1H), 3.60-3.46 (m, 3H), 3.03 (d, J=7.4 Hz, 1H). LC-MS, Rt=6.737 min, [M+H]⁺=430.1. HRMS (ESI) m/z calcd. for C₂₀H₁₈Cl₂N₅S [M+H]⁺=430.0649, found 430.0692.

In another particular embodiment, the invention may include the compound: 1-((6-chloro-2-(6-chlorobenzo[d]thiazol-2-yl)-2,3,4,9-tetrahydro-1H-pyrido[3,4-b]indol-4-yl)methyl)guanidine hydrochloride (12g) The mixture of (6-chloro-2-(6-chlorobenzo[d]thiazol-2-yl)-2,3,4,9-tetrahydro-1H-pyrido[3,4-b]indol-4-yl)methanamine 12b (42 mg, 0.104 mmol) and N,N′-Di-Boc-1H-pyrazole-1-carboxamidine (35 mg, 0.115 mmol) in anhydrous DCM (3 mL) was stirred at room temperature for 2 h. The mixture was concentrated and purified via silica gel flash chromatography to afford the guanidinylated intermediate (42 mg, 62%). ¹H NMR (500 MHz, Chloroform-d) δ 11.45 (s, 1H), 8.74 (t, J=5.6 Hz, 1H), 8.22 (s, 1H), 7.70 (d, J=1.9 Hz, 1H), 7.59 (d, J=2.1 Hz, 1H), 7.51 (d, J=8.6 Hz, 1H), 7.26 (d, J=2.2 Hz, 1H), 7.23 (d, J=8.6 Hz, 1H), 7.11 (dd, J=8.6, 2.0 Hz, 1H), 5.10 (d, J=16.1 Hz, 1H), 4.61 (dd, J=16.1, 1.4 Hz, 1H), 4.24 (d, J=13.4 Hz, 1H), 3.90 (dt, J=13.5, 5.4 Hz, 1H), 3.64 (dd, J=13.4, 3.8 Hz, 1H), 3.55 (d, J=4.3 Hz, 1H), 3.43 (ddd, J=13.8, 8.4, 5.6 Hz, 1H), 1.53 (d, J=1.4 Hz, 9H), 1.50 (s, 9H). HRMS (ESI) m/z calcd. for C₃₀H₃₅Cl₂N₆O₄S [M+H]⁺=645.1807, found 645.1794. Then the N-Boc intermediate (38 mg) was dissolved in HCl in 1,4-dioxane solution (1 mL, 4 M) in a sealed tube and heated to 80° C. overnight. The mixture was concentrated and dried over high vacuum to afford the title compound 12g as a white solid (28 mg, 99%). ¹H NMR (500 MHz, DMSO-d₆) δ 11.29 (s, 1H), 7.74 (d, J=2.2 Hz, 1H), 7.72-7.65 (m, 1H), 7.41 (d, J=2.0 Hz, 1H), 7.28 (d, J=8.6 Hz, 1H), 7.16 (d, J=8.6 Hz, 1H), 7.11 (dd, J=8.6, 2.2 Hz, 1H), 6.85 (dd, J=8.6, 2.1 Hz, 1H), 4.85 (d, J=16.4 Hz, 1H), 4.47 (d, J=16.3 Hz, 1H), 3.57 (dd, J=13.5, 3.6 Hz, 1H), 3.50-3.40 (m, 1H), 3.28-3.15 (m, 2H), 3.10 (dt, J=12.2, 6.8 Hz, 1H). ¹³C NMR (101 MHz, DMSO-d₆) δ 169.09, 157.13, 150.37, 134.62, 132.67, 131.42, 127.15, 126.46, 125.34, 123.69, 121.14, 121.08, 119.40, 117.54, 112.83, 107.51, 49.41, 45.66, 43.91, 43.69. LC-MS, Rₜ=4.888 min, [M−Cl]⁺=445.0. HRMS (ESI) m/z calcd. for C₂₀H₁₉Cl₂N₆S [M−Cl]⁺=445.0758, found 445.0777.

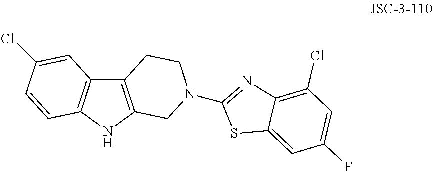 [SMILES: Fc1cc(Cl)c2nc(N3CCc4c([nH]c5ccc(Cl)cc45)C3)sc2c1]

In another particular embodiment, the invention may include the compound: 6-chloro-2-(4-chloro-6-fluoro-1,3-benzothiazol-2-yl)-2,3,4,9-tetrahydro-1H-pyrido[3,4-b]indole Prepared by method B using 2-chloro-4-fluoro-1-isothiocyanato-benzene as reactant. Pale yellow solid was obtained with a yield of 90%. ¹H NMR (400 MHz, Chloroform-d) δ 7.94 (s, 1H), 7.43 (d, J=2.0 Hz, 1H), 7.27-7.21 (m, 2H), 7.11 (ddd, J=8.9, 6.4, 2.3 Hz, 2H), 4.95 (s, 2H), 3.91 (t, J=5.7 Hz, 2H), 2.93 (t, J=5.8 Hz, 2H). LC-MS, Rt=8.948 min, [M+H]⁺=392.0.

### Example 10. General Procedure of the Synthesis Tryptoline-Based Benzothiazole Derivatives (Method A2)

The mixture of methyl 6-chloro-2,3,4,9-tetrahydro-1H-pyrido[3,4-b]indole-4-carboxylate (1.0 equiv), K₂CO₃ (5.0 equiv) and benzo[d]thiazole (1.5 equiv) in DMF (0.2 mmol/ml) under Argon were heated to 80° C. overnight. Then diluted with EA and washed with water three times. Dried over Na₂SO₄ and concentrated and purified with silica gel (hex/EA 10:1 to 1:1) to provide the desired white solid 10 (yield 60%-85%).

### Example 11. General Procedure of the Synthesis Amide of Tryptoline-Based Benzothiazole Derivitives (Method B2)

The ester (20 mg, 0.046 mmol) and lithium hydroxide (20 mg, 0.46 mmol) were dissolved in H₂O (1 ml, v:v=1:1), and stirred at room temperature overnight. TLC checked the starting materials almost gone. Quenched with 1 N HCl to adjust the solution to pH around 5, extracted with ethyl estate, the combined organic phase dried over Na₂SO₄, concentrated under reduced pressure to afford the desired acid product (20 mg, 99%). The acid (21 mg, 0.05 mmol) was dissolved in anhydrous DCM (1 ml), thionyl chloride (8.6 ul, 0.1 mmol) was added into the reaction mixture and 3 drops DMF was then added, the mixture was stirred at room temperature for 2h, then the free amine (26 ul, 0.25 mmol) was added, stirred at room temperature for another 2h. Diluted with ethyl acetate, washed with water and brine, dried over Na₂SO₄, concentrated and purified by flash chromatography (hex/EA=1:1 to 1:2) to afford the desired product (yield 50% to 90%).

### Example 12. General Procedure of the Synthesis Carbamate of Tryptoline-Based Benzothiazoles (Method C)

The mixture of alcohol (40 mg, 0.1 mmol) and CDI (20 mg, 0.12 mmol) in anhydrous DCM (1 ml) was stirred at room temperature for 4 h. After removed all the solvent, purified by flash chromatography to afford the intermediate (30 mg, 60%). The intermediate (1.0 equiv) reacted with amine (10 equiv) in anhydrous THF at room temperature for 2h. After removed all the solvent, purified by flash chromatography to afford the carbamate (yield 75-90%).

### Example 13: Exemplary Tryptoline-Based Benzothiazole Derivatives and their Synthesis

Exemplary Compounds Include:

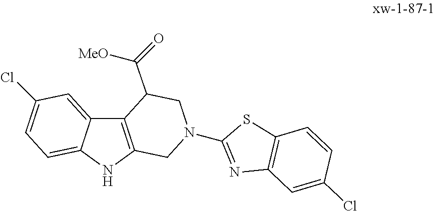 [SMILES: COC(=O)C1CN(c2nc3cc(Cl)ccc3s2)Cc2[nH]c3ccc(Cl)cc3c21]

In another particular embodiment, the invention may include the compound: Methyl 6-chloro-2-(5-chlorobenzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (45 mg, 0.15 mmol) and 2,5-dichlorobenzo[d]thiazole (46 mg, 0.225 mmol), K₂CO₃ (103 mg, 0.7475 mmol) in 1 ml DMF to afford desired product (47.8 mg, yield 74%). ¹H NMR (500 MHz, Chloroform-d) δ 8.18 (s, 1H), 7.55 (dt, J=10.1, 1.4 Hz, 2H), 7.51 (dd, J=8.4, 1.0 Hz, 1H), 7.24 (d, J=8.7 Hz, 1H), 7.20-7.11 (m, 1H), 7.08 (dd, J=8.4, 1.1 Hz, 1H), 5.18 (d, J=16.3 Hz, 1H), 4.73-4.59 (m, 1H), 4.43 (d, J=3.3 Hz, 1H), 4.04 (s, 1H), 3.95-3.81 (m, 1H), 3.74 (d, J=1.0 Hz, 3H). LC-MS, Rt=5.719 min, [M+H]⁺=432.0.

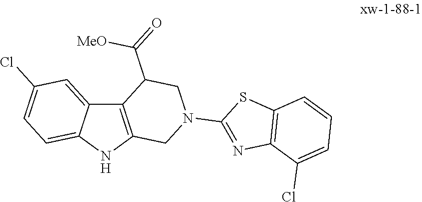 [SMILES: COC(=O)C1CN(c2nc3c(Cl)cccc3s2)Cc2[nH]c3ccc(Cl)cc3c21]

In another particular embodiment, the invention may include the compound: Methyl 6-chloro-2-(4-chlorobenzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (45 mg, 0.15 mmol) and 2,4-dichlorobenzo[d]thiazole (46 mg, 0.225 mmol), K₂CO₃ (103 mg, 0.7475 mmol) in 1 ml DMF to afford desired product (38.1 mg, yield 60%). ¹H NMR (500 MHz, Chloroform-d) δ 8.52 (s, 1H), 7.49 (d, J=1.9 Hz, 1H), 7.48-7.39 (m, 1H), 7.35-7.28 (m, 1H), 7.12 (d, J=8.6 Hz, 1H), 7.06 (dd, J=8.6, 2.0 Hz, 1H), 6.99 (t, J=7.9 Hz, 1H), 5.15 (d, J=16.6 Hz, 1H), 4.57 (dd, J=16.6, 1.7 Hz, 1H), 4.42 (dd, J=13.5, 3.1 Hz, 1H), 3.99 (d, J=3.8 Hz, 1H), 3.83 (dd, J=13.5, 4.5 Hz, 1H), 3.74 (s, 3H). LC-MS, T_{R}=5.412 min, [M+H]⁺=432.0.

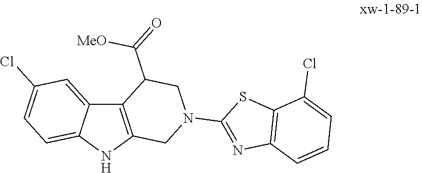 [SMILES: COC(=O)C1CN(c2nc3cccc(Cl)c3s2)Cc2[nH]c3ccc(Cl)cc3c21]

In another particular embodiment, the invention may include the compound: Methyl 6-chloro-2-(7-chlorobenzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 2,7-dichlorobenzo[d]thiazole (20 mg, 0.09975 mmol), K₂CO₃ (46 mg, 0.332 mmol) in 0.5 ml DMF to afford desired product (20 mg, yield 69%). ¹H NMR (500 MHz, Chloroform-d) δ 8.87 (s, 1H), 7.53 (d, J=2.7 Hz, 1H), 7.45-7.32 (m, 1H), 7.21 (td, J=8.1, 2.2 Hz, 1H), 7.17-7.11 (m, 1H), 7.07 (d, J=9.0 Hz, 2H), 5.10 (d, J=16.2 Hz, 1H), 4.63 (d, J=16.2 Hz, 1H), 4.44 (dd, J=13.1, 3.0 Hz, 1H), 4.04 (s, 1H), 3.91-3.82 (m, 1H), 3.75 (d, J=2.3 Hz, 3H). LC-MS, Rt=5.714 min, [M+H]⁺=432.0.

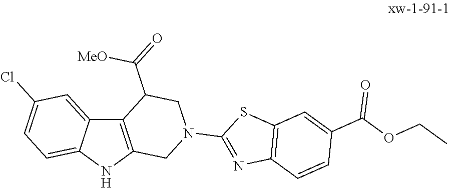 [SMILES: CCOC(=O)c1ccc2nc(N3Cc4[nH]c5ccc(Cl)cc5c4C(C(=O)OC)C3)sc2c1]

In another particular embodiment, the invention may include the compound: Ethyl 2-(6-chloro-4-(methoxycarbonyl)-1,3,4,9-tetrahydro-2H-pyrido[3,4-b]indol-2-yl)benzo[d]thiazole-6-carboxylate. Prepared by method A2 using methyl 6-chloro-2,3,4,9-tetrahydro-1H-pyrido[3,4-b]indole-4-carboxylate (45 mg, 0.15 mmol) and ethyl 2-chlorobenzo[d]thiazole-6-carboxylate (54 mg, 0.225 mmol), K₂CO₃ (103 mg, 0.7475 mmol) in 1 ml DMF to afford desired product (35.1 mg, yield 50%). ¹H NMR (500 MHz, Chloroform-d) δ 8.36 (t, J=1.4 Hz, 1H), 8.20 (s, 1H), 8.03 (dd, J=8.5, 1.5 Hz, 1H), 7.64-7.57 (m, 2H), 7.29-7.26 (m, 1H), 7.17 (dt, J=8.6, 1.5 Hz, 1H), 5.26 (d, J=16.3 Hz, 1H), 4.75 (dd, J=16.4, 1.5 Hz, 1H), 4.51 (dd, J=13.4, 3.3 Hz, 1H), 4.40 (dt, J=7.9, 6.6 Hz, 2H), 4.08 (d, J=3.9 Hz, 1H), 3.93 (dd, J=13.4, 4.4 Hz, 1H), 3.75 (d, J=1.1 Hz, 3H), 1.43 (td, J=7.1, 1.0 Hz, 3H). LC-MS, Rt=5.845 min, [M+H]⁺=470.1.

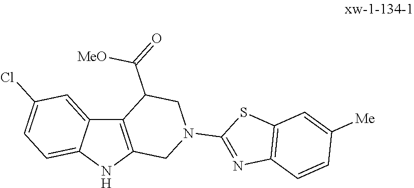 [SMILES: COC(=O)C1CN(c2nc3ccc(C)cc3s2)Cc2[nH]c3ccc(Cl)cc3c21]

In another particular embodiment, the invention may include the compound: Methyl 6-chloro-2-(6-methylbenzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 2-chloro-6-methylbenzo[d]thiazole (18 mg, 0.0996 mmol), K₂CO₃ (46 mg, 0.332 mmol) in 0.5 ml DMF to afford desired product (5.3 mg, yield 19%). ¹H NMR (500 MHz, Chloroform-d) δ 8.45 (s, 1H), 7.55 (s, 1H), 7.54-7.49 (m, 1H), 7.43 (s, 1H), 7.13 (d, J=7.1 Hz, 2H), 5.24 (d, J=15.1 Hz, 1H), 4.76 (d, J=15.1 Hz, 1H), 4.40 (d, J=12.5 Hz, 1H), 4.05 (s, 1H), 3.88 (d, J=12.2 Hz, 1H), 3.73 (s, 3H), 2.41 (s, 3H). LC-MS, Rt=5.516 min, [M+H]⁺=412.1.

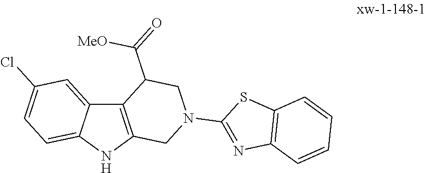 [SMILES: COC(=O)C1CN(c2nc3ccccc3s2)Cc2[nH]c3ccc(Cl)cc3c21]

In another particular embodiment, the invention may include the compound: Methyl 2-(benzo[d]thiazol-2-yl)-6-chloro-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 2-chlorobenzo[d]thiazole (18 mg, 0.0996 mmol), K₂CO₃ (46 mg, 0.332 mmol) in 0.5 ml DMF to afford desired product (5.1 mg, yield 19%). ¹H NMR (500 MHz, Chloroform-d) δ 7.77 (d, J=8.1 Hz, 1H), 7.60 (d, J=7.9 Hz, 1H), 7.52 (s, 1H), 7.40-7.32 (m, 1H), 7.26-7.23 (m, 3H), 7.23-7.17 (m, 1H), 7.10 (dd, J=8.6, 2.0 Hz, 1H), 5.50 (d, J=16.4 Hz, 1H), 4.90 (d, J=16.4 Hz, 1H), 4.40 (dd, J=12.8, 2.6 Hz, 1H), 4.02 (s, 1H), 3.93 (d, J=13.0 Hz, 1H). LC-MS, Rt=5.326 min, [M+H]⁺=398.0.

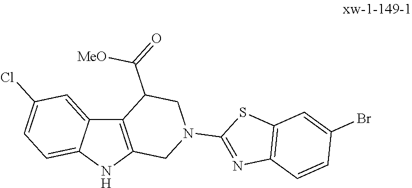 [SMILES: COC(=O)C1CN(c2nc3ccc(Br)cc3s2)Cc2[nH]c3ccc(Cl)cc3c21]

In another particular embodiment, the invention may include the compound: Methyl 2-(6-bromobenzo[d]thiazol-2-yl)-6-chloro-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 6-bromo-2-chlorobenzo[d]thiazole (25 mg, 0.0996 mmol), K₂CO₃ (46 mg, 0.332 mmol) in 0.5 ml DMF to afford desired product (20.9 mg, yield 63%). ¹H NMR (500 MHz, Chloroform-d) δ 8.51 (s, 1H), 7.73 (dd, J=1.8, 0.7 Hz, 1H), 7.57 (d, J=2.0 Hz, 1H), 7.44-7.35 (m, 2H), 7.20 (d, J=8.6 Hz, 1H), 7.13 (dd, J=8.6, 2.0 Hz, 1H), 5.15 (d, J=16.3 Hz, 1H), 4.67 (dd, J=16.3, 1.6 Hz, 1H), 4.44 (dd, J=13.4, 3.3 Hz, 1H), 4.11-3.99 (m, 1H), 3.87 (dd, J=13.3, 4.4 Hz, 1H), 3.76 (s, 3H). LC-MS, Rt=5.788 min, [M+H]⁺=478.0.

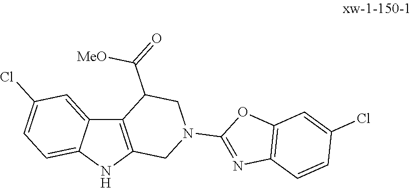 [SMILES: COC(=O)C1CN(c2nc3ccc(Cl)cc3o2)Cc2[nH]c3ccc(Cl)cc3c21]

In another particular embodiment, the invention may include the compound: Methyl 6-chloro-2-(6-chlorobenzo[d]oxazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 2,6-dichlorobenzo[d]oxazole (19 mg, 0.0996 mmol), K₂CO₃ (46 mg, 0.332 mmol) in 0.5 ml DMF to afford desired product (6.7 mg, yield 24%). ¹H NMR (500 MHz, Chloroform-d) δ 9.26 (s, 1H), 7.50 (s, 1H), 7.40-7.33 (m, 2H), 7.30 (d, J=8.7 Hz, 1H), 7.25 (d, J=1.9 Hz, 1H), 7.14 (dd, J=8.6, 2.0 Hz, 1H), 5.39 (d, J=15.9 Hz, 1H), 4.92 (d, J=15.8 Hz, 1H), 4.80 (dd, J=13.5, 2.7 Hz, 1H), 4.02 (s, 1H), 3.90 (d, J=13.4 Hz, 1H), 3.68 (s, 3H). LC-MS, Rt=5.330 min, [M+H]⁺=416.0.

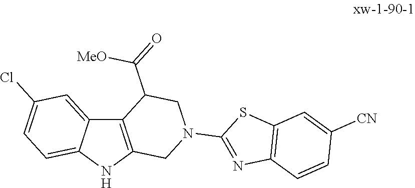 [SMILES: COC(=O)C1CN(c2nc3ccc(C#N)cc3s2)Cc2[nH]c3ccc(Cl)cc3c21]

In another particular embodiment, the invention may include the compound: Methyl 6-chloro-2-(6-cyanobenzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (45 mg, 0.15 mmol) and 2-chlorobenzo[d]thiazole-6-carbonitrile (48 mg, 0.225 mmol), K₂CO₃ (103 mg, 0.7475 mmol) in 1 ml DMF to afford desired product (33.0 mg, yield 52%). ¹H NMR (500 MHz, Chloroform-d) δ 8.07 (s, 1H), 7.93 (d, J=1.2 Hz, 1H), 7.64-7.54 (m, 3H), 7.31 (d, J=8.6 Hz, 1H), 7.20 (dd, J=8.6, 1.9 Hz, 1H), 5.34-5.26 (m, 1H), 4.75 (dd, J=16.5, 1.5 Hz, 1H), 4.52 (d, J=12.8 Hz, 1H), 4.08 (s, 1H), 3.92 (dd, J=13.4, 4.4 Hz, 1H), 3.75 (d, J=1.0 Hz, 3H). LC-MS, Rt=0.476 min, [M+H]⁺=423.1.

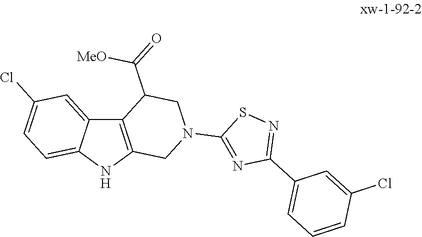 [SMILES: COC(=O)C1CN(c2nc(-c3cccc(Cl)c3)ns2)Cc2[nH]c3ccc(Cl)cc3c21]

In another particular embodiment, the invention may include the compound: Methyl 6-chloro-2-(3-(3-chlorophenyl)-1,2,4-thiadiazol-5-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (45 mg, 0.15 mmol) and 5-chloro-3-(3-chlorophenyl)-1,2,4-thiadiazole (52.0 mg, 0.225 mmol), K₂CO₃ (0.75 mmol, 103.5 mg) in 1 mL DMF to afford the title product (30 mg, 43%). ¹H NMR (500 MHz, Chloroform-d) δ 8.24 (d, J=1.9 Hz, 1H), 8.12 (dd, J=7.1, 1.9 Hz, 1H), 8.05 (s, 1H), 7.64 (d, J=7.8 Hz, 1H), 7.43-7.37 (m, 2H), 7.25 (t, J=7.5 Hz, 1H), 7.19 (t, J=7.4 Hz, 1H), 5.17 (d, J=18.7 Hz, 1H), 4.74 (d, J=16.0 Hz, 1H), 4.38 (d, J=12.9 Hz, 1H), 4.15 (d, J=4.0 Hz, 1H), 3.93 (dd, J=13.2, 4.5 Hz, 1H), 3.73 (s, 3H).

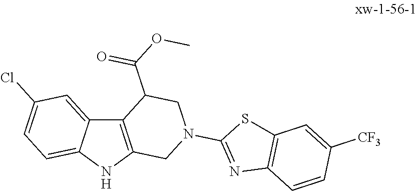 [SMILES: COC(=O)C1CN(c2nc3ccc(C(F)(F)F)cc3s2)Cc2[nH]c3ccc(Cl)cc3c21]

In another particular embodiment, the invention may include the compound: Methyl 6-chloro-2-(6-(trifluoromethyl)benzo[d]thiazol-2-yl)-2,3,4,9-tetrahydro-1H-pyrido[3,4-b]indole-4-carboxylate. Prepared by method A2 using methyl 6-chloro-2,3,4,9-tetrahydro-1H-pyrido[3,4-b]indole-4-carboxylate (20 mg, 0.0665 mmol) and 2-chloro-6-(trifluoromethyl)benzo[d]thiazole (24 mg, 0.099 mmol), K₂CO₃ (46 mg, 0.33 mmol) in 0.5 ml DMF to afford desired product (22.6 mg, yield 75%). ¹H NMR (500 MHz, Chloroform-d) δ 8.35 (s, 1H), 7.93-7.84 (m, 1H), 7.66-7.50 (m, 3H), 7.27-7.22 (m, 1H), 7.16 (dd, J=8.6, 2.0 Hz, 1H), 5.23 (d, J=16.3 Hz, 1H), 4.72 (dd, J=16.3, 1.6 Hz, 1H), 4.50 (dd, J=13.4, 3.2 Hz, 1H), 4.12-4.02 (m, 1H), 3.91 (dd, J=13.4, 4.4 Hz, 1H), 3.76 (s, 3H). LC-MS, Rt=9.164 min, [M+H]⁺=466.0.

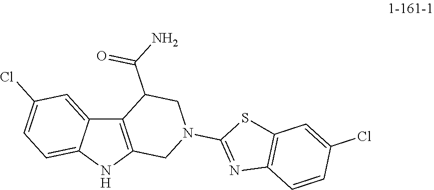 [SMILES: NC(=O)C1CN(c2nc3ccc(Cl)cc3s2)Cc2[nH]c3ccc(Cl)cc3c21]

In another particular embodiment, the invention may include the compound: 6-chloro-2-(6-chlorobenzo[d]thiazol-2-yl)-2,3,4,9-tetrahydro-1H-pyrido[3,4-b]indole-4-carboxamide. Prepared by the mixture of methyl 6-chloro-2-(6-(trifluoromethyl)benzo[d]thiazol-2-yl)-2,3,4,9-tetrahydro-1H-pyrido[3,4-b]indole-4-carboxylate and 2.0 M ammonium hydroxyl in MeOH reflux overnight. Purified by prepared TLC plate (DCM/MeOH=10:1) to afford the title compound (10.0 mg, 68%). ¹H NMR (500 MHz, DMSO-d₆) δ 11.32 (s, 1H), 7.95 (d, J=2.2 Hz, 1H), 7.58 (s, 1H), 7.47 (d, J=8.7 Hz, 1H), 7.40 (d, J=8.6 Hz, 1H), 7.31 (dd, J=8.6, 2.3 Hz, 1H), 7.23 (d, J=2.3 Hz, 1H), 7.08 (dd, J=8.6, 2.1 Hz, 1H), 5.03-4.74 (m, 2H), 4.05 (t, J=5.3 Hz, 2H), 3.92 (d, J=5.7 Hz, 1H). LC-MS, T_{R}=8.113 min, [M+H]⁺=417.0.

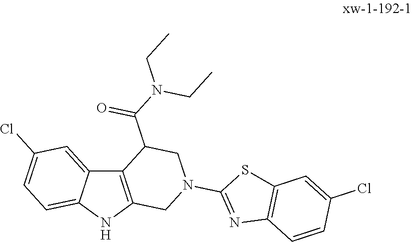 [SMILES: CCN(CC)C(=O)C1CN(c2nc3ccc(Cl)cc3s2)Cc2[nH]c3ccc(Cl)cc3c21]

In another particular embodiment, the invention may include the compound: 6-chloro-2-(6-chlorobenzo[d]thiazol-2-yl)-N,N-diethyl-2,3,4,9-tetrahydro-1H-pyrido[3,4-b]indole-4-carboxamide. Prepared by method B2 using the acid (21 mg, 0.05 mmol) and diethylamine (0.25 mmol, 0.026 ml) to afford the title compound (23 mg, 94%). ¹H NMR (500 MHz, Chloroform-d) δ 8.45 (s, 1H), 7.58 (d, J=2.1 Hz, 1H), 7.48 (d, J=8.6 Hz, 1H), 7.28 (s, 7H), 7.21 (d, J=2.0 Hz, 1H), 7.17 (d, J=8.6 Hz, 1H), 7.06 (dd, J=8.6, 2.0 Hz, 1H), 5.12 (d, J=16.0 Hz, 1H), 4.59 (d, J=16.2 Hz, 1H), 4.42 (dd, J=9.3, 4.8 Hz, 1H), 4.27 (dd, J=13.4, 4.8 Hz, 1H), 3.99 (s, 1H), 3.78 (dq, J=15.3, 7.3 Hz, 2H), 3.65 (dt, J=15.0, 7.3 Hz, 1H), 3.44 (dd, J=13.5, 6.9 Hz, 1H), 1.45 t, J=7.1 Hz, 3H), 1.33 t, J=7.1 Hz, 3H).

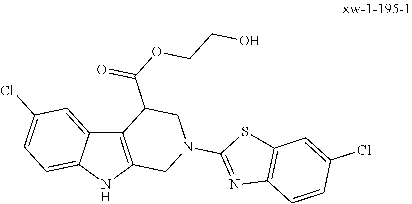 [SMILES: O=C(OCCO)C1CN(c2nc3ccc(Cl)cc3s2)Cc2[nH]c3ccc(Cl)cc3c21]

In another particular embodiment, the invention may include the compound: 2-hydroxyethyl 6-chloro-2-(6-chlorobenzo[d]thiazol-2-yl)-2,3,4,9-tetrahydro-1H-pyrido[3,4-b]indole-4-carboxylate Prepared by method B2 using the acid (21 mg, 0.05 mmol) and ethylene glycol (0.15 mmol, 0.0084 ml) to afford the title compound (16 mg, 67%). ¹H NMR (500 MHz, Chloroform-d) δ 8.72 (s, 1H), 7.56 (d, J=2.0 Hz, 1H), 7.49-7.38 (m, 2H), 7.25-7.20 (m, 1H), 7.05-6.87 (m, 2H), 4.80 (d, J=13.3 Hz, 1H), 4.64 (d, J=16.0 Hz, 1H), 4.47-4.33 (m, 2H), 4.22 (ddd, J=11.8, 5.9, 3.4 Hz, 1H), 4.01 (d, J=3.8 Hz, 1H), 3.83-3.77 (m, 2H), 3.74 (s, 1H), 3.66 (dd, J=13.3, 4.5 Hz, 1H). LC-MS, Rt=5.176 min, [M+H]⁺=462.0.

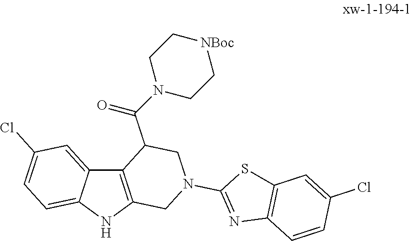 [SMILES: CC(C)(C)OC(=O)N1CCN(C(=O)C2CN(c3nc4ccc(Cl)cc4s3)Cc3[nH]c4ccc(Cl)cc4c32)CC1]

In another particular embodiment, the invention may include the compound: Tert-butyl 4-(6-chloro-2-(6-chlorobenzo[d]thiazol-2-yl)-2,3,4,9-tetrahydro-1H-pyrido[3,4-b]indole-4-carbonyl)piperazine-1-carboxylate. Prepared by method B2 using the acid (21 mg, 0.05 mmol) and tert-butyl piperazine-1-carboxylate (0.25 mmol, 47 mg) to afford the title compound (19.5 mg, 67%). ¹H NMR (500 MHz, DMSO-d₆) δ 11.27 (s, 1H), 7.95 (d, J=2.3 Hz, 1H), 7.42 (d, J=8.6 Hz, 1H), 7.38 (d, J=8.5 Hz, 1H), 7.30 (dd, J=8.6, 2.3 Hz, 1H), 7.25 (d, J=2.0 Hz, 1H), 7.06 (dd, J=8.6, 2.1 Hz, 1H), 4.95 (d, J=16.4 Hz, 1H), 4.86 (d, J=16.5 Hz, 1H), 4.54 (s, 1H), 4.13-3.95 (m, 2H), 3.83 (d, J=30.9 Hz, 1H), 3.60-3.50 (m, 1H), 3.39 (d, J=17.2 Hz, 2H), 3.30 (d, J=17.2 Hz, 2H), 1.45 (s, 9H). LC-MS, Rt=5.726 min, [M+H]⁺=586.1.

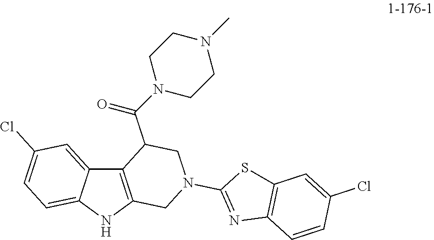 [SMILES: CN1CCN(C(=O)C2CN(c3nc4ccc(Cl)cc4s3)Cc3[nH]c4ccc(Cl)cc4c32)CC1]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)(4-methylpiperazin-1-yl)methanone. Prepared by method B2 using the acid (10 mg, 0.024 mmol) and tert-butyl piperazine-1-carboxylate (0.25 mmol, 47 mg) to afford the title compound (19.5 mg, 67%). ¹H NMR (500 MHz, DMSO-d₆) δ 11.27 (s, 1H), 7.95 (d, J=2.3 Hz, 1H), 7.45 (d, J=8.6 Hz, 1H), 7.39 (d, J=8.6 Hz, 1H), 7.31 (dd, J=8.6, 2.3 Hz, 1H), 7.25 (d, J=2.1 Hz, 1H), 7.06 (dd, J=8.6, 2.1 Hz, 1H), 4.96-4.83 (m, 2H), 4.56 (t, J=5.4 Hz, 1H), 4.08 (dd, J=13.4, 4.7 Hz, 1H), 3.98 (dd, J=13.7, 5.9 Hz, 1H), 3.82 (d, J=15.9 Hz, 2H), 3.50 (s, 2H), 3.37 (s, 4H), 2.28 (s, 3H). LC-MS, Rt=5.892 min, [M+H]⁺=500.1.

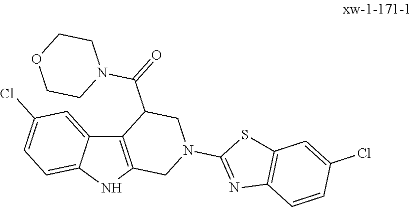 [SMILES: O=C(C1CN(c2nc3ccc(Cl)cc3s2)Cc2[nH]c3ccc(Cl)cc3c21)N1CCOCC1]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)(morpholino)methanone. Prepared by method B2 using the acid (15 mg, 0.036 mmol) and morpholine (0.108 mmol, 9.5 ul) to afford the title compound (10 mg, 57%). ¹H NMR (500 MHz, DMSO-d₆) δ 11.27 (s, 1H), 7.95 (d, J=2.2 Hz, 1H), 7.44 (d, J=8.6 Hz, 1H), 7.39 (d, J=8.6 Hz, 1H), 7.31 (dd, J=8.6, 2.3 Hz, 1H), 7.28 (d, J=2.1 Hz, 1H), 7.06 (dd, J=8.6, 2.1 Hz, 1H), 4.96 (d, J=16.4 Hz, 1H), 4.86 (d, J=16.4 Hz, 1H), 4.53 (t, J=5.1 Hz, 1H), 4.09 (d, J=4.4 Hz, 1H), 4.03 (d, J=5.5 Hz, 1H), 3.85 (s, 1H), 3.80 (s, 1H), 3.63 (s, 1H), 3.48 (s, 1H), 3.38 (s, 2H), 3.31 (s, 2H). LC-MS, Rt=7.044 min, [M+H]⁺=487.0.

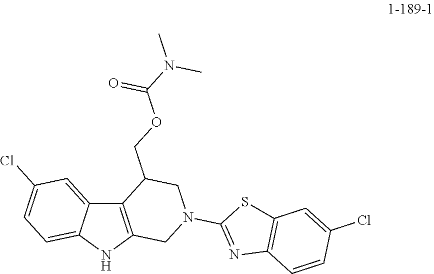 [SMILES: CN(C)C(=O)OCC1CN(c2nc3ccc(Cl)cc3s2)Cc2[nH]c3ccc(Cl)cc3c21]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)methyl dimethylcarbamate. Prepared by method C using the activated carbamate intermediate (10 mg, 0.02 mmol) and dimethylamine (0.2 mmol, 9 mg) to afford the title compound (7.8 mg, 82%). ¹H NMR (500 MHz, Chloroform-d) δ 8.42 (s, 1H), 7.58 (t, J=2.4 Hz, 2H), 7.46 (d, J=8.6 Hz, 1H), 7.30-7.24 (m, 2H), 7.21 (d, J=8.6 Hz, 1H), 7.10 (dd, J=8.6, 2.0 Hz, 1H), 5.24 (d, J=16.2 Hz, 1H), 4.60 (dd, J=16.3, 1.4 Hz, 1H), 4.51 (dd, J=11.1, 5.3 Hz, 1H), 4.16 (dd, J=13.3, 2.1 Hz, 1H), 4.00 (dd, J=11.1, 9.4 Hz, 1H), 3.70 (dd, J=13.3, 3.8 Hz, 1H), 3.51 (ddd, J=9.4, 4.7, 2.3 Hz, 1H), 3.01 (s, 3H), 2.97 (s, 3H). LC-MS, Rt=5.454 min, [M+H]+=475.1.

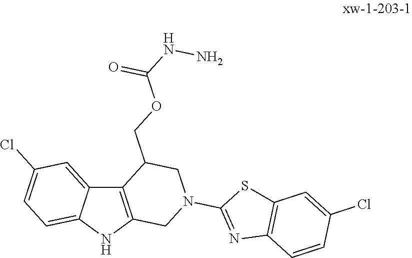 [SMILES: NNC(=O)OCC1CN(c2nc3ccc(Cl)cc3s2)Cc2[nH]c3ccc(Cl)cc3c21]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)methyl hydrazinecarboxylate. Prepared by method C using the activated carbamate intermediate (10 mg, 0.02 mmol) and hydrazine (0.2 mmol, 6.3 ul) to afford the title compound (6.2 mg, 67%). ¹H NMR (500 MHz, Chloroform-d) δ 8.23 (s, 1H), 7.58 (d, J=2.1 Hz, 2H), 7.45 (d, J=8.6 Hz, 1H), 7.29-7.21 (m, 2H), 7.13 (dd, J=8.6, 2.0 Hz, 1H), 6.14 (s, 1H), 5.22 (d, J=16.2 Hz, 1H), 4.59 (dd, J=16.1, 1.5 Hz, 1H), 4.51 (dd, J=11.0, 5.0 Hz, 1H), 4.14 (dd, J=15.8, 8.7 Hz, 1H), 4.09-4.00 (m, 1H), 3.81 (s, 1H), 3.70 (dd, J=13.4, 3.8 Hz, 1H), 3.47 (s, 1H). LC-MS, Rt=6.275 min, [M+H]⁺=404.0.

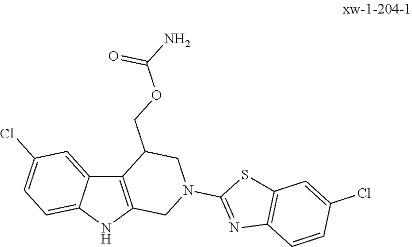 [SMILES: NC(=O)OCC1CN(c2nc3ccc(Cl)cc3s2)Cc2[nH]c3ccc(Cl)cc3c21]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)methyl carbamate. Prepared by method C using the activated carbamate intermediate (10 mg, 0.02 mmol) and ammonia solution (0.1 ml) in anhydrous THF (1 ml) to afford the title compound (8.0 mg, 88%). ¹H NMR (500 MHz, Methanol-d₄) δ 7.71 (d, J=2.0 Hz, 1H), 7.59 (d, J=2.0 Hz, 1H), 7.46 (d, J=8.6 Hz, 1H), 7.35-7.26 (m, 2H), 7.09 (d, J=2.1 Hz, 1H), 5.16 (d, J=16.3 Hz, 1H), 4.64 (dd, J=16.2, 1.5 Hz, 1H), 4.46 (dd, J=11.0, 4.3 Hz, 1H), 4.34-4.24 (m, 1H), 3.88 (dd, J=11.0, 10.1 Hz, 1H), 3.77 (dd, J=13.4, 3.8 Hz, 1H), 3.48 (d, J=10.0 Hz, 1H). LC-MS, Rt=7.220 min, [M+H]⁺=447.0.

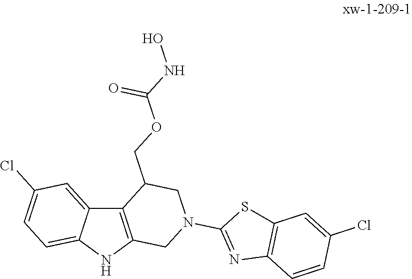 [SMILES: O=C(NO)OCC1CN(c2nc3ccc(Cl)cc3s2)Cc2[nH]c3ccc(Cl)cc3c21]

In another particular embodiment, the invention may include the compound: (6-chloro-2-(6-chlorobenzo[d]thiazol-2-yl)-2,3,4,9-tetrahydro-1H-pyrido[3,4-b]indol-4-yl)methyl hydroxycarbamate. Prepared by method C using the activated carbamate intermediate (10 mg, 0.02 mmol) and hydroxylamine hydrochloride (0.2 mmol, 13.9 mg) and triethylamine (28 ul, 0.2 mmol) to afford the title compound (5.0 mg, 54%). ¹H NMR (500 MHz, Methanol-d₄) δ 7.69 (d, J=2.1 Hz, 1H), 7.60 (d, J=2.0 Hz, 1H), 7.45 (d, J=8.5 Hz, 1H), 7.32-7.25 (m, 2H), 7.08 (dd, J=8.6, 2.0 Hz, 1H), 5.15 (d, J=16.2 Hz, 1H), 4.62 (dd, J=16.2, 1.5 Hz, 1H), 4.52 (ddd, J=11.0, 4.2, 1.1 Hz, 1H), 4.27 (dd, J=13.8, 2.2 Hz, 1H), 3.94 (t, J=10.6 Hz, 1H), 3.75 (dd, J=13.3, 3.7 Hz, 1H), 3.48 (d, J=10.0 Hz, 1H). LC-MS, Rt=4.935 min, [M+H]⁺=463.0.

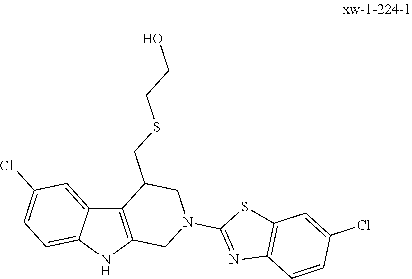 [SMILES: OCCSCC1CN(c2nc3ccc(Cl)cc3s2)Cc2[nH]c3ccc(Cl)cc3c21]

In another particular embodiment, the invention may include the compound: 2-(((6-chloro-2-(6-chlorobenzo[d]thiazol-2-yl)-2,3,4,9-tetrahydro-1H-pyrido[3,4-b]indol-4-yl)methyl)thio)ethan-1-ol. Prepared by method using the iodinated intermediate (9.6 mg, 0.018 mmol) and 2-Mercaptoethanol (0.1 ml, 80 equiv) and sodium hydroxide (2.2 mg, 0.054 mmol) in tert-Butyl alcohol to afford the title compound (5.0 mg, 60%). ¹H NMR (500 MHz, Chloroform-d) δ 8.16 (s, 1H), 7.61 (d, J=2.1 Hz, 1H), 7.51 (d, J=2.0 Hz, 1H), 7.49 (d, J=8.6 Hz, 1H), 7.31-7.25 (m, 2H), 7.16 (dd, J=8.6, 2.0 Hz, 1H), 5.22 (d, J=16.2 Hz, 1H), 4.63 (dd, J=16.2, 1.4 Hz, 1H), 4.46 (dd, J=13.3, 2.2 Hz, 1H), 3.80 (t, J=5.8 Hz, 2H), 3.73 (ddd, J=13.4, 3.8, 1.3 Hz, 1H), 3.28 (dt, J=11.0, 2.0 Hz, 1H), 3.07 (ddd, J=13.1, 3.6, 1.3 Hz, 1H), 2.86 (dd, J=6.1, 5.4 Hz, 2H), 2.67 (dd, J=13.2, 10.9 Hz, 1H), 2.46 (s, 1H). LC-MS, Rt=7.305 min, [M+H]⁺=464.0.

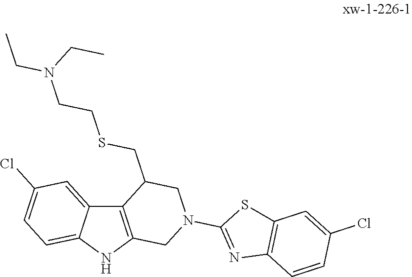 [SMILES: CCN(CC)CCSCC1CN(c2nc3ccc(Cl)cc3s2)Cc2[nH]c3ccc(Cl)cc3c21]

In another particular embodiment, the invention may include the compound: 2-(((6-chloro-2-(6-chlorobenzo[d]thiazol-2-yl)-2,3,4,9-tetrahydro-1H-pyrido[3,4-b]indol-4-yl)methyl)thio)-N,N-diethylethan-1-amine Prepared by method using the iodinated intermediate (10 mg, 0.019 mmol) and 2-(diethylamino)ethane-1-thiol (13 mg, 5.0 equiv) and sodium hydroxide (2.3 mg, 0.058 mmol) in tert-Butyl alcohol to afford the title compound (7.6 mg, 75%).¹H NMR (500 MHz, Chloroform-d) δ 8.30 (s, 1H), 7.60 (d, J=2.1 Hz, 1H), 7.52 (d, J=1.9 Hz, 1H), 7.46 (d, J=8.6 Hz, 1H), 7.28-7.25 (m, 2H), 7.15 (dd, J=8.6, 2.0 Hz, 1H), 5.30 (d, J=16.3 Hz, 1H), 4.63 (dd, J=16.3, 1.3 Hz, 1H), 4.34 (dd, J=13.3, 2.3 Hz, 1H), 3.74 (dd, J=13.3, 3.9 Hz, 1H), 3.32-3.23 (m, 1H), 3.06 (dd, J=13.3, 3.5 Hz, 1H), 2.76 (p, J=3.2, 2.6 Hz, 4H), 2.70-2.56 (m, 5H), 1.08 (t, J=7.2 Hz, 6H). LC-MS, Rt=7.506 min, [M+H]⁺=519.0.

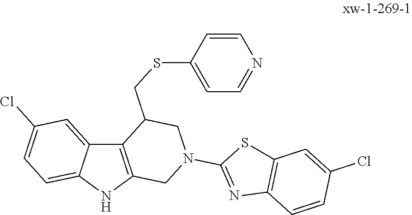 [SMILES: Clc1ccc2nc(N3Cc4[nH]c5ccc(Cl)cc5c4C(CSc4ccncc4)C3)sc2c1]

In another particular embodiment, the invention may include the compound: 6-chloro-2-(6-chloro-4-((pyridin-4-ylthio)methyl)-1,3506,4,9-tetrahydro-2H-pyrido[3,4-b]indol-2-yl)benzo[d]thiazole ¹H NMR (500 MHz, Chloroform-d) δ 8.86 (s, 1H), 8.44-8.39 (m, 2H), 7.59 (d, J=2.1 Hz, 1H), 7.53 (d, J=2.1 Hz, 1H), 7.46 (d, J=8.5 Hz, 1H), 7.28 (d, J=2.1 Hz, 1H), 7.26 (d, J=1.3 Hz, 1H), 7.24-7.21 (m, 2H), 7.17 (dd, J=8.6, 2.0 Hz, 1H), 5.29 (d, J=16.3 Hz, 1H), 4.63 (dd, J=16.2, 1.3 Hz, 1H), 4.34 (dd, J=13.4, 2.1 Hz, 1H), 3.77 (dd, J=13.4, 3.7 Hz, 1H), 3.50 (dd, J=13.3, 4.2 Hz, 1H), 3.41 (d, J=10.0 Hz, 1H), 3.14 (dd, J=13.3, 10.0 Hz, 1H). LC-MS, Rt=7.327 min, [M+H]+=497.0.

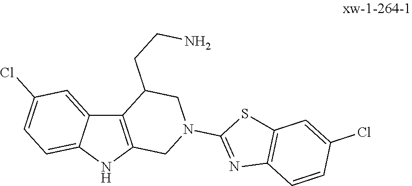 [SMILES: NCCC1CN(c2nc3ccc(Cl)cc3s2)Cc2[nH]c3ccc(Cl)cc3c21]

In another particular embodiment, the invention may include the compound: 2-(6-chloro-2-(6-chlorobenzo[d]thiazol-2-yl)-2,3,4,9-tetrahydro-1H-pyrido[3,4-b]indol-4-yl)ethan-1-amine Prepared by method using the mixture of iodinated intermediate (15 mg, 0.029 mmol) and Potassium cyanide (19 mg, 10.0 equiv) in DMSO at 120° C. for 2 h to afford the cyanide compound (8.8 mg, 73%), which was reduced by lithium Aluminum hydride (3.0 equiv) in anhydrous THF to afford the title compound(5.3 mg, 58%). ¹H NMR (500 MHz, Methanol-d₄) δ 7.56 (d, J=2.2 Hz, 1H), 7.43 (d, J=2.0 Hz, 1H), 7.33 (d, J=8.6 Hz, 1H), 7.21 (d, J=8.6 Hz, 1H), 7.16 (dd, J=8.6, 2.2 Hz, 1H), 6.96 (dd, J=8.6, 2.0 Hz, 1H), 4.91 (d, J=16.0 Hz, 1H), 4.51 (d, J=16.0 Hz, 1H), 4.12 (dd, J=13.4, 2.4 Hz, 1H), 3.61 (dd, J=13.4, 3.8 Hz, 1H), 3.21-3.14 (m, 1H), 3.07 (td, J=11.7, 5.3 Hz, 1H), 2.93 (td, J=12.0, 11.6, 5.8 Hz, 1H), 2.06-1.89 (m, 2H), 1.83 (s, 2H). ¹³C NMR (101 MHz, CD₃OD) S 169.53, 150.89, 135.10, 131.38, 131.19, 127.12, 126.38, 126.20, 124.64, 121.30, 120.24, 119.08, 116.89, 112.12, 109.65, 50.82, 45.46, 37.78, 31.83, 30.32. LC-MS Rt=7.387 min, [M+H]⁺=417.0.

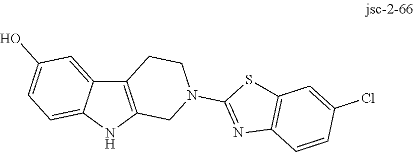 [SMILES: Oc1ccc2[nH]c3c(c2c1)CCN(c1nc2ccc(Cl)cc2s1)C3]

In another particular embodiment, the invention may include the compound: 2-(6-chloro-1,3-benzothiazol-2-yl)-2,3,4,9-tetrahydro-1H-pyrido[3,4-b]indol-6-ol Yellow solid was obtained with a yield of 40%. ¹H NMR (400 MHz, DMSO-d₆) δ 10.61 (s, 1H), 8.63 (d, J=0.9 Hz, 1H), 7.93 (d, J=2.3 Hz, 1H), 7.46 (d, J=8.6 Hz, 1H), 7.30 (dd, J=8.6, 2.2 Hz, 1H), 7.12 (d, J=8.6 Hz, 1H), 6.72 (d, J=2.3 Hz, 1H), 6.61-6.47 (m, 1H), 4.81 (s, 2H), 3.93 (t, J=5.7 Hz, 2H), 2.80 (t, J=5.3 Hz, 2H). LC-MS, Rt=6.646 min, [M+H]⁺=356.0.

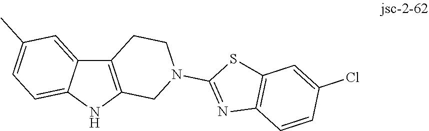 [SMILES: Cc1ccc2[nH]c3c(c2c1)CCN(c1nc2ccc(Cl)cc2s1)C3]

In another particular embodiment, the invention may include the compound: 2-(6-chloro-1,3-benzothiazol-2-yl)-6-methyl-2,3,4,9-tetrahydro-1H-pyrido[3,4-b]indole Yellow solid was obtained with a yield of 43%. ¹H NMR (400 MHz, Chloroform-d) δ 7.79 (s, 1H), 7.57 (d, J=2.1 Hz, 1H), 7.44 (d, J=8.6 Hz, 1H), 7.24 (s, 3H), 7.03-6.96 (m, 1H), 4.89 (s, 2H), 3.93 (t, J=5.7 Hz, 2H), 3.01-2.90 (m, 2H), 2.43 (s, 3H). LC-MS, Rt=8.041 min, [M+H]⁺=354.0.

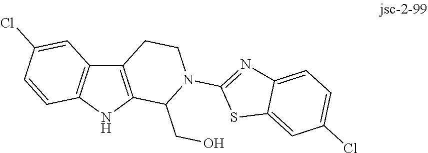 [SMILES: OCC1c2[nH]c3ccc(Cl)cc3c2CCN1c1nc2ccc(Cl)cc2s1]

In another particular embodiment, the invention may include the compound: [6-chloro-2-(6-chloro-1,3-benzothiazol-2-yl)-2,3,4,9-tetrahydro-1H-pyrido[3,4-b]indol-1-yl]methanol To a round bottom flask was added LiAlH₄ (12 mg, 0.300 mmol) and THF (2 mL), and the reaction mixture was cooled to 0° C. And the solution of starting marital (42 mg, 0.100 mmol) in THF (2 mL) was added into the reaction mixture dropwise, and was stirred for 4 h at rt. The reaction was quenched with 1 mL of H₂O, 1 mL of NaOH solution (10%), and then 3 mL of H₂O. Then ethyl acetate (10 mL) and H₂O (10 mL) were added into the mixture, and the aqueous layer was extract with ethyl acetate after separation two times. The organic layer then was combined and washed with brine. The solvents were removed under vacuum after dried with Na₂SO₄, and the residue was purified by flash chromatography on silica gel using ethyl acetate/hexane as eluent. Pale yellow solid was obtained with a yield of 50%. ¹H NMR (400 MHz, Chloroform-d) δ 8.59 (d, J=12.1 Hz, 1H), 7.63-7.53 (m, 1H), 7.44 (d, J=2.0 Hz, 1H), 7.31-7.28 (m, 1H), 7.22-7.28 (m, 1H), 7.12-7.08 (m, 2H), 5.60 (dt, J=14.4, 6.6 Hz, 1H), 4.18 (ddd, J=10.5, 5.9, 3.1 Hz, 1H), 3.94 (dd, J=10.5, 7.3 Hz, 1H), 3.83 (s, 1H), 3.74-3.66 (m, 1H), 3.67-3.60 (m, 1H), 3.03 (t, J=6.0 Hz, 1H), 2.82-2.76 (m, 2H). LC-MS, Rt=5.718 min, [M+H]⁺=404.0.

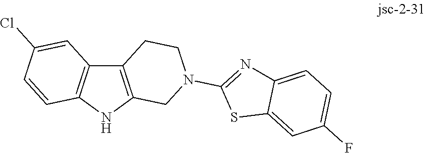 [SMILES: Fc1ccc2nc(N3CCc4c([nH]c5ccc(Cl)cc45)C3)sc2c1]

In another particular embodiment, the invention may include the compound: 6-chloro-2-(6-fluoro-1,3-benzothiazol-2-yl)-2,3,4,9-tetrahydro-1H-pyrido[3,4-b]indole (4t) Yellow solid was obtained with a yield of 45%. ¹H NMR (400 MHz, Chloroform-d) δ 8.02 (s, 1H), 7.52-7.44 (m, 2H), 7.34 (dd, J=1.1, 2.6 Hz, 1H), 7.25 (d, J=9.1 Hz, 1H), 7.13 (dd, J=8.6, 2.0 Hz, 1H), 7.08-6.95 (m, 1H), 4.92 (d, J=1.6 Hz, 2H), 3.93 (t, J=5.7 Hz, 2H), 3.06-2.84 (m, 2H). LC-MS, Rt=6.553 min, [M+H]⁺=358.0.

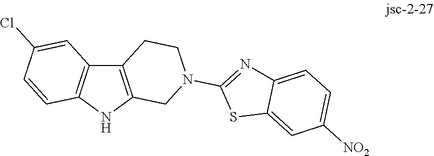 [SMILES: O=[N+]([O-])c1ccc2nc(N3CCc4c([nH]c5ccc(Cl)cc45)C3)sc2c1]

In another particular embodiment, the invention may include the compound: 6-chloro-2-(6-nitro-1,3-benzothiazol-2-yl)-2,3,4,9-tetrahydro-1H-pyrido[3,4-b]indole Yellow solid was obtained with a yield of 42%. ¹H NMR (400 MHz, DMSO-d₆) δ 11.19 (s, 1H), 8.86 (d, J=2.5 Hz, 1H), 8.63 (d, J=2.4 Hz, 1H), 7.57 (d, J=8.8 Hz, 1H), 7.49 (d, J=2.1 Hz, 1H), 7.38 (d, J=8.6 Hz, 1H), 7.07 (dd, J=8.6, 2.1 Hz, 1H), 4.97 (s, 2H), 4.04 (m, 2H), 2.97-2.86 (m, 2H).LC-MS, Rt=7.592 min, [M+H]⁺=385.0.

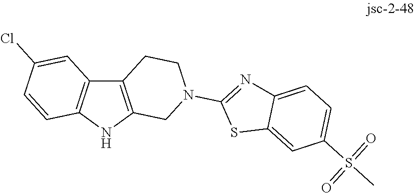 [SMILES: CS(=O)(=O)c1ccc2nc(N3CCc4c([nH]c5ccc(Cl)cc45)C3)sc2c1]

In another particular embodiment, the invention may include the compound: 6-chloro-2-[6-(methanesulfonyl)-1,3-benzothiazol-2-yl]-2,3,4,9-tetrahydro-1H-pyrido[3,4-b]indole Pale yellow solid was obtained with a yield of 44%. ¹H NMR (400 MHz, DMSO-d₆) δ 11.31 (s, 1H), 7.79 (dd, J=8.5, 2.0 Hz, 1H), 7.63 (d, J=8.5 Hz, 1H), 7.49 (d, J=2.1 Hz, 1H), 7.38 (d, J=8.6 Hz, 1H), 7.06 (dd, J=8.6, 2.1 Hz, 1H), 4.05-3.90 (m, 2H), 3.20 (s, 3H), 2.98-2.86 (m, 2H). LC-MS, Rt=5.732 min, [M+H]=418.0.

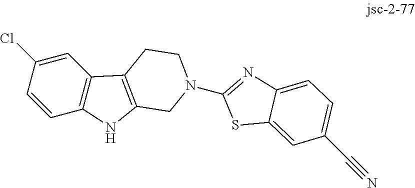 [SMILES: N#Cc1ccc2nc(N3CCc4c([nH]c5ccc(Cl)cc45)C3)sc2c1]

In another particular embodiment, the invention may include the compound: 2-(6-chloro-1,3,4,9-tetrahydro-2H-pyrido[3,4-b]indol-2-yl)-1,3-benzothiazole-6-carbonitrile Yellow solid was obtained with a yield of 41%. ¹H NMR (500 MHz, DMSO-d₆) δ 11.21 (s, 1H), 8.36 (s, 1H), 7.70 (d, J=8.4 Hz, 1H), 7.58 (d, J=8.4 Hz, 1H), 7.49 (d, J=2.1 Hz, 1H), 7.38 (d, J=8.6 Hz, 1H), 7.07 (d, J=8.6 Hz, 1H), 4.94 (s, 2H), 4.01 (d, J=6.3 Hz, 2H), 2.89 (d, J=5.3 Hz, 2H). LC-MS, Rt=6.330 min, [M+H]⁺=365.0.

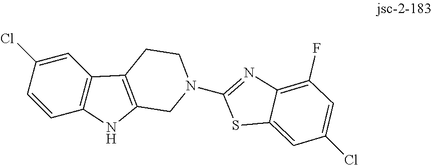 [SMILES: Fc1cc(Cl)cc2sc(N3CCc4c([nH]c5ccc(Cl)cc45)C3)nc12]

In another particular embodiment, the invention may include the compound: 6-chloro-2-(6-chloro-4-fluoro-1,3-benzothiazol-2-yl)-2,3,4,9-tetrahydro-1H-pyrido[3,4-b]indole Pale yellow solid was obtained with a yield of 74%. ¹H NMR (400 MHz, Chloroform-d) δ 7.94 (s, 1H), 7.44 (d, J=2.0 Hz, 1H), 7.37 (dd, J=2.0, 0.9 Hz, 1H), 7.24 (m, 1H), 7.12 (dd, J=8.6, 2.0 Hz, 1H), 7.06 (dd, J=10.4, 1.9 Hz, 1H), 4.95 (s, 2H), 3.93 (t, J=5.7 Hz, 2H), 2.95 (t, J=5.7 Hz, 2H). LC-MS, Rt=7.113 min, [M+H]⁺=392.0.

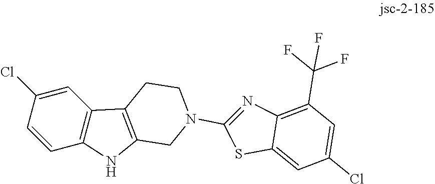 [SMILES: FC(F)(F)c1cc(Cl)cc2sc(N3CCc4c([nH]c5ccc(Cl)cc45)C3)nc12]

In another particular embodiment, the invention may include the compound: 6-chloro-2-[6-chloro-4-(trifluoromethyl)-1,3-benzothiazol-2-yl]-2,3,4,9-tetrahydro-1H-pyrido[3,4-b]indole Pale yellow solid was obtained with a yield of 82%. ¹H NMR (400 MHz, Chloroform-d) δ 7.96 (s, 1H), 7.62 (d, J=2.4 Hz, 1H), 7.57 (d, J=8.7 Hz, 1H), 7.51-7.47 (m, 1H), 7.41 (d, J=2.0 Hz, 1H), 7.23-7.18 (m, 1H), 7.11 (dd, J=8.6, 2.0 Hz, 1H), 5.15 (s, 2H), 4.12-4.04 (m, 2H), 2.86 (t, J=5.7 Hz, 2H). LC-MS, Rt=7.566 min, [M+H]⁺=442.0.

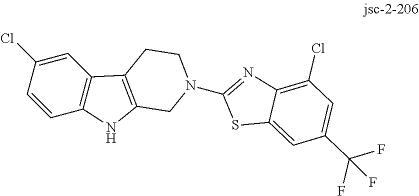 [SMILES: FC(F)(F)c1cc(Cl)c2nc(N3CCc4c([nH]c5ccc(Cl)cc45)C3)sc2c1]

In another particular embodiment, the invention may include the compound: 6-chloro-2-[4-chloro-6-(trifluoromethyl)-1,3-benzothiazol-2-yl]-2,3,4,9-tetrahydro-1H-pyrido[3,4-b]indole Pale yellow solid was obtained with a yield of 80%. ¹H NMR (500 MHz, Chloroform-d) δ 8.06 (s, 1H), 7.80-7.69 (m, 1H), 7.62-7.56 (m, 1H), 7.44 (d, J=2.0 Hz, 1H), 7.25 (d, J=8.6 Hz, 1H), 7.15 (dd, J=8.6, 2.0 Hz, 1H), 4.97 (s, 2H), 3.96 (t, J=5.7 Hz, 2H), 2.95 (t, J=5.7 Hz, 2H). LC-MS, Rt=7.409 min, [M+H]⁺=442.0.

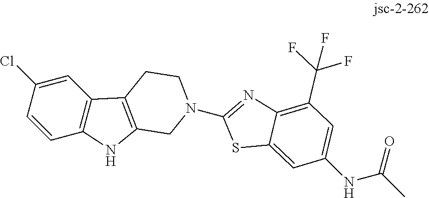 [SMILES: CC(=O)Nc1cc(C(F)(F)F)c2nc(N3CCc4c([nH]c5ccc(Cl)cc45)C3)sc2c1]

In another particular embodiment, the invention may include the compound: N-[2-(6-chloro-1,3,4,9-tetrahydro-2H-pyrido[3,4-b]indol-2-yl)-4-(trifluoromethyl)-1,3-benzothiazol-6-yl]acetamide To a round bottom flask was added 2-(6-chloro-1,3,4,9-tetrahydro-2H-pyrido[3,4-b]indol-2-yl)-4-(trifluoromethyl)-1,3-benzothiazol-6-aminium chloride (25 mg, 0.050 mmol), acetyl chloride (4.7 mg, 0.060 mmol), Et₃N (6.1 mg, 0.060 mmol), THF (2 mL), and the reaction mixture was stirred for 3 h. The solvents were removed under vacuum and the residue was purified by flash chromatography on silica gel using ethyl acetate/hexane (1:1, v/v) as eluent with a yield of 78%. LC-MS, Rt=9.281 min, [M+H]⁺=465.0.

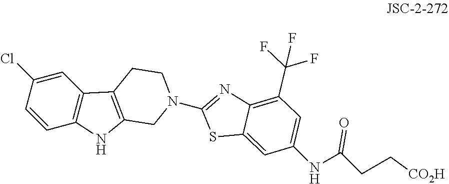 [SMILES: O=C(O)CCC(=O)Nc1cc(C(F)(F)F)c2nc(N3CCc4c([nH]c5ccc(Cl)cc45)C3)sc2c1]

In another particular embodiment, the invention may include the compound: 2-(6-chloro-1,3,4,9-tetrahydro-2H-pyrido[3,4-b]indol-2-yl)-4-oxo-4-{[4-(trifluoromethyl)-1,3-benzothiazol-6-yl]amino}butanoic acid To a round bottom flask was added 2-(6-chloro-1,3,4,9-tetrahydro-2H-pyrido[3,4-b]indol-2-yl)-4-(trifluoromethyl)-1,3-benzothiazol-6-aminium chloride (45 mg, 0.100 mmol), Succinic Anhydride (10 mg, 0.100 mmol) and CHCl₃ (2 mL), and the reaction mixture was refluxed for 4 h. The product was obtained as pale brown solid after filtration at room temperature with a yield of 57%. ¹H NMR (500 MHz, DMSO-d₆) δ 11.19 (s, 1H), 10.23 (s, 1H), 8.35 (d, J=2.0 Hz, 1H), 7.82 (d, J=2.1 Hz, 1H), 7.49 (d, J=2.1 Hz, 1H), 7.36 (d, J=8.5 Hz, 1H), 7.06 (dd, J=8.5, 2.2 Hz, 1H), 4.93 (s, 2H), 3.95 (t, J=5.8 Hz, 2H), 2.89 (t, J=5.9 Hz, 2H), 2.58-2.55 (d, J=5.5 Hz, 2H), 2.54 (d, J=5.5 Hz, 2H). LC-MS, Rt=9.165 min, [M+H]⁺=523.0.

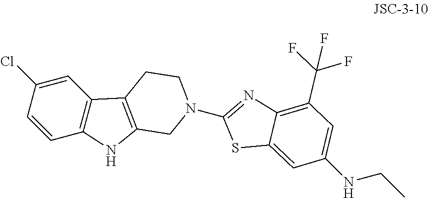 [SMILES: CCNc1cc(C(F)(F)F)c2nc(N3CCc4c([nH]c5ccc(Cl)cc45)C3)sc2c1]

In another particular embodiment, the invention may include the compound: 2-(6-chloro-1,3,4,9-tetrahydro-2H-pyrido[3,4-b]indol-2-yl)-N-ethyl-4-(trifluoromethyl)-1,3-benzothiazol-6-amine Prepared according the literature.³⁸ To a solution of 2-(6-chloro-1,3,4,9-tetrahydro-2H-pyrido[3,4-b]indol-2-yl)-4-(trifluoromethyl)-1,3-benzothiazol-6-aminium chloride (49 mg, 0.100 mmol, 1 equiv) in (CH₂Cl)₂ at room temperature were added acetaldehyde (5.5 mg, 0.120 mmol), AcOH (6.0 mg, 0.100 mmol) and NaHB(OAc)₃ (42.4 mg, 0.200 mmol) and the resulting mixture was stirred for 16 h. The solvent was removed in vacuo and the residue dissolved in ethyl acetate. The organic phase was washed with a saturated NaHCO₃aqueous solution, dried over Na₂SO₄ and concentrated in vacuo. The product was obtained after purification by flash chromatography on silica gel with a yield of 69%. ¹H NMR (400 MHz, Chloroform-d) δ 7.94 (s, 1H), 7.35 (d, J=2.0 Hz, 1H), 7.17-7.05 (m, 1H), 6.99 (d, J=2.7 Hz, 1H), 6.86 (d, J=2.3 Hz, 1H), 4.85 (s, 2H), 3.82 (t, J=5.7 Hz, 2H), 3.16 (q, J=7.1 Hz, 2H), 2.82 (t, J=5.7 Hz, 2H), 1.41-0.90 (m, 3H). LC-MS, Rt=8.356 min, [M+H]⁺=451.1.

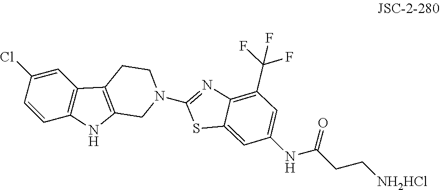

In another particular embodiment, the invention may include the compound: 2-(6-chloro-1,3,4,9-tetrahydro-2H-pyrido[3,4-b]indol-2-y)-3-oxo-3-{[4-(trifluoromethyl)-1,3-benzothiazol-6-yl]amino}propan-1-aminium chloride To a round bottom flask was added 2-(6-chloro-1,3,4,9-tetrahydro-2H-pyrido[3,4-b]indol-2-yl)-4-(trifluoromethyl)-1,3-benzothiazol-6-aminium chloride (25.0 mg, 0.050 mmol), Boc-P-Ala-OH (11.5 mg, 0.060 mmol), DMAP (7.5 mg, 0.060 mmol), EDCI (9.5 mg, 0.060 mmol) and DCM (2 mL), and the reaction mixture was stirred for 2 h. The solvents were removed under vacuum and the residue was purified by flash chromatography on silica gel using ethyl acetate/hexane as eluent. To a round bottom flask was added the obtained product (25 mg, 0.05 mmol) and HCl in dioxane (2.0 mL, 4 M), and the reaction mixture was stirred for 4 h. The solvents were removed under vacuum and red solid was obtained as product with a yield of 90%. ¹H NMR (500 MHz, Methanol-d₄) δ 8.27 (d, J=2.1 Hz, 1H), 7.76 (d, J=2.1 Hz, 1H), 7.40 (d, J=2.0 Hz, 1H), 7.28 (d, J=8.6 Hz, 1H), 7.04 (dd, J=8.5, 2.1 Hz, 1H), 4.94 (s, 2H), 4.01 (t, J=5.8 Hz, 2H), 3.34-3.25 (m, 2H), 2.92 (t, J=5.8 Hz, 2H), 2.85 (t, J=6.2 Hz, 2H). LC-MS, Rt=8.753 min, [M−Cl]⁺=494.1.

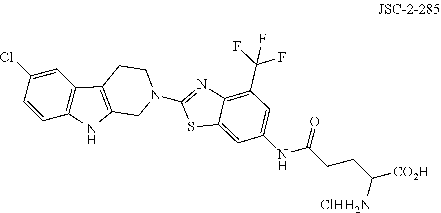

In another particular embodiment, the invention may include the compound: 2-(6-chloro-1,3,4,9-tetrahydro-2H-pyrido[3,4-b]indol-2-yl)-5-amino-1,5-dioxo-1-{[4-(trifluoromethyl)-1,3-benzothiazol-6-yl]amino}pentan-2-aminium chloride (4ax) To a round bottom flask was added 2-(6-chloro-1,3,4,9-tetrahydro-2H-pyrido[3,4-b]indol-2-yl)-4-(trifluoromethyl)-1,3-benzothiazol-6-aminium chloride (25.0 mg, 0.050 mmol), Boc-Gln-OH (14.8 mg, 0.060 mmol), DMAP (7.5 mg, 0.060 mmol), EDCI (9.5 mg, 0.060 mmol) and DCM (2 mL), and the reaction mixture was stirred for 2 h. The solvents were removed under vacuum and the residue was purified by flash chromatography on silica gel using ethyl acetate/hexane as eluent. To a round bottom flask was added the obtained product (30 mg, 0.050 mmol) and HCl in dioxane (2.0 mL, 4 M), and the reaction mixture was stirred for 4 h. The solvents were removed under vacuum and red solid was obtained as product with a yield of 90%. ¹H NMR (500 MHz, Methanol-d₄) δ 8.35-8.24 (m, 1H), 7.85-7.70 (m, 1H), 7.42-7.35 (m, 1H), 7.26 (dd, J=8.5, 3.5 Hz, 1H), 7.03 (dt, J=8.5, 2.2 Hz, 1H), 4.20-4.11 (m, 1H), 4.02-3.92 (m, 2H), 3.45 (t, J=7.1 Hz, 1H), 3.32 (dq, J=3.3, 1.9 Hz, 3H), 2.90 (t, J=5.8 Hz, 2H), 2.84 (s, 1H), 2.55 (t, J=6.9 Hz, 2H), 2.45-2.36 (m, 1H), 2.26 (dp, J=21.5, 7.3 Hz, 2H). LC-MS, Rt=7.615 min, [M−Cl]⁺=551.1.

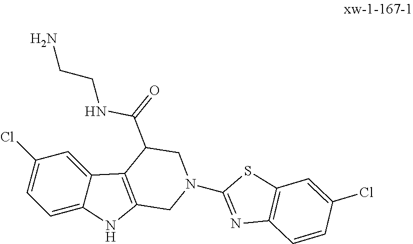 [SMILES: NCCNC(=O)C1CN(c2nc3ccc(Cl)cc3s2)Cc2[nH]c3ccc(Cl)cc3c21]

In another particular embodiment, the invention may include the compound: N-(2-aminoethyl)-6-chloro-2-(6-chlorobenzo[d]thiazol-2-yl)-2,3,4,9-tetrahydro-1H-pyrido[3,4-b]indole-4-carboxamide. Prepared by refluxing the mixture of the ester (20 mg, 0.046 mmol) and ethane-1,2-diamine (6.68 mmol, 0.1 ml) in 1 ml MeOH to afford the title compound (12 mg, 57%). ¹H NMR (500 MHz, DMSO-d₆) δ 11.32 (s, 1H), 8.15 (t, J=5.8 Hz, 1H), 7.94 (d, J=2.3 Hz, 1H), 7.46 (d, J=8.6 Hz, 1H), 7.41 (d, J=2.1 Hz, 1H), 7.39 (d, J=8.6 Hz, 1H), 7.31 (dd, J=8.6, 2.3 Hz, 1H), 7.07 (dd, J=8.6, 2.1 Hz, 1H), 4.89 (q, J=16.2 Hz, 2H), 4.17-4.05 (m, 1H), 4.01 (dd, J=13.2, 6.4 Hz, 1H), 3.95 (d, J=5.8 Hz, 1H), 3.17 (d, J=4.6 Hz, 2H), 3.16-3.10 (m, 1H), 3.05 (dd, J=12.7, 6.3 Hz, 1H), 2.59 (h, J=6.1 Hz, 2H). LC-MS, Rt=5.406 min, [M+H]⁺=462.1.

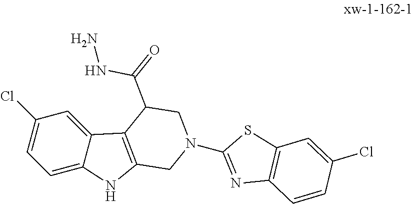 [SMILES: NNC(=O)C1CN(c2nc3ccc(Cl)cc3s2)Cc2[nH]c3ccc(Cl)cc3c21]

In another particular embodiment, the invention may include the compound: 6-chloro-2-(6-chlorobenzo[d]thiazol-2-yl)-2,3,4,9-tetrahydro-1H-pyrido[3,4-b]indole-4-carbohydrazide. Prepared by refluxing the mixture of the ester (10 mg, 0.023 mmol) and hydrazine (3.14 mmol, 0.1 ml) in 0.5 ml ethanol to afford the title compound (9 mg, 90%). LC-MS, Rt=6.393 min, [M+H]⁺=432.0.

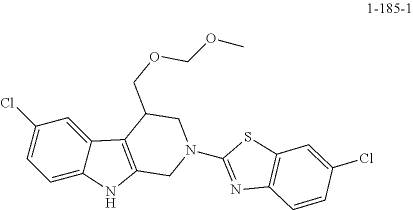 [SMILES: COCOCC1CN(c2nc3ccc(Cl)cc3s2)Cc2[nH]c3ccc(Cl)cc3c21]

In another particular embodiment, the invention may include the compound: 6-chloro-2-(6-chloro-4-((methoxymethoxy)methyl)-1,3,4,9-tetrahydro-2H-pyrido[3,4-b]indol-2-yl)benzo[d]thiazole. To the mixture of alcohol (15 mg, 0.0371 mmol) and N,N-Diisopropylethylamine (20 ul, 0.112 mmol) in anhydrous DCM was added MOMCI (10 ul, 0.132 mmol). The reaction mixture was stirred at room temperature for 2h. LCMS checked and the alcohol was gone. Removed all the solvent and purified by flash chromatography to afford the desired product (5.0 mg, 30%). ¹H NMR (500 MHz, Chloroform-d) δ 8.13 (s, 1H), 7.59 (d, J=2.1 Hz, 1H), 7.54 (d, J=2.0 Hz, 1H), 7.45 (d, J=8.6 Hz, 1H), 7.27-7.22 (m, 2H), 7.14 (dd, J=8.6, 2.0 Hz, 1H), 5.24 (d, J=16.3 Hz, 1H), 4.77-4.67 (m, 2H), 4.65 (dd, J=16.3, 1.5 Hz, 1H), 4.23 (dd, J=13.2, 2.6 Hz, 1H), 3.86 (dd, J=9.9, 4.3 Hz, 1H), 3.73 (dd, J=13.2, 3.9 Hz, 1H), 3.55 (t, J=9.9 Hz, 1H), 3.41 (s, 3H). LC-MS, Rt=8.249 min, [M+H]⁺=448.0.

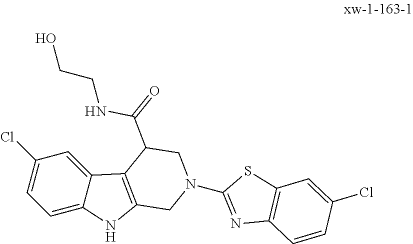 [SMILES: O=C(NCCO)C1CN(c2nc3ccc(Cl)cc3s2)Cc2[nH]c3ccc(Cl)cc3c21]

In another particular embodiment, the invention may include the compound: 6-chloro-2-(6-chlorobenzo[d]thiazol-2-yl)-N-(2-hydroxyethyl)-2,3,4,9-tetrahydro-1H-pyrido[3,4-b]indole-4-carboxamide Prepared by refluxing the mixture of the ester (20 mg, 0.046 mmol) and ethanolamine (0.46 mmol, 28 mg) to afford the title compound (9 mg, 42%). ¹H NMR (500 MHz, DMSO-d₆) δ 11.32 (s, 1H), 8.21 (t, J=5.7 Hz, 1H), 7.95 (d, J=2.3 Hz, 1H), 7.47 (d, J 8.6 Hz, 1H), 7.42-7.39 (m, 2H), 7.31 (dd, J=8.6, 2.2 Hz, 1H), 7.07 (dd, J=8.5, 2.1 Hz, 1H), 4.97-4.82 (m, 2H), 4.10 (q, J=7.7 Hz, 1H), 3.98 (q, J=5.5, 4.4 Hz, 2H), 3.45-3.39 (m, 2H), 3.18 (ddt,J=30.2, 13.1, 6.4 Hz, 2H). LC-MS, Rt=7.970 min, [M+H]⁺=461.0.

TABLE 1
Initial screening of tryptoline-based structure for the RMA activity.

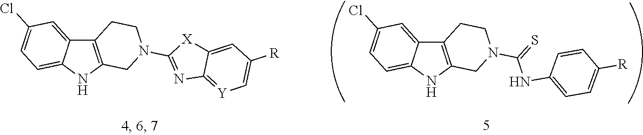

| Compd | X | Y | R | MIC^{a} | MRC^{b} |
|---|---|---|---|---|---|
| 4a | S | C | Cl | >64 | 2 |
| 4b | S | C | H | >64 | >64 |
| 4c | S | C | Me | >64 | >64 |
| 4d | S | N | H | >64 | >64 |
| 4e | S | N | CF₃ | >64 | >64 |
| 5a | S | C | Cl | 4 | 2 |
| 5b | S | C | OCF₃ | 2 | 1 |
| 5c | S | C | CF₃ | 2 | 1 |
| 6a | NMe | C | H | >64 | >64 |
| 6b | NMe | C | Cl | >64 | >64 |
| 6c | NMe | C | CF₃ | >64 | >64 |
| 7a | O | C | H | >64 | >64 |
| 7b | O | C | Cl | >64 | >64 |
| 7c | O | C | CF₃ | >64 | >64 |

All MIC values are determined by using MRSA BAA-44 strain and reported in μg/mL.
All MRC values are determined by using MRSA BAA-44 in combination with cefazolin and reported in μg/mL.

TABLE 2
Optimization of tryptoline motif for the RMA activity.

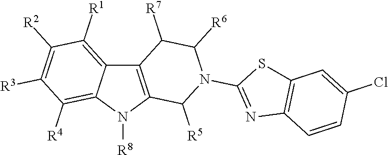

| Compd | R¹/R²/R³/R⁴ | R⁵ | R⁶ | R⁷ | R⁸ | MIC^{a} | MRC^{b} |
|---|---|---|---|---|---|---|---|
| 4a | H/Cl/H/H | H | H | H | H | >64 | 2 |
| 4f | Cl/H/H/H | H | H | H | H | >16 | 16 |
| 4g | H/H/Cl/H | H | H | H | H | >64 | >64 |
| 4h | H/H/H/Cl | H | H | H | H | >16 | 16 |
| 4i | H/F/H/H | H | H | H | H | >64 | >64 |
| 4j | H/Br/H/H | H | H | H | H | 4 | 1 |
| 4k | H/OMe/H/H | H | H | H | H | >64 | >64 |
| 4l | H/Cl/H/H | CO₂Et | H | H | H | >64 | >64 |
| 4m | H/Cl/H/H | H | CO₂Me | H | H | >64 | >64 |
| 4n | H/Cl/H/H | H | H | CO₂Me | H | >32 | 1 |
| 4o | H/Cl/H/H | H | H | H | Et | >64 | >64 |

All MIC values are determined by using MRSA BAA-44 strain and reported in μg/mL.
All MRC values are determined by using MRSA BAA-44 in combination with cefazolin and reported in μg/mL.

TABLE 3
Optimization of benzothiazole motif for the RMA activity.

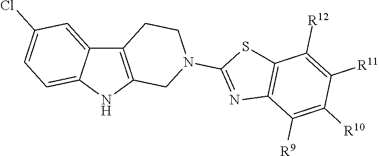

| Compd | R⁹ | R¹⁰ | R¹¹ | R¹² | MIC^{a} | MRC^{b} | GI₅₀ |
|---|---|---|---|---|---|---|---|
| 4a | H | H | Cl | H | >64 | 2 | 13.2 |
| 4p | H | H | Br | H | >64 | 2 | 5.1 |
| 4q | H | H | NH, | H | >64 | >64 | NT^{d} |
| 4r | H | H | CO₂Et | H | >64 | >64 | NT^{d} |
| 4s | H | H | CF₃ | H | >64 | 0.5 | 4.6 |
| 4t | H | H | OCF₃ | H | 1 | 1 | 7.3 |
| 4u | H | H | H | Cl | 4 | 1 | 6.7 |
| 4v | H | Cl | H | H | >64 | >64 | NT^{d} |
| 4w | Cl | H | H | H | >64 | 2 | 27.7 |
| 4x | Cl | H | Cl | H | 2 | 1 | 16.1 |
| 4y | Cl | Cl | Cl | H | 2 | 1 | 11.7 |
| 4z | Cl | H | OMe | H | >32 | >32 | NT^{d} |
| 4aa | Cl | H | OH | H | >32 | 8 | 5.5 |
| 4ab | Cl | H | OCH₂CH₂NH₂ | H | 8 | 4 | NT^{d} |
| 4ac | Cl | H | Br | H | >32 | 8 | 22 |
| 4ad | Cl | H | NH | H | >32 | 2 | >100 |
| 4ae | Cl | H | NHCOCH₂NH₃Cl | H | 2 | 2 | 14.8 |
| 4af | CF₃ | H | H | H | >32 | 1 | 8 |
| 4ag | CF₃ | H | Br | H | 4 | 0.5 | 5.9 |
| 4ah | CF₃ | H | NH₂HCl | H | 16 | 2 | 32 |
| 4ai | CF₃ | H | NHCH₃ | H | >32 | 2 | 15.4 |
| 4aj | CF₃ | H | NH(CH₂)₂NH₃Cl | H | 1 | 1 | 7.4 |

All MIC values are determined by using MRSA BAA-44 strain and reported in μg/mL.
All MRC values are determined by using MRSA BAA-44 in combination with cefazolin and reported in μg/mL; ^{c}HeLa was used for determination of GI₅₀ and reported in μg/mL; ^{d}GI₅₀ was not test.

TABLE 4
Further optimization of tryptoline motif on R⁷ for the RMA activity.

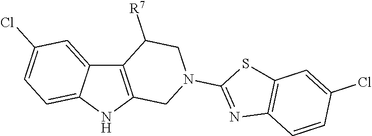

| Compd | R⁷ | MIC^{a} | MRC^{b} | GI₅₀^{c} |
|---|---|---|---|---|
| 4n | CO₂Me | >32 | 1 | 6.6 |
| 8 | COOH | 32 | 16 | 65 |
| 9 | CONMe₂ | >32 | >32 | NT^{d} |
| 10 | CH₂OH | 2 | 2 | 10.8 |
| 11 | CH₂OCH₂OMe | 2 | 2 | 7.0 |
| 12a | CH₂SCH₂CH₂NH₃Cl | 1 | 1 | 3.8 |
| 12b | CH₂NH₂ | 1 | 1 | 1.5 |
| 12c | CH₂NMe₂ | 2 | 1 | 5.2 |
| 12d | CH₂N⁺Me₃I⁻ | 8 | 4 | 18 |
| 12e | CH₂NHCH₂CH₂NH₂ | 2 | 2 | 2.4 |
| 12f | CH₂NHCH═NH | 4 | 2 | 3.8 |
| 12g | CH₂NHC═NHNH₃Cl | 2 | 1 | 14.1 |

All MIC values are determined by using MRSA BAA-44 strain and reported in μg/mL.
All MRC values are determined by using MRSA BAA-44 in combination with cefazolin and reported in μg/mL; ^{c}HeLa was used for determination of GI₅₀ and reported in μg/mL; ^{d}GI₅₀ was not test.

TABLE 5
Evaluation of the MRC values of 4ad in a panel of MRSA.

| Strains | MIC^{a} | MRC (cefazolin)^{b} | MRC (cefuroxime)^{c} |
|---|---|---|---|
| MRSA BAA-1683 | >32 | 1 | 8 |
| MRSA BAA-1764 | >32 | 0.5 | 32 |
| MRSA NR-46411 | >32 | 0.5 | <0.25 |
| NRS-384 | >32 | 4 | 4 |
| NRS-100 | >32 | 4 | 4 |

All MIC values are determined by using corresponding strain and reported in μg/mL;
All MRC values are determined by using corresponding strain in combination with cefazolin and reported in μg/mL; ^{c}All MRC values are determined by using corresponding strain in combination with cefuroxime and reported in μg/mL.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### REFERENCES

The following references are incorporated in their entirety herein by reference:
1. Davies, J., Microbiologia 1996, 12, 9-16.
2. Lowy, F. D., J Clin Invest 2003, 111, 1265-1273.
3. Bush, K., Clin Microbiol Infect 2004, 10 Suppl 4, 10-17.
4. Stryjewski, M. E.; Corey, G. R., Clin Infect Dis 2014, 58 Suppl 1, S10-9.
5. D'Costa, V. M.; King, C. E.; Kalan, L.; Morar, M.; Sung, W. W.; Schwarz, C.; Froese, D.; Zazula, G.; Calmels, F.; Debruyne, R.; Golding, G. B.; Poinar, H. N.; Wright, G. D., Nature 2011, 477, 457-461.
6. Kim, W.; Hendricks, G. L.; Tori, K.; Fuchs, B. B.; Mylonakis, E., Future Med Chem 2018, 10, 779-794.
7. Brown, E. D.; Wright, G. D., Nature 2016, 529, 336-343.
8. Ali, J.; Rafiq, Q. A.; Ratcliffe, E., Future Sci OA 2018, 4, FSO290.
9. Staph infection can kill, Cent. Dis. Control Prev. 2019.
   cdc.gov/vitalsigns/staph/pdf/vs-0305-staph-infections-H.pdf (accessed May 25, 2019).
10. Rice, L. B., J Infect Dis 2008, 197, 1079-1081.
11. Richter, M. F.; Hergenrother, P. J., Ann NY Acad Sci 2019, 1435, 18-38.
12. Richter, M. F.; Drown, B. S.; Riley, A. P.; Garcia, A.; Shirai, T.; Svec, R. L.; Hergenrother, P. J., Nature 2017, 545, 299-304.
13. Abreu, A. C.; McBain, A. J.; Simoes, M., Nat Prod Rep 2012, 29, 1007-1021.
14. Pasquina, L.; Santa Maria, J. P., Jr.; McKay Wood, B.; Moussa, S. H.; Matano, L.

M.; Santiago, M.; Martin, S. E.; Lee, W.; Meredith, T. C.; Walker, S., Nat Chem Biol 2016, 12, 40-45.
15. Kalan, L.; Wright, G. D., Expert Rev Mol Med 2011, 13, e5.
16. Podoll, J. D.; Liu, Y.; Chang, L.; Walls, S.; Wang, W.; Wang, X., Proc Natl Acad Sci USA 2013, 110, 15573-15578.
17. Chang, L.; Podoll, J. D.; Wang, W.; Walls, S.; O'Rourke, C. P.; Wang, X., J Med Chem 2014, 57, 3803-3817.
18. Michael Barbour, P.; Podoll, J. D.; Marholz, L. J.; Wang, X., Boorg Med Chem Lett 2014, 24, 5602-5605.
19. Xu, W.; Wang, W.; Wang, X., Angew Chem Int Ed Engl2015, 54, 9546-9549.
20. Barbour, P. M.; Wang, W.; Chang, L.; Pickard, K. L.; Rais, R.; Slusher, B. S.; Wang, X., Adv Synth Catal 2016, 358, 1482-1490.
21. Zhu, Y.; He, W.; Wang, W.; Pitsch, C. E.; Wang, X.; Wang, X., Angew Chem Int Ed Engi2017, 56, 12206-12209.
22. Bansal, Y.; Silakari, O., Bioorg Med Chem 2012, 20, 6208-36.
23. Keri, R. S.; Hiremathad, A.; Budagumpi, S.; Nagaraja, B. M., Chem Biol Drug Des 2015, 86, 799-845.
24. Demmer, C. S.; Bunch, L., Eur. J. Med. Chem. 2015, 97, 778-785.
25. Keri, R. S.; Patil, M. R.; Patil, S. A.; Budagupi, S., Eur. J. Med. Chem. 2015, 89, 207-251.
26. Gjorgjieva, M.; Tomasic, T.; Barancokova, M.; Katsamakas, S.; Ilas, J.; Tammela, P.; Peterlin Masic, L.; Kikelj, D., J. Med. Chem. 2016, 59, 8941-8954.
27. Alborz, M.; Jarrahpour, A.; Pournejati, R.; Karbalaei-Heidari, H. R.; Sinou, V.; Latour, C.; Brunel, J. M.; Sharghi, H.; Aberi, M.; Turos, E.; Wojtas, L., Eur. J. Med. Chem. 2018, 143, 283-291.
28. Haroun, M.; Tratrat, C.; Kositzi, K.; Tsolaki, E.; Petrou, A.; Aldhubiab, B.; Attimarad, M.; Harsha, S.; Geronikaki, A.; Venugopala, K. N.; Elsewedy, H. S.; Sokovic, M.; Glamoclija, J.; Ciric, A., Curr. Top. Med. Chem. (Sharjah, United Arab Emirates) 2018, 18, 75-87.
29. Naaz, F.; Srivastava, R.; Singh, A.; Singh, N.; Verma, R.; Singh, V. K.; Singh, R. K., Bioorg. Med. Chem. 2018, Ahead of Print.
30. L., M. S.; E., H. C.; Qiu, W., Angew. Chem., Int. Ed. 2014, 53, 4667-4670.
31. Megyesi, R.; Mandi, A.; Kurtan, T.; Forro, E.; Fulop, F., Eur J Org Chem 2017, 4713-4718.
32. Bartoli, G.; Bosco, M.; Giuli, S.; Giuliani, A.; Lucarelli, L.; Marcantoni, E.; Sambri, L.; Torregiani, E., J Org Chem 2005, 70, 1941-1944.
33. Monecke, S.; Coombs, G.; Shore, A. C.; Coleman, D. C.; Akpaka, P.; Borg, M.; Chow, H.; Ip, M.; Jatzwauk, L.; Jonas, D.; Kadlec, K.; Kearns, A.; Laurent, F.; O'Brien, F. G.; Pearson, J.; Ruppelt, A.; Schwarz, S.; Scicluna, E.; Slickers, P.; Tan, H. L.; Weber, S.; Ehricht, R., PLoS One 2011, 6, e17936.
34. Centers for Disease, C.; Prevention, MMWR Morb Mortal Wkly Rep 2002, 51, 902.
35. Centers for Disease, C.; Prevention, MMWR Morb Mortal Wkly Rep 2003, 52, 793-795.
36. Cassat, J. E.; Dunman, P. M.; McAleese, F.; Murphy, E.; Projan, S. J.; Smeltzer, M. S., J Bacteriol 2005, 187, 576-592.
37. Holden, M. T.; Feil, E. J.; Lindsay, J. A.; Peacock, S. J.; Day, N. P.; Enright, M. C.; Foster, T. J.; Moore, C. E.; Hurst, L.; Atkin, R.; Barron, A.; Bason, N.; Bentley, S. D.; Chillingworth, C.; Chillingworth, T.; Churcher, C.; Clark, L.; Corton, C.; Cronin, A.; Doggett, J.; Dowd, L.; Feltwell, T.; Hance, Z.; Harris, B.; Hauser, H.; Holroyd, S.; Jagels, K.; James, K. D.; Lennard, N.; Line, A.; Mayes, R.; Moule, S.; Mungall, K.; Ormond, D.; Quail, M. A.; Rabbinowitsch, E.; Rutherford, K.; Sanders, M.; Sharp, S.; Simmonds, M.; Stevens, K.; Whitehead, S.; Barrell, B. G.; Spratt, B. G.; Parkhill, J., Proc Natl Acad Sci USA 2004, 101, 9786-9791.
38. Kunst, F.; Ogasawara, N.; Moszer, I.; Albertini, A. M.; Alloni, G.; Azevedo, V.; Bertero, M. G.; Bessieres, P.; Bolotin, A.; Borchert, S.; Borriss, R.; Boursier, L.; Brans, A.; Braun, M.; Brignell, S. C.; Bron, S.; Brouillet, S.; Bruschi, C. V.; Caldwell, B.; Capuano, V.; Carter, N. M.; Choi, S. K.; Cordani, J. J.; Connerton, I. F.; Cummings, N. J.; Daniel, R. A.; Denziot, F.; Devine, K. M.; Dusterhoft, A.; Ehrlich, S. D.; Emmerson, P. T.; Entian, K. D.; Errington, J.; Fabret, C.; Ferrari, E.; Foulger, D.; Fritz, C.; Fujita, M.; Fujita, Y.; Fuma, S.; Galizzi, A.; Galleron, N.; Ghim, S. Y.; Glaser, P.; Goffeau, A.; Golightly, E. J.; Grandi, G.; Guiseppi, G.; Guy, B. J.; Haga, K.; Haiech, J.; Harwood, C. R.; Henaut, A.; Hilbert, H.; Holsappel, S.; Hosono, S.; Hullo, M. F.; Itaya, M.; Jones, L.; Joris, B.; Karamata, D.; Kasahara, Y.; Klaerr-Blanchard, M.; Klein, C.; Kobayashi, Y.; Koetter, P.; Koningstein, G.; Krogh, S.; Kumano, M.; Kurita, K.; Lapidus, A.; Lardinois, S.; Lauber, J.; Lazarevic, V.; Lee, S. M.; Levine, A.; Liu, H.; Masuda, S.; Mauel, C.; Medigue, C.; Medina, N.; Mellado, R. P.; Mizuno, M.; Moestl, D.; Nakai, S.; Noback, M.; Noone, D.; O'Reilly, M.; Ogawa, K.; Ogiwara, A.; Oudega, B.; Park, S. H.; Parro, V.; Pohl, T. M.; Portelle, D.; Porwollik, S.; Prescott, A. M.; Presecan, E.; Pujic, P.; Purnelle, B.; Rapoport, G.; Rey, M.; Reynolds, S.; Rieger, M.; Rivolta, C.; Rocha, E.; Roche, B.; Rose, M.; Sadaie, Y.; Sato, T.; Scanlan, E.; Schleich, S.; Schroeter, R.; Scoffone, F.; Sekiguchi, J.; Sekowska, A.; Seror, S. J.; Serror, P.; Shin, B. S.; Soldo, B.; Sorokin, A.; Tacconi, E.; Takagi, T.; Takahashi, H.; Takemaru, K.; Takeuchi, M.; Tamakoshi, A.; Tanaka, T.; Terpstra, P.; Togoni, A.; Tosato, V.; Uchiyama, S.; Vandebol, M.; Vannier, F.; Vassarotti, A.; Viari, A.; Wambutt, R.; Wedler, H.; Weitzenegger, T.; Winters, P.; Wipat, A.; Yamamoto, H.; Yamane, K.; Yasumoto, K.; Yata, K.; Yoshida, K.; Yoshikawa, H. F.; Zumstein, E.; Yoshikawa, H.; Danchin, A., Nature 1997, 390, 249-256.
39. Nallapareddy, S. R.; Singh, K. V.; Murray, B. E., Appl Environ Microbiol 2006, 72, 334-345.
40. Van Schaik, W.; Top, J.; Riley, D. R.; Boekhorst, J.; Vrijenhoek, J. E.; Schapendonk, C. M.; Hendrickx, A. P.; Nijman, I. J.; Bonten, M. J.; Tettelin, H.; Willems, R. J., BMC Genomics 2010, 11, 239.
41. Knez, D.; Brus, B.; Coquelle, N.; Sosi6, I.; § ink, R.; Brazzolotto, X.; Mravljak, J.; Colletier, J.-P.; Gobec, S., Bioorg. Med Chem. 2015, 23, 4442-4452.
42. Liu, X.; Wang, X.; Li, Q.; Kozar, M. P.; Melendez, V.; O'Neil, M. T.; Lin, A. J., J. Med Chem. 2011, 54, 4523-4535.
43. Joyce, L. L.; Batey, R. A., Org. Lett. 2009, 11, 2792-2795.
44. Megyesi, R.; Mandi, A.; Kurtan, T.; Forro, E.; Fueloep, F., Eur. J. Org. Chem. 2017, 2017, 4713-4718.
45. Bhunia, S. S.; Misra, A.; Khan, I. A.; Gaur, S.; Jain, M.; Singh, S.; Saxena, A.; Hohlfield, T.; Dikshit, M.; Saxena, A. K., J. Med Chem. 2017, 60, 322-337.
46. Jorgensen, W. L.; Bollini, M.; Thakur, V. V.; Domaoal, R. A.; Spasov, K. A.; Anderson, K. S., J. Am. Chem. Soc. 2011, 133, 15686-15696.
47. Valhondo, M.; Marco, I.; Martin-Fontecha, M.; Vizquez-Villa, H.; Ramos, J. A.; Berkels, R.; Lauterbach, T.; Benhamú, B.; López-Rodriguez, M. L., J. Med Chem. 2013, 56, 7851-7861.
48. Xu, H.; Wolf, C., Chem Commun (Camb) 2009, 3035-3037.
49. Sengoden, M.; Bhowmick, A.; Punniyamurthy, T., Org Lett 2017, 19, 158-161.
50. Charrier, N.; Clarke, B.; Cutler, L.; Demont, E.; Dingwall, C.; Dunsdon, R.; East, P.; Hawkins, J.; Howes, C.; Hussain, I.; Jeffrey, P.; Maile, G.; Matico, R.; Mosley, J.; Naylor, A.; O'Brien, A.; Redshaw, S.; Rowland, P.; Soleil, V.; Smith, K. J.; Sweitzer, S.; Theobald, P.; Vesey, D.; Walter, D. S.; Wayne, G., J Med Chem 2008, 51, 3313-3317.

1-26. (canceled)

27. A compound of the formula (I): 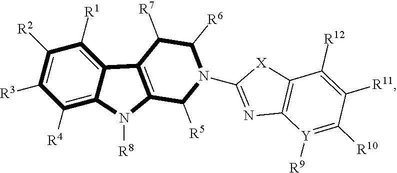
or a stereoisomer, pharmaceutically acceptable salt thereof, wherein,
X is independently S, O, N, or NMe;
Y is independently C, or N;
R¹ is independently H, or Cl;
R² is independently Cl, H, F, Br, OH, CH₃, or OMe;
R³ is independently H, or Cl;
R⁴ is independently H, or Cl;
R⁵ is independently H, or CO₂Et;
R⁶ is independently H, or CO₂Me;
R⁷ is independently a unsubstituted aromatic, a substitute aromatic, an alkyle halide, an amide, an amine, an alkane, an alkene, an alkyne, a nitrile, H, CO₂Me, CH₃, COO⁻, CONH₂, CON(CH₃)₂, COOH, CONMe₂, CH₂OH, CH₂OCH₂OMe, CH₂SCH₂CH₂NH₃Cl, CH₂NH₂, CH₂NMe₂, CH₂N⁺Me₃I⁻, CH₂NHCH₂CH₂NH₂, CH₂NHCH═NH, (CH₂)₅NH, or CH₂NHC═NHNH₃Cl, or R⁷ is independently: 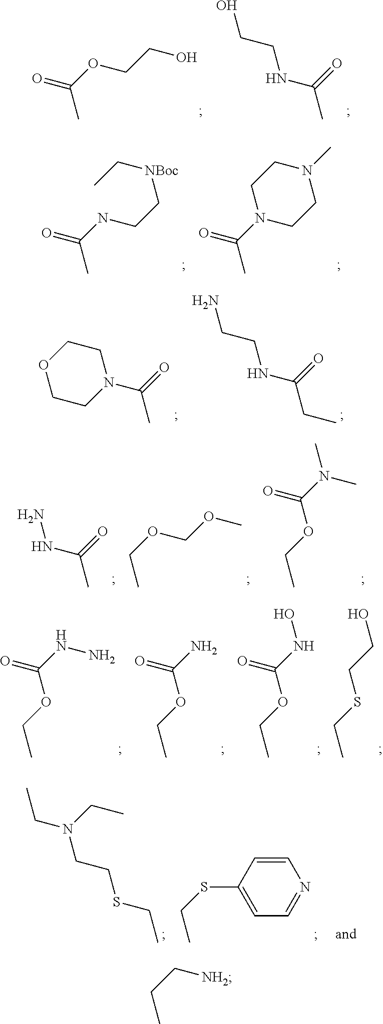
R⁸ is independently H, or Et;
R⁹ is independently H, Cl, or CF₃;
R¹⁰ is independently an alkyle halide, an amide, an amine, an alkane, an alkene, an alkyne, a nitrile, Cl, F, H, Me, Br, CF₃, NO₂, CN, OCF₃, NH₂, OMe, OH, SO₂CH₃, SO₂CH₃HCL, 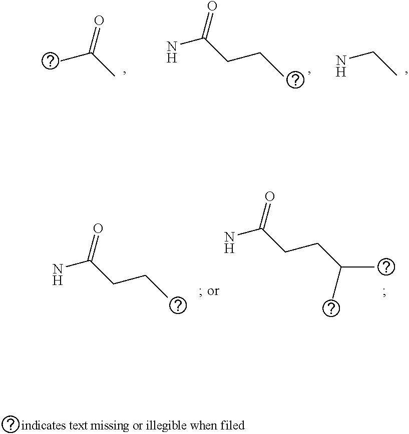
R¹¹ is independently an alkyle halide, an amine, an alkane, an alkene, an alkyne, a nitrile, H, Cl, Br, NH₂, OH, Me, CN, OMe, OCH₂CH₂NH₂, NHCOCH₂NH₃Cl, NH₂HCl, NHCH₃, NH(CH₂)₂NH₃HCl, CO₂HCH₂CH₃, or CF₃; and
R¹² is independently H, alkyle halide, F, CF₃, or Cl.

28. A compound of the formula (I): 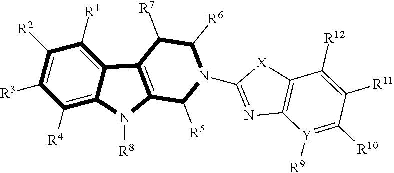
or a stereoisomer, pharmaceutically acceptable salt thereof, wherein,
X is independently S;
Y is independently C;
R¹ is independently H;
R² is independently Cl, or Br;
R³ is independently H;
R⁴ is independently H;
R⁵ is independently H;
R⁶ is independently H;
R⁷ is independently H, CO₂Me, COOH, CONMe₂, CH₂OH, CH₂OCH₂OMe, CH₂SCH₂CH₂NH₃Cl, CH₂NH₂, CH₂NMe₂, CH₂N⁺Me₃I⁻, CH₂NHCH₂CH₂NH₂, CH₂NHCH═NH, (CH₂)₅NH, or CH₂NHC═NHNH₃Cl;
R⁸ is independently H;
R⁹ is independently H, and wherein R11 is selected from the group consisting of: Cl, Br, CF₃, OCF₃; or
R⁹ is independently Cl, and wherein R11 is selected from the group consisting of: H, Cl, Br, NH₂, or
R⁹ is independently CF₃, and wherein R11 is selected from the group consisting of: H, NH₂HCl, NHCH₃,
R¹⁰ is independently H, or CF₃; and
R¹² is independently H, or Cl.

29-41. (canceled)
